(19) United States
(12) Patent Application Publication
(10) Pub. No.: US 2023/0065900 A1
(43) Pub. Date: Mar. 2, 2023

(54) COMPOSITIONS AND METHODS FOR MODULATING INFLAMMATION AND WOUND HEALING

(71) Applicant: Alastin Skincare, Inc., Carlsbad, CA (US)

(72) Inventors: Alan David WIDGEROW, Carlsbad, CA (US); John A. GARRUTO, Carlsbad, CA (US)

(73) Assignee: Alastin Skincare, Inc., Carlsbad, CA (US)

(21) Appl. No.: 17/981,862

(22) Filed: Nov. 7, 2022

Related U.S. Application Data
(63) Continuation of application No. PCT/US2021/031244, filed on May 7, 2021.
(60) Provisional application No. 63/022,209, filed on May 8, 2020.

(51) Int. Cl. C07K 5/08; C07K 7/06; A61K 9/00; A61K 31/685; A61K 31/351; A61P 17/02

(52) U.S. Cl. CPC C07K 5/08; C07K 7/06; A61K 9/0014; A61K 31/685; A61K 31/351; A61P 17/02

(57) ABSTRACT

Compositions and methods for modulating inflammation and wound healing are provided.

### CROSS-REFERENCE

This application is a continuation of International Application No. PCT/US2021/031244 filed May 7, 2021, which claims the benefit of U.S. Provisional Patent Application No. 63/022,209 filed May 8, 2020, which is incorporated by reference in its entirety.

### BACKGROUND

The industry for skin procedures or surgical procedures such as cosmetic surgeries, facial aesthetics, and medical lasers is estimated to be in the billions. Due to this significant industry growth, the need for skincare treatments effective at promoting skin regeneration and wound healing as well as alleviating the negative side effects of invasive skin treatments is also rapidly growing. These negative side effects are often a result of slow and ineffective skin regeneration or wound healing, which produces prolonged periods of inflammation, skin sensitivity, scarring, bruising, dry skin, infection, and other unfortunate skin conditions. Thus, there is a need for compositions that can accelerate the wound healing process, reduce pain or discomfort, and accelerate resolution of inflammation to be used with skin procedures or surgical procedures.

### SUMMARY

Compositions and methods are described herein for modulating inflammatory or regenerative gene expression. In some instances, the compositions and methods as described herein accelerate the healing process, accelerate clearance of products including lipid particles, induce inflammatory and regenerative gene expression, accelerate resolution of inflammation stimulates extracellular matrix remodeling, reduce induration, reduce fibrous banding, reduce pain or discomfort, or combinations thereof.

An aspect described herein are methods for improving wound healing after a skin procedure or surgical procedure in a subject comprising: (a) applying to a skin region of the subject a first topical composition comprising a tripeptide-1 and hexapeptide-12 before the skin procedure or the surgical procedure; and (b) applying to the skin region of the subject a second topical composition comprising tripeptide-1, a hexapeptide-12, and a hexapeptide-11, wherein the first topical composition, the second topical composition, or both is administered in an amount sufficient to modulate an expression level of one or more inflammatory or regenerative genes. In one feature, step (b) further comprises applying the first topical composition. In one feature, the first topical composition is administered for at least one week before the surgical procedure. In one feature, the first topical composition is administered for at least two weeks before the surgical procedure. In one feature, the second topical composition is administered for at least two weeks after the surgical procedure. In one feature, the second topical composition is administered for at least ten weeks after the surgical procedure. In one feature, the first topical composition, the second topical composition, or both is administered one, two, three, four, five, or six times a day. In one feature, the first topical composition is administered at least two times a day for at least two weeks before the skin procedure or the surgical procedure and the second topical composition is administered at least two times a day for at least two weeks after the skin procedure or the surgical procedure. In one feature, the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof. In one feature, the one or more inflammatory or regenerative genes encodes a chemokine receptor, a chemokine ligand, an angiotensin receptor, a complement component, a cholinergic receptor, a clusterin, a cytochrome, a fibroblast growth factor, a hepatocyte growth factor, an interleukin receptor, an interleukin, an integrin, a killer cell like lectin receptor, a leukotriene synthase, a lymphocyte antigen, a nuclear factor of activated T-cell, a Nod-like receptor (NLR), a phospholipase, a serpin, a single Ig IL-1-related receptor, a sialic acid binding Ig like lectin, a tachykinin receptor, a TNF receptor, a tyrosylprotein sulfotransferase, or a fragment or variant thereof In one feature, the one or more inflammatory or regenerative genes encodes a chemokine-like receptor, a chemokine ligand, a cluster of differentiation ligand, a cholinergic receptor, a fms related tyrosine kinase ligand, a growth differentiation factor, an interleukin, an interleukin receptor, a lymphocyte antigen, a matrix metallopeptidase, a Nod-like receptor (NLR), a phospholipase, a signal peptide, a CUB domain and EGF like domain, a sialophorin, a transglutaminase, or a fragment or variant thereof. In one feature, the one or more inflammatory or regenerative genes comprises ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In one feature, the administration of the topical composition modulates two or more inflammatory or regenerative genes comprising ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In one feature, the one or more inflammatory or regenerative genes comprises AGTR1, C1R, C3, CCL20, CCRL2, CLU, CYBB, FIGF, IL21R, ITGB2, KLRG1, LTC4S, LY86, NFAM1, NFATC4, NLRP3, PLCB2, SERPINA1, SIGLEC1, or TNFSF13B. In one feature, the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In one feature, the administration of the topical composition modulates two or more inflammatory or regenerative genes comprising AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In one feature, the one or more inflammatory or regenerative genes comprises IL6, PLA2G4C, TNFRSF4, 77VFSFI5, or TPST1. In one feature, the one or more inflammatory or regenerative genes comprises IL6. In one feature, the one or more inflammatory or regenerative genes comprises C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In one feature, the administration of the topical composition modulates two or more inflammatory or regenerative genes comprising C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In one feature, the one or more inflammatory or regenerative genes comprises C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. In one feature, the administration of the topical composition modulates two or more inflammatory or regenerative genes comprising C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. In one feature, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In one feature, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In one feature, the one or more inflammatory or regenerative genes comprises C1R or SCUBE1. In one feature, the one or more inflammatory or regenerative genes comprises TGM2 or PLCB2. In one feature, the expression level is increased by at least 1.25-fold as compared to control. In one feature, the expression level is increased by at least 1.5-fold as compared to control. In one feature, the expression level is increased by at least 2-fold as compared to control. In one feature, the expression level is increased by at least 3-fold as compared to control. In one feature, the control is a skin region that does not receive the first topical composition, the second topical composition, or both. In one feature, the control is a baseline expression level. In one feature, the expression level is increased after at least about 2 weeks. In one feature, the expression level is increased after at least about 4 weeks. In one feature, the methods further comprise subsequent to administration of the topical composition, detecting the expression level of the at least one gene by contacting a sample obtained from the treated skin region of the subject with a probe that recognizes the at least one gene and detect binding between the at least one gene and the probe. In one feature, the methods further comprise before administration of the topical composition, detecting expression level of the one or more inflammatory or regenerative genes comprising ACKR4, AGTR1, C1R, C1S C3, CCL17, CCL19, CCL20, CCL22, CCRL1, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B by contacting a skin sample of the subject with a probe that recognizes the one or more inflammatory or regenerative genes comprising ACKR4, AGTR1, C1R, C1S C3, CCL17, CCL19, CCL20, CCL22, CCR19, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B and detect binding between the gene and the probe. In one feature, the tripeptide-1 comprises palmitoyl tripeptide-1, myristoyl tripeptide-1, or a combination thereof. In one feature, the tripeptide-1 is present at 1-10 ppm. In one feature, the hexapeptide-11 comprises palmitoyl hexapeptide-11, myristoyl hexapeptide-11, or a combination thereof. In one feature, the hexapeptide-11 is present at 0.001-1 ppm. In one feature, the hexapeptide-12 comprises palmitoyl hexapeptide-12, myristoyl hexapeptide-12, or a combination thereof. In one feature, the hexapeptide-12 is present at 0.5-10 ppm. In one feature, the second topical composition further comprises a tetrapeptide. In one feature, the tetrapeptide is tetrapeptide-2. In one feature, the tetrapeptide is present at 1-10 ppm. In one feature, the first topical composition comprises phosphatidylserine and oleuropein. In one feature, the phosphatidylserine is present in a range of about 0.005 weight (wt.) % to about 0.1 wt. %. In one feature, the phosphatidylserine is present at no more than 5.0 wt. %. In one feature, the oleuropein is present at no more than 0.050 wt. %. In one feature, the second topical composition further comprises phosphatidylserine. In one feature, the phosphatidylserine is present in a range of about 0.005 wt. % to about 0.1 wt. %. In one feature, the phosphatidylserine is present at no more than 5.0 wt. %. In one feature, the first topical composition, the second topical composition, or both further comprises ceramide NP, Tremella fuciformis extract, niacinamide, hydrogenated lecithin, C12-16 alcohols, palmitic acid, avocado extract, shea butter, bentonite, phytoene/phytofluene, hydroxymethoxyphenyl decanone, polyholosides, Plantago lanceolata, dill extract, hydrolyzed Candida saitoana extract, Centella asiatica, propanediol, lecithin, Euglena gracilis extract, aqua, caffeine, Glaucium flavum leaf extract, or combinations thereof. In one feature, the first topical composition, the second topical composition, or both is an aqueous topical composition. In one feature, the first topical composition, the second topical composition, or both is an anhydrous topical composition. In one feature, the subject is a human. In one feature, the skin procedure comprises a laser treatment, a chemical peel, microdermabrasion, microneedling, or radiofrequency microneedling. In one feature, the surgical procedure comprise a panniculectomy, liposuction, a lower body lift, brachioplasty, an inner thigh lift, a buttock augmentation, a circumferential body lift, a breast lift, a breast reduction, a breast augmentation, or a labiaplasty. In one feature, the method accelerates the healing process, accelerates clearance of products including lipid particles, accelerates resolution of inflammation stimulates extracellular matrix remodeling, reduces induration, reduces fibrous banding, reduces pain or discomfort, or combinations thereof.

An aspect described herein are methods for improving wound healing in a subject comprising applying to a skin region of the subject a topical composition comprising a tripeptide-1, a hexapeptide-12, and a hexapeptide-11, wherein the topical composition is administered in an amount sufficient to modulate an expression level of one or more inflammatory or regenerative genes, and wherein the topical composition is administered before a procedure, after a procedure, or both, wherein the procedure is a surgical skin removal procedure or a procedure to remove fat and skin. In one feature, the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof. In one feature, the one or more inflammatory or regenerative genes encodes a chemokine receptor, a chemokine ligand, an angiotensin receptor, a complement component, a cholinergic receptor, a clusterin, a cytochrome, a fibroblast growth factor, a hepatocyte growth factor, an interleukin receptor, an interleukin, an integrin, a killer cell like lectin receptor, a leukotriene synthase, a lymphocyte antigen, a nuclear factor of activated T-cell, a Nod-like receptor (NLR), a phospholipase, a serpin, a single Ig IL-1-related receptor, a sialic acid binding Ig like lectin, a tachykinin receptor, a TNF receptor, a tyrosylprotein sulfotransferase, or a fragment or variant thereof. In one feature, the one or more inflammatory or regenerative genes encodes a chemokine-like receptor, a chemokine ligand, a cluster of differentiation ligand, a cholinergic receptor, a fms related tyrosine kinase ligand, a growth differentiation factor, an interleukin, an interleukin receptor, a lymphocyte antigen, a matrix metallopeptidase, a Nod-like receptor (NLR), a phospholipase, a signal peptide, a CUB domain and EGF like domain, a sialophorin, a transglutaminase, or a fragment or variant thereof In one feature, the one or more inflammatory or regenerative genes comprises ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In one feature, the administration of the topical composition modulates two or more inflammatory or regenerative genes comprising ACKR4, AGTR1, C1R, C1S C3, CCL17, CCL20, CCR19, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In one feature, the one or more inflammatory or regenerative genes comprises AGTR1, C1R, C3, CCL20, CCRL2, CLU, CYBB, FIGF, IL21R, ITGB2, KLRG1, LTC4S, LY86, NFAM1, NFATC4, NLRP3, PLCB2, SERPINA1, SIGLEC1, or TNFSF13B. In one feature, the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In one feature, the administration of the topical composition modulates two or more inflammatory or regenerative genes comprising AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In one feature, the one or more inflammatory or regenerative genes comprises IL6, PLA2G4C, TNFRSF4, TNFSF15, or TPST1. In one feature, the one or more inflammatory or regenerative genes comprises IL6. In one feature, the one or more inflammatory or regenerative genes comprises C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In one feature, the administration of the topical composition modulates two or more inflammatory or regenerative genes comprising C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In one feature, the one or more inflammatory or regenerative genes comprises C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. In one feature, the administration of the topical composition modulates two or more inflammatory or regenerative genes comprising C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. In one feature, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In one feature, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In one feature, the one or more inflammatory or regenerative genes comprises C1R or SCUBE1. In one feature, the one or more inflammatory or regenerative genes comprises TGM2 or PLCB2. In one feature, the expression level is increased by at least 1.25-fold as compared to control. In one feature, the expression level is increased by at least 1.5-fold as compared to control. In one feature, the expression level is increased by at least 2-fold as compared to control. In one feature, the expression level is increased by at least 3-fold as compared to control. In one feature, the control is a skin region that does not receive the topical composition. In one feature, the control is a baseline expression level. In one feature, the expression level is increased after at least about 2 weeks. In one feature, the expression level is increased after at least about 4 weeks. In one feature, the methods further comprise subsequent to administration of the topical composition, detecting the expression level of the at least one gene by contacting a sample obtained from the treated skin region of the subject with a probe that recognizes the at least one gene and detect binding between the at least one gene and the probe. In one feature, the methods further comprise before administration of the topical composition, detecting expression level of the one or more inflammatory or regenerative genes comprising ACKR4, AGTR1, C1R, C1S C3, CCL17, CCL19, CCL20, CCL22, CCR19, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B by contacting a skin sample of the subject with a probe that recognizes the one or more inflammatory or regenerative genes comprising ACKR4, AGTR1, C1R, C1S C3, CCL17, CCL19, CCL20, CCL22, CCR19, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSFI3B and detect binding between the gene and the probe. In one feature, the tripeptide-1 comprises palmitoyl tripeptide-1, myristoyl tripeptide-1, or a combination thereof. In one feature, the tripeptide-1 is present at 1-10 ppm. In one feature, the hexapeptide-11 comprises palmitoyl hexapeptide-11, myristoyl hexapeptide-11, or a combination thereof. In one feature, the hexapeptide-11 is present at 0.001-1 ppm In one feature, the hexapeptide-12 comprises palmitoyl hexapeptide-12, myristoyl hexapeptide-12, or a combination thereof. In one feature, the hexapeptide-12 is present at 0.5-10 ppm. In one feature, the methods further comprise a tetrapeptide. In one feature, the tetrapeptide is tetrapeptide-2. In one feature, the tetrapeptide is present at 1-10 ppm. In one feature, the topical composition further comprises phosphatidylserine. In one feature, the phosphatidylserine is present in a range of about 0.005 wt. % to about 0.1 wt. %. In one feature, the phosphatidylserine is present at no more than 5.0 wt. %. In one feature, the methods further comprise ceramide NP, Tremella fuciformis extract, niacinamide, hydrogenated lecithin, C12-16 alcohols, palmitic acid, avocado extract, shea butter, bentonite, phytoene/phytofluene, hydroxymethoxyphenyl decanone, polyholosides, Plantago lanceolata, dill extract, oleuropein, hydrolyzed Candida saitoana extract, Centella asiatica, propanediol, lecithin, Euglena gracilis extract, aqua, caffeine, Glaucium flavum leaf extract, or combinations thereof. In one feature, the topical composition is an aqueous topical composition. In one feature, the topical composition is an anhydrous topical composition. In one feature, the topical composition is administered for at least two weeks before the procedure. In one feature, the topical composition is administered for at least two weeks after the procedure. In one feature, the topical composition is administered one, two three, four, five, or six times a day. In one feature, the topical composition is administered one, two three, four, five, or six times a day for at least two weeks before the procedure and at least two weeks after the procedure. In one feature, the method accelerates the healing process, accelerates clearance of products including lipid particles, accelerates resolution of inflammation stimulates extracellular matrix remodeling, reduces induration, reduces fibrous banding, reduces pain or discomfort, or combinations thereof. In one feature, the subject is a human.

### BRIEF DESCRIPTION OF THE DRAWINGS

FIG. 1 depicts a Venn diagram comparing upregulated genes in the 2-week untreated versus the 2-week treated groups. The numbers are based on the genes that were significantly upregulated (≥1.5-fold) in comparison to the pre-treatment biopsies. 2W UT is 2-week untreated group; 2W T is 2-week treated group.

FIGS. 2A-2B depict protein:protein interaction networks for the 2 week groups. The String database was used to compare the interaction networks for the 2-week untreated group (FIG. 2A) and the 2-week treated group (FIG. 2B).

FIG. 3 Venn diagram comparing the upregulated genes in the 2-week treated versus the 4-week treated groups. The numbers are based on the genes that were significantly upregulated (≥1.5-fold) in comparison to the pre-treatment biopsies. 2W T is 2-week treated group; 4W T is 4-week treated group.

FIGS. 4A-4B depict protein:protein interaction networks for the 4 week groups. The String database was used to compare the interaction networks for the 4-week untreated group (FIG. 4A) and the 4-week treated group (FIG. 4B).

FIG. 5A depicts a graph of skin induration assessments made using a SkinFibroMeter, which assesses induration in absolute units of stiffness. An increased value over the baseline reading is indicative of stiffness/induration. The data are presented at the percent increase over baseline for the 1-week and 2-week time points for both the treated and untreated sides. The data are represented as the mean±standard error the mean. The *indicates p<0.05 (n=6).

FIG. 5B depicts a graph of skin induration assessments using a blinded investigator assessment. The level of induration was assessed using a graded scale as follows: 0 is none; 1 is barely perceptible; 2 is slight; 3 is moderate; and 4 is severe. The data represented are the meant standard error the mean from the 2-week and 4-week follow ups, which correspond to the biopsy time points. The *indicates p<0.05 (n=6).

FIG. 6A depicts a graph of a blinded investigator assessment of induration, edema, skin discoloration, ecchymosis, subcutaneous banding and pain one week after liposuction. Each assessment was made using a 0-4-point scale (0=none, 1=barely perceptible, 2=slight, 3=moderate and 4=severe). The VAS pain scale was also used to assess pain (0-10 scale).

FIG. 6B depicts a graph of a blinded investigator assessment of induration, edema, skin discoloration, ecchymosis, subcutaneous banding and pain two weeks after liposuction. Each assessment was made using a 0-4-point scale (0=none, 1=barely perceptible, 2=slight, 3=moderate and 4=severe). The VAS pain scale was also used to assess pain (0-10 scale).

FIG. 6C depicts a graph of a blinded investigator assessment of induration, edema, skin discoloration, ecchymosis, subcutaneous banding and pain four weeks after liposuction. Each assessment was made using a 0-4—point scale (0=none, 1=barely perceptible, 2=slight, 3=moderate and 4=severe). The VAS pain scale was also used to assess pain (0-10 scale).

FIG. 7 depicts a graph of mean SkinFibrometer measurement changes from baseline. The mean change was calculated for each leg at every visit.

FIG. 8 depicts an ultrasound image used to compare fluid disruption into dermal segment representing induration and edema showing less disruption of dermal subcutaneous boundary on regenerating body complex product (RBP) side. To measure the transition from the dermal to subcutaneous tissue the difference between the dermal and subcutaneous intensity analysis was calculated. RDIS is relative dispersion, a measure of relative variation.

FIG. 9 depicts a Herovici Stain at 40X of Subject 4 following administration of comparator product (left panel images) and regenerating skin nectar (RSN) and regenerating body complex product (RBP) (right panel images) at baseline (top row), 2 weeks (middle row) and 4 weeks (bottom row).

FIG. 10 depicts a graph of mean participant assessments. At each visit following the liposuction procedure each participant was given a questionnaire to assess each leg on a 0-3—point scale (0=none, 1=mild, 2=moderate and 3=severe).

FIG. 11 depicts a graph of mean investigator assessment scores for edema in participants at one week, two weeks, and four weeks post-liposuction in patients when they received one versus two treatments. Each assessment was graded using a (0-4) point scale. The VAS, a (0-10) point scale, was used to score pain. Submental fullness (CR-SFRS) was graded at every visit using the submental fat rating scale, (0-4) point scale.

FIG. 12 depicts a graph of mean investigator assessment scores for induration in participants at one week, two weeks, and four weeks post-liposuction in patients when they received one versus two treatments. Each assessment was graded using a (0-4) point scale. The VAS, a (0-10) point scale, was used to score pain. Submental fullness (CR-SFRS)₇was graded at every visit using the submental fat rating scale, (0-4) point scale.

FIG. 13 depicts a graph of mean change in submental fat rating scale at four weeks post-liposuction (CR-SFRS).

FIG. 14 depicts SkinFibrometer mean change from baseline in the right and left legs of patients when they received one treatment, two treatments, or the bland moisturizer.

FIG. 15 depicts images of Patient 4 at baseline, 2 weeks after one treatment with TF, and two weeks after two treatments with bland moisturizer.

FIG. 16 depicts images of Patient 10 at baseline, two weeks after one treatment with RBP, and two weeks after two treatments with RBP.

FIG. 17 depicts a graph indicating percent of adverse reactions, particularly soreness and tenderness, among patients.

### DETAILED DESCRIPTION

The following description and examples illustrate a preferred embodiment of the present disclosure in detail. Those of skill in the art will recognize that there are numerous variations and modifications of this disclosure that are encompassed by its scope. Accordingly, the description of a preferred embodiment should not be deemed to limit the scope of the present disclosure. Definitions

The terms “pharmaceutically acceptable salts” and “a pharmaceutically acceptable salt thereof” as used herein are broad terms, and are to be given their ordinary and customary meaning to a person of ordinary skill in the art (and are not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Compositions

The compositions described herein can modulate inflammation and wound healing. In some embodiments, the compositions described herein modulate inflammatory or regenerative genes. In some embodiments, the compositions described herein accelerate the healing process, accelerate clearance of products including lipid particles, induce inflammatory or regenerative gene expression, accelerate resolution of inflammation, stimulate extracellular matrix remodeling, reduce induration, reduce fibrous banding, reduce pain or discomfort, or combinations thereof.

Peptides

The peptide combinations of the embodiments can be employed in various types of compositions. Topical compositions including a dipeptide, tripeptide, or tetrapeptide, and a pentapeptide, hexapeptide, or heptapeptide in combination with at least one excipient, are provided. In some embodiments, topical compositions comprise one or more tripeptides, one or more tetrapeptides, and one or more hexapeptides. In some embodiments, a tripeptide of the one or more tripeptides is tripeptide-1. In some embodiments, a tetrapeptide of the one or more tetrapeptides is tetrapeptide-2. In some embodiments, a hexapeptide of the one or more hexapeptides is hexapeptide-12. In some embodiments, a hexapeptide of the one or more hexapeptides is hexapeptide-11. In some embodiments, the topical composition comprises tripeptide-1, tetrapeptide-2, hexapeptide-12, and hexapeptide-11. In some embodiments, the composition comprises a tripeptide-1, a hexapeptide-12 and a hexapeptide-11. In some embodiments, the topical composition further comprises a tetrapeptide. In some embodiments, the tetrapeptide is tetrapeptide-2. In some embodiments, the topical composition comprises tripeptide-1, tetrapeptide-2, and hexapeptide-12.

In some embodiments, the topical compositions comprise from about 0.001 wt. % or less to about 50 wt. % or more of active ingredient, such as the peptides, preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %. In some embodiments, the active ingredient is provided at least or about 0.001%, 0.005%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the active ingredient is provided in a range of about 0.25% to about 10%, about 0.5% to about 8%, about 0.75% to about 6%, or about 1% to about 4% by weight. In some embodiments, the active ingredient is provided in a range of about 0.001% to about 6%, about 0.002% to about 4%, about 0.01% to about 3%, or about 0.02% to about 2% by weight.

Compositions comprising a combination of two or more peptides are provided for modulating inflammation and wound healing. In a topical composition comprising the two peptide combination, a first peptide (e.g., hexapeptide) is present in the composition in pure for or in a form of a carrier containing the peptide, e.g., 0.001, 0.01, 0.1, 1, 10, 50 ppm or less to 100, 1000, 5000, 10000, 50000, 100000, 500000 ppm or more, e.g., 100 ppm of the peptide. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 10 wt. % of the first peptide. The second peptide (e.g., tripeptide) is present in the topical formulation composition in pure form or in a form of a carrier containing the peptide, e.g., 0.001, 0.01, 0.1, 1, 10, 50 ppm or less to 100, 1000, 5000, 10000, 50000, 100000, 500000 ppm or more, e.g., 100 ppm of the peptide, or any other suitable amount. The topical formulation can contain from 0.01 wt. % or less (e.g., 0.001 wt. %) to 10 wt. % or more, e.g., 0.01 wt. % to 0.02 wt. %, 0.03 wt. %, 0.04 wt. %, 0.05 wt. %, 0.1 wt. %, 1 wt. % to 5 wt. % or 20 wt. % of the second peptide. The amount of peptide in the base can be adjusted up or down.

Compositions as described herein, in some embodiments, comprise one or more peptides. In some embodiments, the one or more peptides is provided at least or about 0.001%, 0.005%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the one or more peptides is provided in a range of about 0.25% to about 10%, about 0.5% to about 8%, about 0.75% to about 6%, or about 1% to about 4% by weight. In some embodiments, the one or more peptides is provided in a range of about 0.001% to about 6%, about 0.002% to about 4%, about 0.01% to about 3%, or about 0.02% to about 2% by weight.

In some embodiments, the peptide of the one or more peptides is tripeptide-1, tetrapeptide-2, hexapeptide-12, or hexapeptide-11. In some embodiments, the tripeptide-1 is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the tripeptide-1 is provided in a range of about 0.25% to about 10%, about 0.5% to about 8%, about 0.75% to about 6%, or about 1% to about 4% by weight. In some embodiments, the tetrapeptide-2 is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the tetrapeptide-2 is provided in a range of about 0.25% to about 10%, about 0.5% to about 8%, about 0.75% to about 6%, or about 1% to about 4% by weight. In some embodiments, the hexapeptide-12 is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hexapeptide-12 is provided in a range of about 0.25% to about 10%, about 0.5% to about 8%, about 0.75% to about 6%, or about 1% to about 4% by weight. In some embodiments, the hexapeptide-11 is provided at least or about 0.001%, 0.005%, 0.01%, 0.02%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hexapeptide-11 is provided in a range of about 0.25% to about 10%, about 0.5% to about 8%, about 0.75% to about 6%, or about 1% to about 4% by weight. In some embodiments, the hexapeptide-11 is provided in a range of about 0.001% to about 6%, about 0.002% to about 4%, about 0.01% to about 3%, or about 0.02% to about 2%. In some embodiments, the hexapeptide-11 is provided in a range of about 0.005% to about 0.02% by weight.

In example embodiments, a weight ratio for the first peptide to the second peptide in a topical composition is 1 part first peptide to 0.2 to 10 parts second peptide, or 1 to 10 parts second peptide, or 1 to 8 parts second peptide, or 1 to 5.5 parts second peptide. The following nomenclature is employed herein to refer to various amino acids: Alanine (also referred to herein as “Ala” or “A”), Arginine (also referred to herein as “Arg” or “R”), Asparagine (also referred to herein as “Asn” or “N”), Aspartic acid (also referred to herein as “Asp” or “D”), Cysteine (also referred to herein as “Cys” or “C”), Glutamic acid (also referred to herein as “Glu” or “E”), Glutamine (also referred to herein as “Gln” or “Q”), Glycine (also referred to herein as “Gly” or “G”), Histidine (also referred to herein as “His” or “H”), Isoleucine (also referred to herein as “Ile” or “I”), Leucine (also referred to herein as “Leu” or “L”), Lysine (also referred to herein as “Lys” or “K”), Methionine (also referred to herein as “Met” or “M”), Phenylalanine (also referred to herein as “Phe” or “F”), Proline (also referred to herein as “Pro” or “P”), Serine (also referred to herein as “Ser” or “S”), Threonine (also referred to herein as “Thr” or “T”), Tryptophan (also referred to herein as “Trp” or “W”), Tyrosine (also referred to herein as “Tyr” or “Y”), Valine (also referred to herein as “Val” or “V”).

In some embodiments, the first peptide is a dipeptide. Suitable dipeptides include but are not limited to those having the following sequence of amino acids: KK, KP, CK, KC, KT, DF, NF, VW, YR, or TT. In some embodiments, the dipeptide has the following amino acid sequence: KV. In other embodiments, the first peptide is a tripeptide. Suitable tripeptides include but are not limited to those having the following sequence of amino acids: HGG, RKR, GHK, GKH, GGH, GHG, KFK, or KPK. In some embodiments, the tripeptide has the following amino acid sequence: KVK. In some embodiments, the first peptide is a tetrapeptide. Suitable tetrapeptides include but are not limited to those having the following sequence of amino acids: GQPR, KTFK, AQTR, or RSRK. In some embodiments, the tetrapeptide has the following sequence of amino acids: KDVY. In some embodiments, the second peptide is a pentapeptide. Suitable pentapeptides include but are not limited to those having the following sequence of amino acids: KTTKS, YGGFX, or KLAAK. In some embodiments, the second peptide is a hexapeptide. Suitable hexapeptides include but are not limited to those having the following sequence of amino acids: VGVAPG or GKTTKS. In some embodiments, the hexapeptide has the following sequence of amino acids: FVAPFP. In some embodiments, the second peptide is a heptapeptide. Suitable heptapeptides include but are not limited to one having an amino acid sequence RGYYLLE, or Heptapeptide-6 (a pro-sirtuin peptide). The compositions may include two or more peptides, e.g., two dipeptides and one pentapeptide; one tripeptide and one hexapeptide; one dipeptide, one tripeptide, and one heptapeptide, or the like, provided that the composition contains at least one dipeptide, tripeptide, or tetrapeptide and at least one pentapeptide, hexapeptide, or heptapeptide. In some embodiments, the compositions comprise one or more tripeptides, one or more tetrapeptides, and one or more hexapeptides. In some embodiments, a tripeptide of the one or more tripeptides is tripeptide-1. In some embodiments, a tetrapeptide of the one or more tetrapeptides is tetrapeptide-2. In some embodiments, a hexapeptide of the one or more hexapeptides is hexapeptide-12. In some embodiments, a hexapeptide of the one or more hexapeptides is hexapeptide-11. In some embodiments, the compositions comprise tripeptide-1, tetrapeptide-2, hexapeptide-12, and hexapeptide-11. In some embodiments, the compositions comprise tripeptide-1, tetrapeptide-2, and hexapeptide-12.

The peptide can be functionalized. For example, the peptide can be functionalized with a fatty acid, e.g., myristoleic acid, palmitoleic acid, sapienic acid, oleic acid, elaidic acid, vaccenic acid, linoleic acid, linoelaidic acid, a-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. Examples include palmitoyl hexapeptide-12 (Pal-VGVAPG), palmitoyl tripeptide-1 (Pal-GHK), myristoyl hexapeptide-12 (Myr-VGVAPG), myristoyl tripeptide-1 (Myr-GHK). Palmitoyl or myristoyl functionalization can be desirable in certain embodiments as it exhibits enhanced penetration when compared to other fatty acids. In some embodiments, the tripeptide-1 comprises palmitoyl-tripeptide-1, myristoyl tripeptide-1, or a combination thereof. In some embodiments, the hexapeptide-11 comprises palmitoyl-hexapeptide-11, myristoyl hexapeptide-11, or a combination thereof. In some embodiments, the tripeptide-1 comprises palmitoyl-tripeptide-1, myristoyl tripeptide-1, or a combination thereof In some embodiments, the hexapeptide-12 comprises palmitoyl hexapeptide-12, myristoyl hexapeptide-12, or a combination thereof. In some embodiments, the peptide is functionalized with a chemical group. For example, the peptide is functionalized with acetyl. Examples include acetyl tetrapeptide-2.

Some embodiments of the methods and compositions provided herein include as a first peptide glycine-histidine-lysine (GHK). GHK is a peptide sequence that is rarely found in the class of proteins in general, but is frequently found in extracellular matrix proteins. The small size of GHK permits it to approach membrane receptors far more easily than larger peptides. Further, its unique, copper-binding structure enhances copper transport into and out of cells and promotes wound healing through several different but related pathways. Due to its strong copper binding structure, GHK can be provided in the form of GHK-Cu (copper-bound GHK form).

GHK acts as an anti-inflammatory (see, e.g., Pickart, L., The human tri-peptide GHK and tissue remodeling, J. Biomater. Sci. Polymer Edn. 2008, Vol. 19, pp. 969-988, 972-973; Pickart et al., The Human Tripeptide GHK-CU in Prevention of Oxidative Stress and Degenerative Conditions of Aging: Implications for Cognitive Health, Oxid. Med. Cell Longev. 2012, Vol. 2012, pp. 1-8, 3) and an antioxidant. GHK acts to promote wound healing by suppressing the “acute phase response” that can produce both inflammation and induce scarring. This biological response prevents the invasion of bacteria, facilitates the arrival of immune cells, stems bleeding, and provides a covering for the wounded area. GHK-Cu also suppresses the acute phase response by inhibiting the production of molecules called cytokines. Cytokines are immune cell signaling molecules that attract immune cells and that trigger the production of other molecules that promote inflammation and fibrosis (leading to the creation of scar tissue). In particular, GHK suppresses the production of cytokines including tumor necrosis factor-alpha (TNFα), interleukin-1 (IL-1), interleukin-6 (IL-6), and transforming growth factor-beta-1 (TGF-β1), a few of the key drivers of inflammation and apoptotic cell death in the wound region. As TGF-β1 is an important component for the continuation of the acute phase response, GHK's suppression of TGF-β1 also acts to shorten the duration of the acute phase response once it has begun. GHK acts as an antioxidant by blocking ferritin's release of oxidizing iron, preventing further inflammation or microbial infection (as invading microbes need iron to survive).

GHK also stimulates blood vessel growth, increases collagen production, and regenerates the extracellular matrix. GHK acts as an attractant for cells vital to the regeneration of damaged tissues such as capillary cells that rebuild blood vessels. It also upregulates the production of a variety of enzymes that remove damaged proteins while also rebuilding the extracellular matrix (ECM), a key external scaffold that is important for intercellular communication and support. In particular, GHK's induces the production of messenger RNAs (mRNAs) necessary for the regeneration of the ECM, namely collagen, proteoglycans, glycosaminoglycans, chondroitin sulfate, and dermatan sulfate. GHK' s induction of increased collagen production also plays a key role in enhancing skin regrowth. GHK further stimulates blood flow into damaged tissues through three processes: angiogenesis, anti-coagulation and vascular dilation. First, GHK induces angiogenesis or new blood vessel formation by increasing the production of growth factor proteins necessary for angiogenesis such as basic fibroblast growth factor (BFGF) and vascular endothelial growth factor (VEGF). Second, GHK increases blood flow to the wounded area by expanding the number of red blood cells (via growth in erythropoietin production) and by anti-coagulatory effects such as downregulating the blood clotting molecule thromboxane. Third, GHK facilitates vascular dilation through binding to the vasoconstriction protein angiotensin II, preventing angiotensin from constricting blood vessels and reducing blood flow.

GHK promotes stem cell proliferation (see, e.g., Ito et al., Is the Hair Follicle Necessary for Normal Wound Healing, J. Invest. Dermatol. 2008, Vol. 128, pp. 1059-1061, 1059). Wound healing studies have demonstrated that the addition of GHK-Cu greatly enlarged the production of hair follicles near the wound periphery in experiments with mice. Dermal hair follicles are a significant source of stem cells that are essential for dermal healing. Research into dermal hair follicles have demonstrated that hair-bearing areas tend to heal more quickly and that cells from various portions of the follicle may contribute to both dermal cell and epithelial cell replacement as well.

Thus, by decreasing inflammation, acting as an antioxidant, stimulating growth of new blood vessels, regenerating the extracellular matrix, enhancing collagen production, and by promoting stem cell proliferation, GHK can greatly enhance skin regeneration and promote wound healing.

Some embodiments of the methods and compositions provided herein include as a second peptide valine-glycine-valine-alanine-proline-glycine (VGVAPG). VGVAPG is a hexapeptide that is derived from the elastin protein (see, e.g., Blanchevoye et al., Interaction between the Elastin Peptide VGVAPG and Human Elastin Binding Protein, J. Biol. Chem. 2012, Vol. 288, pp. 1317-1328, 1317-1318) (“VGVAPG” disclosed as SEQ ID NO: 9). Elastin is a protein found in connective tissue (e.g. skin) that is necessary for tissues to return to their original shape and size after undergoing temporary expansion or contraction. Due to the importance of elastin in providing elasticity and resilience, elastin plays a significant role in skin cell resistance to injury and recovery from injury. The ability of skin to return to its original form after undergoing stretching or pulling relies on cross-linked elastin proteins (tropoelastin proteins in humans) that work to form “elastic fibers.” The disruption of the elastic fiber system in healing wounds has been strongly linked to the production of scar tissue (see, e.g., Rnjak-Kovacina et al., Severe Burn Injuries and the Role of Elastin in the Design of Dermal Substitutes, Tissue Eng. Part B. Rev. 2011, pp. 81-91, 85-86). Because of these properties and others, elastin is a key component in the effective wound healing process.

VGVAPG plays a role in facilitating elastin's ability to prevent skin injury and to promote skin regeneration (see, e.g., Floquet et al., Structural Characterization of VGVAPG, an Elastin-Derived Peptide, Biopolymers (Peptide Science) 2004, Vol. 76, 266-280, 267) (“VGVAPG” disclosed as SEQ ID NO: 9). First, it has been shown to demonstrate the ability to attract monocytes and fibroblasts (see, e.g., Senior et al., Val-Gly-Val-Ala-Pro-Gly, a Repeating Peptide in Elastin, Is Chemotactic for Fibroblasts and Monocytes, J. Cell Biol. 1984, Vol. 99, pp. 870-874, 870) (“Val-Gly-Val-Ala-Pro-Gly” disclosed as SEQ ID NO: 9), monocytes being essential for fighting off infection and fibroblasts being necessary for collagen production (the most abundant protein in skin) and for the regeneration of the extracellular matrix. Second, VGVAPG provides a binding site for elastin-binding protein, a permanent component of mature elastic fibers. Third, VGVAPG provides a binding site for elastin and extracellular matrix degradation enzymes such as matrix metalloproteinases (MMPs), which facilitate the replacement and regeneration of elastic fibers and extracellular matrix proteins.

The tripeptide and hexapeptide work synergistically to promote skin regeneration and wound healing through the attraction of healing cells, increased production of elastin and collagen, enhanced fibroblast proliferation, antioxidant behavior (preventing the release of oxidizing iron), and inducing the regeneration of the extracellular matrix. As a result, the combination of the two peptides exhibits synergistic, superior performance well beyond that expected for either of the two peptides alone.

Tripeptides promote skin regeneration through increased collagen and elastin synthesis, blocking ferritin release of oxidized iron, attracting healing cells such as capillary cells and macrophages, and through re-establishing new blood flow to the injury site. The tripeptide functions as an anti-oxidant, stimulates collagen, elastin, and hyaluronic acid. It is formulated to penetrate stratum corneum. In the extracellular matrix (ECM), it is an anti-oxidant, attracts capillaries and macrophages, which facilitates wound healing. In the cell, it decreases inflammatory cytokines, increases collagen, elastin, dermal stem cell proliferation, and hyaluronic acid.

Hexapeptides promote skin regeneration and wound healing through the induction of elastin and collagen production, fibroblast proliferation, regeneration of the extracellular matrix, and fibroblast keratinocyte mobility. The hexapeptide is formulated to penetrate the stratum corneum, and mimics the elastin binding sequence, to stimulate elastin. It binds specifically to EBP receptors on fibroblasts and keratinocytes. The binding initiates intracellular signal transduction. Hexapeptides suitable for use include Hexapeptide-12 and Hexapeptide-11. Hexapeptide-11 has the sequence: Hex-11 (Phe-Val-Ala-Pro-Phe-Pro (FVAPFP). Hexapeptide-12 has the sequence: VGVAPG.

In topical compositions, the tripeptide is typically present in an amount of from about 0.1 ppm or less to about 10, 100, 200, 300, 400, or 500 ppm or more, e.g., 1 ppm to 10 ppm. In some embodiments, the tripeptide is present in an amount from at least about 0.01, 0.05, 0.1, 0.5, 1, 2.5, 5, 7.5, 10, 12.5, 15, 15.5, 20, or more than 20 ppm. In some embodiments, the tripeptide is present in an amount from no more than about 0.01, 0.05, 0.1, 0.5, 1, 2.5, 5, 7.5, 10, 12.5, 15, 15.5, or 20 ppm. In some embodiments, the tripeptide is present at about 1 ppm to about 10 ppm. In some embodiments, the tripeptide is tripeptide-1.

In topical compositions, the hexapeptide is typically present in an amount of from about 0.001 ppm or less to about 0.01, 0.1, 0.5, 1 100, 200, 300, 400, or 500 ppm or more, e.g., 0.001 to 10 ppm. In some embodiments, the hexapeptide is present in an amount from at least about 0.001, 0.05, 0.01, 0.05, 0.1, 0.5, 1, 2.5, 5, 7.5, 10, 12.5, 15, 15.5, 20, or more than 20 ppm. In some embodiments, the hexapeptide is present in an amount from no more than about 0.001, 0.05, 0.01, 0.05, 0.1, 0.5, 1, 2.5, 5, 7.5, 10, 12.5, 15, 15.5, or 20 ppm. In some embodiments, the hexapeptide is present at about 0.001 ppm to about 1 ppm. In some embodiments, the hexapeptide is present at about 0.5 to about 10 ppm. In some embodiments, the hexapeptide is hexapeptide-12. In some embodiments, the hexapeptide is hexapeptide-11.

In topical compositions, the tetrapeptide is typically present in an amount of from about 0.1 ppm or less to about 10, 100, 200, 300, 400, or 500 ppm or more, e.g., 1 ppm to 10 ppm. In some embodiments, the tetrapeptide is present in an amount from at least about 0.01, 0.05, 0.1, 0.5, 1, 2.5, 5, 7.5, 10, 12.5, 15, 15.5, 20, or more than 20 ppm. In some embodiments, the tetrapeptide is present in an amount from no more than about 0.01, 0.05, 0.1, 0.5, 1, 2.5, 5, 7.5, 10, 12.5, 15, 15.5, or 20 ppm. In some embodiments, the tetrapeptide is present at about 1 ppm to about 10 ppm. In some embodiments, the tetrapeptide is tetrapeptide-2.

The peptides can advantageously be provided in a base for suitable for combining with other components of a topical composition. The base can include one or more components such as a thickener/binding agent (e.g., pentaerythrityl tetraisostearate), an emollient/dispersing agent (e.g., caprylic/capric triglyceride), a solvent (e.g., propylene carbonate), and/or a rheology modifier/antisettling agent (e.g., disteardimonium hectorite).

Oleuropein

In some embodiments, polyphenols such as oleuropein may be added to the compositions. Oleuropein is a polyphenol isolated from olive leaves (see e.g. Omar SH. Oleuropein in olive and its pharmacological effects. Sci Pharm 2010; 78(2): 133-54; Al-Rimawi F, Yateem H, Afaneh I. Formulation and evaluation of a moisturizing day cream containing olive leaves extract. International Journal of Development Research 2014; 4(10): 1996-2000; Kontogianni V G, Charisiadis P, Margianni E, Lamari F N, Gerothanassis I P, Tzakos A G. Olive leaf extracts are a natural source of advanced glycation end product inhibitors. Journal of medicinal food 2013; 16(9): 817-22). Oleuropein demonstrates major anti-inflammatory effects by inhibiting lipoxygenase activity and the production of leukotriene. More particularly researchers have demonstrated that oleuropein enhances proteasome activities in vitro more effectively than other known chemical activators, possibly through conformational changes of the proteasome. In this regard, it decreases reactive oxygen species (ROS), reduces the amount of oxidized proteins through increased proteasome-mediated degradation through increased proteasome -mediated degradation and autophagic pathways, and retains proteasome function during replicative senescence.

In some embodiments, formulations as described herein comprise oleuropein. In some embodiments, the oleuropein is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the oleuropein is provided in a range of about 0.001% to about 6%, about 0.002% to about 4%, about 0.01% to about 3%, about 0.02% to about 2%, or about 0.01% to about 0.05% by weight. In some embodiments, the oleuropein is provided at about 0.010% by weight. In some embodiments, the oleuropein is provided at about 0.020% by weight. In some embodiments, the oleuropein is provided at about 0.050% by weight.

Phosphatidylserine

In certain embodiments, phospholipids such as phosphatidylserine (PS), a highly enriched membrane phospholipid component, may be added. Phosphatidylserine has been known to have several physiological roles, such as activating signaling enzymes and antioxidant activity (see e.g. Draelos, Z., Pugliese, P. Glycation and Skin Aging: A Review. Cosmetics & Toiletries Magazine 2011; June 2011: 1-6; Lee, S., Yang, J., Park Y., et al. Protective effect and mechanism of phosphatidylserine in UVB-induced human dermal fibroblasts. European Journal of Lipid Science and Technology 2013; 115(7): 783-90; He, M., Kubo, H., Morimoto, K., et al. Receptor for advanced glycation end products binds to phosphatidylserine and assists in the clearance of apoptotic cells. EMBO reports 2011; 12(4): 358-64). It has been found to decrease MMP-1 in a dose dependent manner, to increase procollagen formation and may act as a substrate for AGE targets thus reducing the damage from glycation effects. Clearance of apoptotic cells is necessary for tissue development, homeostasis, and resolution of inflammation. Phosphatidylserine provides an “eat me” signal on the cell surface, and phagocytes recognize the signal using specific receptors such as the receptor of advanced glycation end-products (RAGE). This then binds to PS and assists in the clearance of apoptotic cells and end products of AGE.

In some embodiments, formulations as described herein comprise phosphatidylserine. In some embodiments, the phosphatidylserine is provided at least or about 0.001%, 0.002%, 0.005%, 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the phosphatidylserine is provided in a range of about 0.001% to about 6%, about 0.002% to about 4%, about 0.01% to about 3%, about 0.02% to about 2%, or about 0.25% to about 1% by weight. In some embodiments, the phosphatidylserine is provided at about 0.05% by weight. In some embodiments, the phosphatidylserine is provided at about 0.25% by weight. In some embodiments, the phosphatidylserine is provided at about 1% by weight.

Phosphatidylserine can advantageously be employed in compositions for preconditioning the skin in advance of procedures as described herein.

Candida Saitoana

With respect to hydrolyzed Candida saitoana extract, in order to maintain their homeostasis, cells eliminate various accumulated and degraded components. Autophagy, which was recently discovered in skin, stands out today as a powerful mechanism, essential for detoxifying cells and guaranteeing their proper functioning, thereby limiting the senescence. This extract is a purified α-glucan active ingredient, which detoxifies cells by removing altered cell components (oxidized proteins and peroxidized lipids) that saturate them and blocks the accumulation of lipofuscin aggregates, a true marker of aging. See Product monograph: Silab 2013.

In some embodiments, formulations as described herein comprise hydrolyzed Candida saitoana extract. In some embodiments, the hydrolyzed Candida saitoana extract is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hydrolyzed Candida saitoana extract is provided in a range of about 0.25% to about 10%, about 0.5% to about 8%, about 0.75% to about 6%, or about 1% to about 4% by weight. In some embodiments, the hydrolyzed Candida saitoana extract is provided at about 3.0% by weight.

Plantago Lanceolata

With respect to Plantago lanceolata, it inhibits micro RNA inhibition of fibroblast function, reversing cellular senescence, thus increasing collagen, laminin, elastin and decreasing MMP-1. See Kovac I, Durkac J, Holly M, et al. Plantago lanceolata L. water extract induces transition of fibroblasts into myofibroblasts and increases tensile strength of healing skin wounds. J Pharm Pharmacol 2015; 67(1): 117-25, and Debacker A, Lavaissière L, Ringenbach C, Mondon P, Dal Toso R. Controlling MicroRNAs to Fight Skin Senescence. Cosmetics & Toiletries 2016; Feb 4, 2016: 1-6. Small endogenous noncoding RNAs named microRNA (miRNA) bind to partially complementary sequences of their target messenger RNA (mRNA) and repress or degrade the mRNA, which cause gene inactivation or gene silencing. It appears that collagen I, Collagen IV and elastin are partially controlled by several microRNAs, and when these microRNAs are limited, it helps to boost collagen and elastin synthesis to improve the quality of the dermis. Plantago lanceolata extract was found to reduce the levels of expression of miRNAs controlling the synthesis of collagens and elastin increasing their production and reducing the fibroblast progression toward senescence. Additional in vivo studies demonstrated increased viscoelastic properties with increases in firmness of 30.9% and elasticity of 22.6%, after one month of product application (p<0.01) to the skin.

In some embodiments, formulations as described herein comprise Plantago lanceolata. In some embodiments, the Plantago lanceolata is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Plantago lanceolata is provided in a range of about 0.25% to about 10%, about 0.5% to about 8%, about 0.75% to about 6%, or about 1% to about 4% by weight. In some embodiments, the Plantago lanceolata is provided at about 2.0% by weight.

Increasing Elastin Production and Functionality; Increasing Lipolysis—Tightening and Lipid Digestion

Acetyl Tetrapeptide-2 stimulates LOXL1 (Lysyl oxidase like enzyme 1), which cross links elastin components; binds tropoelastin (TE); builds elastin; and increases FBLNS (Fibulin 5), which binds TE to integrin to fibroblast stimulating fibroblast to produce elastin. Palmitoyl Tripeptide-1 provides collagen and elastin stimulation, ECM recycling, anti-inflammation, and with Palmitoyl Hexapeptide-12, an elastin binding protein, draws in newly produced elastin. Dill extract (Anethum graveolens extract) stimulates LOXL reinduction encouraging elastin formation. Avocado extract, shea butter, and bentonite, in some embodiments, provide tightening, elastase inhibition inhibits elastin breakdown and encourages some fat breakdown and turnover; it also aids in stretch mark alleviation.

In some embodiments, avocado extract is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, avocado extract is provided in a range of about 0.01% to about 5%, about 0.02% to about 4%, 0.05% to about 3%, or about 0.1% to about 2% by weight. In some embodiments, shea butter is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, shea butter is provided in a range of about 0.01% to about 5%, about 0.02% to about 4%, 0.05% to about 3%, or about 0.1% to about 2% by weight. In some embodiments, bentonite is provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, bentonite is provided in a range of about 0.01% to about 5%, about 0.02% to about 4%, 0.05% to about 3%, or about 0.1% to about 2% by weight. In some embodiments, avocado extract, shea butter, and bentonite are provided at least or about 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, avocado extract, shea butter, and bentonite are provided in a range of about 0.01% to about 5%, about 0.02% to about 4%, 0.05% to about 3%, about 0.1% to about 2%, or about 0.25% to about 2% by weight. In some embodiments, avocado extract, shea butter, and bentonite are provided at about 0.5% by weight. In some embodiments, avocado extract, shea butter, and bentonite are provided at about 1.0% by weight.

With respect to elastin, it is an assembly of microfibrils and tropoelastin (or soluble elastin). Elastin fibers are formed first by the synthesis of fibrillin microfibers which intertwine and then associate with tropoelastin (TE) protein molecules. TE molecules are bound together and cross linked together with fibrillin fibers by lysyl oxidase like enzyme 1 (LOXL1), a key player regulating the assembly of these two elements — this complex is then presented to the fibroblast by Fibulin 5 (FBLNS) which connects the complex to integrins that connect to the fibroblast. See Ashcroft et al., “Age-related changes in temporal and spatial distributions of fibrillin and elastin mRNAs and proteins in acute cutaneous wounds of healthy humans”, J. Pathology 1997; 183:80-9, Cenizo V, André V, Reymermier C, Sommer P, Damour O, E. P. LOXL as a target to increase the elastin content in adult skin: a dill extract induces the LOXL gene expression. Experimental Dermatology 2006; 15: 574-81, and Noblesse E, Cenizo V, Bouez C, et al. Lysyl oxidase-like and lysyl oxidase are present in the dermis and epidermis of a skin equivalent and in human skin and are associated to elastic fibers. J Invest Dermatol 2004; 122(3): 621-30.

With respect to acetyl tetrapeptide-2, it increases FBLNS and LOXL1 protein levels, thereby increasing elastin synthesis. It also upregulates genes related to Collagen 1 synthesis. In vivo, it has shown to reduce parameters linked to skin flaccidity and dermal disorganization. See Product monograph: Uplevity™ Lipotec. June 2013.

With respect to TriHex (Palmitoyl tripeptide 1 and Palmitoyl hexapeptide 12), it clears the extracellular matrix of aggregated fragmented collagen and elastin and then stimulate increased new collagen and elastin production. See Widgerow A D, Fabi S G, Palestine R F, et al. Extracellular Matrix Modulation: Optimizing Skin Care and Rejuvenation Procedures. journal of drugs in dermatology 2016; 15(4s): S63-S71, and Widgerow A. TOPICAL SKIN RESTORATION TECHNOLOGY — ADVANCES IN AGE MANAGEMENT STRATEGIES. MODERN AESTHETICS 2016; (May/June): 1-8.

With respect to Anethum graveolens/Dill extract, it produces a reinduction of LOXL synthesis. See Cenizo V, André V, Reymermier C, Sommer P, Damour O, E. P. LOXL as a target to increase the elastin content in adult skin: a dill extract induces the LOXL gene expression. Experimental Dermatology 2006; 15: 574-81. While microfibrils and soluble elastin continue to be synthesized throughout life, LOXL dramatically decreases from the age of 18. Increased levels of LOXL in the skin causes the assembly of microfibrils and tropoelastin, leading to improved mechanical properties of the skin. Elastogenesis mainly occurs until the end of the second decade of the life, although the global content of skin elastin can increase after that, the nature of this elastin protein is often suboptimal and dysfunctional. After this period, the elastin gene and fibrillin-1 gene are still active throughout the life although elastogenesis becomes low or inefficient. Therefore, elastin and fibrillin-1 themselves are not really the missing targets to reinduce elastogenesis but LOXL, which declines after the first decades of life, has been shown to stimulate elastogenesis and maintain elastic fibers homeostasis. See Liu X, Zhao Y, Gao J, et al. Elastic fiber homeostasis requires lysyl oxidase-like 1 protein. Nat Genet 2004; 36(2): 178-82. Dill extract was shown to increase the expression of LOXL in fibroblasts and in the skin engineering models and to affect de novo elastogenesis in vivo.

In some embodiments, formulations as described herein comprise dill extract. In some embodiments, the dill extract is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the dill extract is provided in a range of about 0.25% to about 10%, about 0.025% to about 4%, about 0.5% to about 8%, about 0.75% to about 6%, or about 1% to about 4% by weight. In some embodiments, the dill extract is provided at about 1.0% by weight.

With respect to unroasted shea butter extract and avocado seed extract, they are entrapped in an active multi-lamellar mineral clay (bentonite). In adipocytes, lipolysis naturally occurs to generate energy by hydrolysis of stored triglycerides into fatty acids and glycerol which are then easily released from the cells. See Russell S T, Tisdale M J. Studies on the antiobesity effect of zinc-alpha2-glycoprotein in the ob/ob mouse. Int J Obes (Loud) 2011; 35(3): 345-54. This biochemical reaction is regulated by cAMP which activates the Hormone Sensitive Lipase (HSL), the enzyme involved in the hydrolysis. Shea butter extract increases the cAMP level through a pathway acting on Zinc alpha-2-Glycoprotein (ZAG). ZAG is a protein secreted by both adipocytes and keratinocytes—it stimulates cAMP, leading to improvement in lipolysis with a caffeine-like efficacy.

Elastase is a serine protease involved in the degradation of elastin fibers which accelerates loss of dermis density and firmness. See Alkemade J, Molhuizen H, Ponec M, et al. SKALP/elafin is an inducible proteinase inhibitor in human epidermal keratinocytes. Journal of Cell Science 1994; 107: 2335-42. Avocado seed extract is able to stimulate SKALP (SKin-derived AntiLeukoProteinase), an elastase inhibitor, inhibiting elastase activity and slowing down the dermis degradation providing a firmer skin. Silanols contained in the bentonite are known to regenerate extra cellular matrix (ECM) through increased stimulation of fibroblast growth. Clinical studies have demonstrated that silanols stimulate the production of collagen and elastin fibers leading to remodeling of the dermal fiber architecture and an overall improvement of the skin surface. See Emami-Razavi S, Esmaeili N, Forouzannia S, et al. EFFECT OF BENTONITE ON SKIN WOUND HEALING: EXPERIMENTAL STUDY IN THE RAT MODEL. Acta Medica Iranica 2006; 44(4): 235-40, and Mahmoudi M, Adib-Hajbaghery M, Mashaiekhi M. Comparing the effects of Bentonite & Calendula on the improvement of infantile diaper dermatitis: A randomized controlled trial. The Indian Journal of Medical Research 2015; 142(6): 742-6.

In some embodiments, formulations as described herein comprise Euglena gracilis extract, aqua, caffeine, Glaucium flavum leaf extract, or combinations thereof. Euglena gracilis extract, aqua, caffeine, and Glaucium flavum leaf extract activate lipolysis, promotes unbinding of adipocytes from ECM by stimulating proteases, phosphodiesterases. In some embodiments, these extracts work synergistically to increase lipolysis, stimulate proteases and phosphodiesterase that release adipocytes from the ECM encouraging their breakdown and absorption. See Product monograph: sederma phytosonic Sept 2008. In some embodiments, caffeine improves skin barrier function and improve photodamage and skin texture. See Brandner J, Behne M, B H, Moll I. Caffeine improves barrier function in male skin. International Journal of Cosmetic Science 2006; 28: 343-7 and Koo S W, Hirakawa S, Fujii S, Kawasumi M, Nghiem P. Protection from photodamage by topical application of caffeine after ultraviolet irradiation. Br J Dermatol 2007; 156(5): 957-64.

In some embodiments, formulations as described herein comprising Euglena gracilis extract, aqua, caffeine, and Glaucium flavum leaf extract are provided at least or about 0.001%, 0.002%, 0.005%, 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Euglena gracilis extract, aqua, caffeine, and Glaucium flavum leaf extract are provided in a range of about 0.001% to about 6%, about 0.002% to about 4%, about 0.01% to about 3%, or about 0.02% to about 2% by weight. In some embodiments, the Euglena gracilis extract, aqua, caffeine, and Glaucium flavum leaf extract are provided at about 0.20% by weight.

Increasing GAGs (Glycosaminoglycans) such as Hyaluronic Acid (HA)—Smoothing, Improved Texture and Decreased Crepiness

Hydroxymethoxyphenyl decanone is a potent intrinsic hyaluronic acid booster, antioxidant and anti-irritant. Polyholosides from flax seeds include xylose, galactose, arabinose, rhamnose; Xylose, the main pentose included here is the first essential constituent of GAGs and consequently regulates their synthesis. Phosphatidylserine, a Lipoid, provides MMP1 control, procollagen increase, stimulates HA production. Saccharomyces cerevisiae is a stressed cellular protoplasm yeast extract that improves fibroblast cellular oxygenation and formation of procollagen and stimulates intrinsic HA production.

With respect to hydroxymethoxyphenyl decanone, it is a potent hyaluronic acid booster, antioxidant and anti-irritant. It has been demonstrated to stimulate the dermal AND epidermal hyaluronic acid level by 259% and 198% versus placebo, respectively in ex vivo human skin model. See Product monograph: Symdecanox, Symrise June 2015.

In some embodiments, formulations as described herein comprise hydroxymethoxyphenyl decanone. In some embodiments, the hydroxymethoxyphenyl decanone is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the hydroxymethoxyphenyl decanone is provided in a range of about 0.25% to about 10%, about 0.5% to about 8%, about 0.75% to about 6%, about 1% to about 4%,or about 0.5% to about 2% by weight. In some embodiments, the hydroxymethoxyphenyl decanone is provided at about 1.0% by weight.

With respect to polyholosides from flax seeds/linseed, they stimulate glycosaminoglycan (GAG) synthesis. GAGs are fundamental components of the dermis comprising long unbranched chains of high molecular weight consisting of repeating saccharide units. The GAGs synthesis is initiated by the sequential addition of four monosaccharides: xylose-galactose-galactose-glucuronic acid. Xylose, the main pentose of the polyholoside, is the first essential constituent of GAGs and consequently regulates their synthesis. See Wen J, Xiao J, Randar M, et al. Xylose phosphorylation functions as a molecular switch to regulate proteoglycan biosynthesis. Proc Natl Acad Sci USA 2014; 111(44): 15723-8.

In some embodiments, formulations as described herein comprise polyholosides. In some embodiments, the polyholosides are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the polyholosides are provided in a range of about 0.25% to about 10%, about 0.5% to about 8%, about 0.75% to about 6%, about 1% to about 4%, or about 2.5% to about 10% by weight. In some embodiments, the polyholosides are provided at about 5.0% by weight.

With respect to phosphatidylserine (PS), aside from its ability to decrease MMP-1 and increase procollagen, it also stimulates intrinsic production of HA. See Cho S, Kim H H, Lee M J, et al. Phosphatidylserine prevents UV-induced decrease of type I procollagen and increase of MMP-1 in dermal fibroblasts and human skin in vivo. J Lipid Res 2008; 49(6): 1235-45, and Lee S-H, Yang J-H, Park Y-K, et al. Protective effect and mechanism of phosphatidylserine in UVB-induced human dermal fibroblasts. European Journal of Lipid Science and Technology 2013; 115(7): 783-90 In-vitro data on human fibroblast cells shows that PS up-regulates the expression of hyaluronan synthase II enzyme (also called HAS2). This enzyme is a key enzyme for the production of hyaluronic acid within the skin cells. Additional data on artificial skin confirm the up-regulation of hyaluronic acid formation in the presence of PS. See Product monograph; Nagase Chemtex PIPS; Phosphatidylserine & phosphatidylinositol; May 2015.

In some embodiments, formulations as described herein comprise phosphatidylserine. In some embodiments, the phosphatidylserine is provided at least or about 0.001%, 0.002%, 0.005%, 0.01%, 0.02%, 0.03%, 0.04%, 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the phosphatidylserine is provided in a range of about 0.001% to about 6%, about 0.002% to about 4%, about 0.01% to about 3%, about 0.02% to about 2%, or about 0.25% to about 1% by weight. In some embodiments, the phosphatidylserine is provided at about 0.05% by weight. In some embodiments, the phosphatidylserine is provided at about 0.25% by weight. In some embodiments, the phosphatidylserine is provided at about 1% by weight.

With respect to Saccharomyces cerevisiae, it increases cellular oxygenation and wound healing while promoting collagen, elastin and HA synthesis. In addition the extract I has been used effectively for reduction in erythema and reduction in sunburn pain. See Product monologue: Active Concepts 2014.

In some embodiments, Euglena gracilis extract, aqua, caffeine, Glaucium flavum leaf extract, or combinations thereof are used to increase glycosaminoglycans (GAGs). For example, Euglena gracilis extract, aqua, caffeine, Glaucium flavum leaf extract, or combinations thereof increase hyaluronic acid (HA).

In some embodiments, formulations as described herein comprise Tremella fuciformis extract or Tremella. Tremella fuciformis extract is derived from an edible mushroom. In some embodiments, Tremella fuciformis extract provides moisture and serve as a natural hyaluronic acid. In some embodiments, Tremella fuciformis extract provides anti-oxidant properties. In some embodiments, Tremella fuciformis extract or Tremella is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, Tremella fuciformis extract or Tremella is provided in a range of about 0.25% to about 10%, about 0.5% to about 8%, about 0.75% to about 6%, about 0.5% to about 2.0%, or about 1% to about 4% by weight. In some embodiments, Tremella fuciformis extract or Tremella is provided at about 0.5%. In some embodiments, Tremella fuciformis extract or Tremella is provided at about 1.0%. In some embodiments, Tremella fuciformis extract or Tremella is provided at about 2.0%.

Soothing, Softening Scar Tissue, Smoothing, Pain Relieving, AOX/Pain Relief/Scar Tissue

Saccharomyces cerevisiae is a stressed cellular protoplasm yeast extract, it provides a soothing calming effect on sunburned and tender skin and softening of underlying scar tissue. Phytoene/Phytofluene, or Colorless Carotenoids, exhibit anti-oxidative, anti-inflammatory, skin brightening, and UV absorbency properties. Centella asiatica hastens healing, stimulates collagen, fibronectin, prevents scarring.

With respect to Saccharomyces cerevisiae, it increases cellular oxygenation and wound healing while promoting collagen, elastin and HA synthesis. In addition, the extract I has been used effectively for reduction in erythema and reduction in sunburn pain. See Product monologue: Active Concepts 2014.

With respect to phytoene/phytofluene, they are natural colorless carotenoids derived from saltwater micro-algae and used by them for protection against UV radiation and environmental stress. They exhibit anti-oxidant and anti-inflammatory effects (inhibit PGE-2, pro-inflammatory cytokines IL-6 and IL-1 and reduce MMP-1 production).

In some embodiments, formulations as described herein comprise phytoene/phytofluene. In some embodiments, the phytoene/phytofluene is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the phytoene/phytofluene is provided in a range of about 0.25% to about 10%, about 0.5% to about 8%, about 0.75% to about 6%, about 1% to about 4%, or about 0.2% to about 1% by weight. In some embodiments, the phytoene/phytofluene is provided at about 0.2% by weight. In some embodiments, the phytoene/phytofluene is provided at about 0.5% by weight. In some embodiments, the phytoene/phytofluene is provided at about 1.0% by weight.

With respect to Centella asiatica, it is effective in improving treatment of small wounds, hypertrophic wounds as well as burns, psoriasis and scleroderma. The mechanism of action involves promoting fibroblast proliferation and increasing the synthesis of collagen and intracellular fibronectin content and also improvement of the tensile strength of newly formed skin as well as inhibiting the inflammatory phase of hypertrophic scars and keloids. Research results indicate that it can be used in the treatment of photoaging skin, cellulite and striae. Bylka W, Znajdek-Awizen P, Studzinska-Sroka E, Brzezinska M. Centella asiatica in cosmetology. Postepy Dermatol Alergol 2013; 30(1): 46-9

In some embodiments, formulations as described herein comprise Centella asiatica. In some embodiments, the Centella asiatica is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the Centella asiatica is provided in a range of about 0.25% to about 10%, about 0.5% to about 8%, about 0.75% to about 6%, or about 1% to about 4% by weight. In some embodiments, the Centella asiatica is provided at about 1.0% by weight.

Reverse Cellular Senescence—Wake Up Sleeping Dormant Fibroblasts to Produce New Collagen and Elastin and Builds ECM—Improving Tone and Texture of Skin

Described herein, in some embodiments, are formulations for reversing cellular senescence. In some embodiments, the formulations reverse fibroblast senescence. In some embodiments, the formulation stimulates collagen and elastin formation. In some embodiments, formulations as described herein comprise Plantago lanceolata. In some embodiments, formulations as described herein comprise oleuropein.

Anti-Inflammatory, Pigmentary Control—Improve Pigmentation Problems Particularly Decollete—AOX/Pigmentation

Described herein, in some embodiments, are formulations for pigmentary control. In some embodiments, formulations for pigmentary control improve redness. In some embodiments, formulations for pigmentary control comprise phytoene/phytofluene. In some embodiments, formulations for pigmentary control comprise niacinamide.

Niacinamide or nicotinamide is a biologically active form of niacin (vitamin B3) is well tolerated by the skin. It has been used to treat can and demonstrated to increase ceramide and skin cholesterol levels. In addition, it has been found effective in reducing cutaneous pigmentation by the suppression of melanosome transfer from melanocytes to keratinocytes. See HAKOZAKI T, MINWALLA L, ZHUANG J, et al. The effect of niacinamide on reducing cutaneous pigmentation and suppression of melanosome transfer. British Journal of Dermatology 2002; 147: 20-31 and Navarrete-Solis J, Castanedo-Cazares J P, Tones-Alvarez B, et al. A Double-Blind, Randomized Clinical Trial of Niacinamide 4% versus Hydroquinone 4% in the Treatment of Melasma. Dermatol Res Pract 2011; 2011: 379173. Niacinamide comprises barrier-protective, anti-inflammatory and depigmenting effects. See Wohlrab J, Kreft D. Niacinamide-mechanisms of action and its topical use in dermatology. Skin Pharmacol Physiol 2014; 27(6): 311-5.

In some embodiments, niacinamide is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, niacinamide is provided in a range of about 0.25% to about 10%, about 0.5% to about 8%, about 0.75% to about 6%, or about 1% to about 4% by weight. In some embodiments, niacinamide is provided at about 1% by weight. In some embodiments, niacinamide is provided at about 2% by weight. In some embodiments, niacinamide is provided at about 4% by weight.

Improved Barrier Function—Protection Against Water Loss and the Prevention of Substances and Bacteria Penetrating into the Body, Plumps Skin by Improved Hydration

Formulations as described herein, in some embodiments, improve skin barrier function. In some embodiments, formulations for improving skin barrier function comprise niacinamide. In some embodiments, formulations for improving skin barrier function comprise Hydroceramide and hydrogenated lecithin.

In some embodiments, hydrogenated lecithin is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, hydrogenated lecithin is provided in a range of about 0.25% to about 10%, about 0.5% to about 8%, about 0.75% to about 6%, or about 1% to about 4% by weight. In some embodiments, hydrogenated lecithin is provided with C12-16 alcohols, palmitic acid, or combinations thereof. In some embodiments, C12-16 alcohols are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, C12-16 alcohols are provided in a range of about 0.25% to about 10%, about 0.5% to about 8%, about 0.75% to about 6%, or about 1% to about 4% by weight. In some embodiments, palmitic acid is provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, palmitic acid is provided in a range of about 0.25% to about 10%, about 0.5% to about 8%, about 0.75% to about 6%, or about 1% to about 4% by weight. In some embodiments, hydrogenated lecithin, C12-16 alcohols, and palmitic acid are provided at least or about 0.05%, 0.10%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, hydrogenated lecithin, C12-16 alcohols, and palmitic acid are provided in a range of about 0.25% to about 10%, about 0.5% to about 8%, about 0.75% to about 6%, about 1% to about 4%, or about 1% to about 6% by weight. In some embodiments, hydrogenated lecithin, C12-16 alcohols, and palmitic acid are provided at about 4% by weight. In some embodiments, hydrogenated lecithin, C12-16 alcohols, and palmitic acid are provided at about 5% by weight.

The ‘skin barrier’ functions as a natural frontier between the inner organism and the environment. It is comprised mainly by the epidermis and provides a physical (lipids, corneocytes and an acidic film on the skin surface) and a biochemical barrier provided by the slightly acidic pH. This provides for cutaneous antimicrobial defense and regulates epidermal enzyme activity and expression. The interaction of transepidermal water loss (TEWL), stratum corneum hydration (SC hydration), sebum level on the skin and the skin surface pH value maintains skin barrier functionality and skin appearance. High levels of TEWL correlate with high pH, low stratum corneum hydration and reduced skin surface lipid. There seem to be differences depending on the body site, as TEWL increases significantly with ageing at the décolleté, whereas it decreases significantly at forehead and cheek. See Luebberding S, Krueger N, Kerscher M. Age-related changes in skin barrier function - quantitative evaluation of 150 female subjects. Int J Cosmet Sci 2013; 35(2): 183-90. Hydroceramide can reinforce the natural lipid barrier of dry and aging skin and also shows an ability to maintain the moisture balance of skin. In addition hydrogenated lecithin is a natural phospholipid based emulsifier that efficiently penetrates the stratum corneum while preserving skin integrity by merging with the skin and forming a second barrier layer and providing excellent hydration to the skin surface layers.

Other Activity

Caffeine, in vectorized form (with sodium salicylate and lecithin), can also be included in the formulation to promote lipolysis. In some embodiments, the caffeine is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the caffeine is provided in a range of about 0.001% to about 6%, about 0.002% to about 4%, about 0.01% to about 3%, or about 0.02% to about 2%. In some embodiments, caffeine is provided with sodium salicylate, lecithin, silica, or combinations thereof. In some embodiments, the sodium salicylate, lecithin, or silica are each provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the sodium salicylate, lecithin, or silica are each provided in a range of about 0.001% to about 6%, about 0.002% to about 4%, about 0.01% to about 3%, or about 0.02% to about 2% by weight. In some embodiments, the caffeine, sodium salicylate, lecithin, and silica are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the caffeine, sodium salicylate, lecithin, and silica are provided in a range of about 0.001% to about 6%, about 0.002% to about 4%, about 0.01% to about 3%, or about 0.02% to about 2% by weight. In some embodiments, the caffeine, sodium salicylate, lecithin, and silica are provided at about 0.02% by weight.

Formulations as described herein, in some embodiments, comprise ceramide NP. In some embodiments, the ceramide NP is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the ceramide NP is provided in a range of about 0.001% to about 6%, about 0.002% to about 4%, about 0.01% to about 3%, about 0.02% to about 2%, or about 0.50% to about 0.20% by weight. In some embodiments, the ceramide NP is provided at about 0.05% by weight. In some embodiments, the ceramide NP is provided at about 0.10% by weight. In some embodiments, the ceramide NP is provided at about 0.20% by weight.

Methods of Use

The compositions described herein are useful in conjunction with various skin procedures or surgical procedures. In some embodiments, the compositions are topical compositions. Topical compositions described herein can modulate inflammation and wound healing. In some embodiments, the topical compositions described herein accelerate the healing process, accelerate clearance of products including lipid particles, induce inflammatory and regenerative gene expression, accelerate resolution of inflammation stimulates extracellular matrix remodeling, reduce induration, reduce fibrous banding, reduce pain or discomfort, or combinations thereof. In some embodiments, the topical compositions described herein improve macrophage clearance, autophagy (e.g., lipophagy), ECM remodeling, or combinations thereof. In some embodiments, the topical compositions described herein modulate various inflammatory mediators and markers. In some embodiments, the topical compositions described herein modulate a wound micro-environment. In some embodiments, the topical compositions described herein modulate a wound micro-environment to improve the healing process. In some embodiments, the topical compositions described herein stimulate the proliferation of keratinocytes, fibroblasts, and endothelial cells, encouraging angiogenesis and the synthesis of ECM molecules to restore damaged tissue.

Described herein are compositions to modulate inflammation, improve wound healing, improve skin laxity, improve body contouring, or combinations thereof. In some embodiments, the formulations improve skin laxity of body contouring. In some embodiments, the topical compositions are administered in an amount sufficient to modulate an expression level of one of more inflammatory or regenerative genes. In certain aspects, the topical compositions are applied to a skin region of the subject in an amount sufficient to modulate an expression level of one or more inflammatory or regenerative genes, wherein the topical compositions are administered before a surgical procedure, after a surgical procedure, or both. In certain aspects, the topical compositions are applied to a skin region of the subject before surgical procedure and after surgical procedure in an amount sufficient to modulate an expression level of one or more inflammatory or regenerative genes. In certain aspects, the topical compositions are applied to a skin region of the subject in an amount sufficient to modulate an expression level of one or more inflammatory or regenerative genes, wherein the topical compositions are administered before a procedure to remove fat and skin, after a procedure to remove fat and skin, or both.

In some embodiments, the administration of the topical compositions modulate one or more inflammatory or regenerative genes. In some embodiments, the administration of the topical compositions modulate inflammatory or regenerative genes involved in a pro-inflammatory response. In some embodiments, the administration of the topical compositions modulate inflammatory or regenerative genes involved in an anti-inflammatory response and M2 macrophage activation profile. In some embodiments, the administration of the topical compositions modulate inflammatory or regenerative genes involved in autophagy. In some embodiments, the administration of the topical compositions modulate inflammatory or regenerative genes involved in M1 macrophage stimulation. In some embodiments, the administration of the topical compositions modulate inflammatory or regenerative genes involved in an anti-inflammatory response. In some embodiments, the administration of the topical compositions modulate inflammatory or regenerative genes involved in extracellular matrix remodeling.

In some embodiments, the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof. In some embodiments, the one or more inflammatory or regenerative genes encodes a chemokine receptor, a chemokine ligand, an angiotensin receptor, a complement component, a cholinergic receptor, a clusterin, a cytochrome, a fibroblast growth factor, a hepatocyte growth factor, an interleukin receptor, an interleukin, an integrin, a killer cell like lectin receptor, a leukotriene synthase, a lymphocyte antigen, a nuclear factor of activated T-cell, a Nod-like receptor (NLR), a phospholipase, a serpin, a single Ig IL-1-related receptor, a sialic acid binding Ig like lectin, a tachykinin receptor, a TNF receptor, a tyrosylprotein sulfotransferase, or a fragment or variant thereof. In some embodiments, the one or more inflammatory or regenerative genes encodes a chemokine-like receptor, a chemokine ligand, a cluster of differentiation ligand, a cholinergic receptor, a fms related tyrosine kinase ligand, a growth differentiation factor, an interleukin, an interleukin receptor, a lymphocyte antigen, a matrix metallopeptidase, a Nod-like receptor (NLR), a phospholipase, a signal peptide, a CUB domain and EGF like domain, a sialophorin, a transglutaminase, or a fragment or variant thereof. In some embodiments, the one or more inflammatory or regenerative genes comprises AGTR1, C1R, C3, CCL20, CCRL2, CLU, CYBB, FIGF, IL21R, ITGB2, KLRG1, LTC4S, LY86, NFAM1, NFATC4, NLRP3, PLCB2, SERPINA1, SIGLEC1, or TNFSF13B. In some embodiments, the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In some embodiments, the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In some embodiments, the one or more inflammatory or regenerative genes comprises ILI, IL6, or TNFA. In some embodiments, the one or more inflammatory or regenerative genes comprises IL6. In some embodiments, the one or more inflammatory or regenerative genes comprises C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In some embodiments, the one or more inflammatory or regenerative genes comprises C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. In some embodiments, the one or more inflammatory or regenerative genes comprises ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In some embodiments, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In some embodiments, the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. In some embodiments, the one or more inflammatory or regenerative genes comprises C1R or SCUBE1. In some embodiments, the one or more inflammatory or regenerative genes comprises TGM2 or PLCB2.

In some embodiments, the administration of the topical composition modulates two or more inflammatory or regenerative genes. In some embodiments, the two or more inflammatory or regenerative genes comprise ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. In some embodiments, the two or more inflammatory or regenerative genes comprise ILI, IL6, or TNFA In some embodiments, the two or more inflammatory or regenerative genes comprise AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. In some embodiments, the two or more inflammatory or regenerative genes comprise C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. In some embodiments, the two or more inflammatory or regenerative genes comprise C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2.

In some embodiments, the administration of the topical composition increases the expression of one or more inflammatory or regenerative genes by at least 1.25 fold, at least 1.5 fold, at least 1.75 fold, at least 2 fold, at least 2.5 fold, at least 3 fold, at least 4 fold, or at least 5 fold as compared to control. In some embodiments, the expression level is increased by at least 1.25-fold as compared to control. In some embodiments, the expression level is increased by at least 1.5-fold as compared to control. In some embodiments, the expression level is increased by at least 2-fold as compared to control. In some embodiments, the expression level is increased by at least 3-fold as compared to control.

In some embodiments, the control is a skin region that does not receive the topical composition described herein. In some embodiments, the control is a skin region that receives a bland moisturizer or comparator product. In some embodiments, the bland moisturizer or comparator product does not comprise one or more peptides. In some embodiments, the control is a baseline expression level.

In some embodiments, the administration of the topical composition increases the expression of one or more inflammatory or regenerative genes after at least about 1 day, at least about 2 days, at least about 3 days, at least about 4 days, at least about 5 days, or at least about 6 days. In some embodiments, the expression level is increased after at least about 1 week, at least about 2 weeks, at least about 3 weeks, at least about 4 weeks, at least about 5 weeks, at least about 6 weeks, at least about 7 weeks, at least about 8 weeks, at least about 9 weeks, or at least about 10 weeks. In some embodiments, the expression level is increased after at least about 2 weeks. In some embodiments, the expression level is increased after at least about 4 weeks. In some embodiments, the expression level is increased from 1 to 6 weeks after administration. In some embodiments, the expression level is increased from 1 to 5 weeks after administration. In some embodiments, the expression level is increased from 1 to 4 weeks after administration. In some embodiments, the expression level is increased from 1 to 3 weeks after administration. In some embodiments, the expression level is increased from 1 to 2 weeks after administration. In some embodiments, the expression level is increased from 2 to 6 weeks after administration. In some embodiments, the expression level is increased from 2 to 5 weeks after administration. In some embodiments, the expression level is increased from 2 to 4 weeks after administration. In some embodiments, the expression level is increased from 2 to 3 weeks after administration.

In some embodiments, the methods further comprise, subsequent to administration of the topical compositions, detecting the expression level of the at least one gene by contacting a sample obtained from the treated skin region of the subject with a probe that recognizes the at least one gene and detect binding between the at least one gene and the probe. In some embodiments, the methods further comprise, before administration of the topical compositions, detecting expression level of the one or more inflammatory or regenerative genes comprising ACKR4, AGTR1, C1R, C1S C3, CCL17, CCL19, CCL20, CCL22, CCRL1, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B by contacting a skin sample of the subject with a probe that recognizes the one or more inflammatory or regenerative genes comprising ACKR4, AGTR1, C1R, C1S C3, CCL17, CCL19, CCL20, CCL22, CCR19, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B and detect binding between the gene and the probe.

The topical compositions described herein, in some embodiments, are administered daily, every day, every alternate day, five days a week, once a week, every other week, two weeks per month, three weeks per month, once a month, twice a month, three times per month, or more. In some embodiments, the topical compositions described herein are administered twice daily, e.g., morning and evening. In some embodiments, the topical compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, the topical compositions described herein are administered twice daily for at least or about 1 week, 2 weeks, 3 weeks, 1 month, 2 months, 3 months, 4 months, 5 months, 6 months, or more. In some embodiments, the topical compositions described herein are administered once daily, twice daily, three times daily, four times daily, five times daily, six times daily, or more than six times daily for at least or about 1 week, 2 weeks, 3 weeks, 1 month, 2 months, 3 months, 4 months, 5 months, 6 months, or more.

In some embodiments, the topical compositions described herein are used in conjunction with a skin procedure. In some embodiments, the skin procedure comprises a laser treatment, a chemical peel, microdermabrasion, microneedling, or radiofrequency microneedling.

In some embodiments, the topical compositions described herein are used in conjunction with a procedure including, but not limited to, high frequency focused ultrasound, pulsed focus ultrasound, cryolipolysis, radiofrequency induced electroporation. In some embodiments, the procedure comprises low level laser therapy, infrared light, ultrasound, radiofrequency, or cryolipolysis. In some instances, the procedure comprises an energy source. In some instances, the energy source is electromagnetic energy. In some instances, the procedure is high intensity focused electro-magnetic technology (HIFEM).

In some embodiments, the topical compositions described herein are used in conjunction with a surgical procedure. In some embodiments, the surgical procedure is a surgical skin removal procedure. In some embodiments, the surgical procedure comprises a body shaping procedure. In some embodiments, the body shaping procedure comprises the injection of a filler or lipolytic agent. In some embodiments, the surgical procedure comprises the injection of submental deoxycholic acid (DCA). In some embodiments, the surgical procedure comprises a panniculectomy (removal of excess skin in the lower abdominal region), an abdominoplasty (tummy tuck), a liposuction, or an excisional body lift. An excisional body lift may include a lower body lift, an arm lift (brachioplasty),an inner thigh lift, a buttock augmentation, a circumferential body lift (belt procedure), a breast lift, a breast reduction, a breast augmentation, or a labiaplasty.

In some embodiments, the volume of DCA administered is at least about 0.1 cc, at least about 0.2 cc, at least about 0.3 cc, at least about 0.4 cc, at least about 0.5 cc, at least about 0.6 cc, at least about 0.7 cc, at least about 0.8 cc, at least about 0.9 cc, or at least about 1.0 cc. In some embodiments, the volume of DCA administered is at least about 1 cc, at least about 2 cc, at least about 3 cc, at least about 4 cc, at least about 5 cc, at least about 6 cc, at least about 7 cc, at least about 8 cc, at least about 9 cc, at least about 10 cc, at least about 11 cc, or at least about 12 cc. In some embodiments, the volume of DCA administered is at least about 8 cc.

In some instances, the topical compositions described herein are administered before a skin procedure or surgical procedure. In additional instances, the topical compositions described herein are administered as a pre-conditioning treatment. In some instances, the topical composition described herein are administered for at least 1 day, 2 days, 3 days, 4 days, 5 days, 6 days, 1 week, 2 weeks, 3 weeks, 1 month, 2 months, 3 months, 4 months, 5 months, 6 months, or more as a pre-conditioning treatment. In some instances, the topical compositions described herein are administered for at least 2-8 weeks, 2-6 weeks, 2-4 weeks, or 2-3 weeks as a pre-conditioning treatment. In some cases, the topical compositions described herein are administered at least 1 day, 2 days, 3 days, 4 days, 5 days, 6 days, 1 week, 2 weeks, 3 weeks, 1 month, 2 months, 3 months, 4 months, 5 months, 6 months, or more before a skin procedure or surgical procedure. In some instances, the topical compositions described herein are administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours after a skin procedure or surgical procedure. In some cases, the topical compositions described herein are administered at least or up to 1 day, 2 days, 3 days, 4 days, 5 days, 6 days, 1 week, 2 weeks, 3 weeks, 1 month, 2 months, 3 months, 4 months, 5 months, 6 months, or more after a skin procedure or surgical procedure. In some embodiments, the topical compositions described herein are administered for at least 2-8 weeks, 2-6 weeks, 2-4 weeks, or 2-3 weeks after a skin procedure or surgical procedure. Sometimes the topical compositions described herein are administered singly, or over a time course, such as daily, multiple times weekly, weekly, biweekly, monthly or less frequently before or after a skin procedure or surgical procedure. In some instances, the topical compositions described herein are administered singly, or over a time course, such as daily, multiple times weekly, weekly, biweekly, monthly or more frequently before or after a skin procedure or surgical procedure. In some instances, the topical compositions described herein are administered once per day, twice per day, three times per day or more after the end of skin procedure or surgical procedure. In some instances, the topical compositions described herein are administered twice daily administration, e.g., morning and evening, after the end of a skin procedure or surgical procedure. In some embodiments, the topical composition is administered for at least 2 weeks before the skin procedure or surgical procedure. In some embodiments, the topical compositions described herein are administered once daily, twice daily, three times daily, four times daily, five times daily, six times daily, or more than six times daily for at least for at least about 1 week, 2 weeks, 3 weeks, 4 weeks, 5 weeks, or more than 5 weeks before the skin procedure or surgical procedure. In some embodiments, the topical compositions described herein are administered once daily, twice daily, three times daily, four times daily, five times daily, six times daily, or more than six times daily for at least for at least about 2 weeks before the skin procedure or surgical procedure.

In some embodiments, the topical compositions are administered after the skin procedure or surgical procedure. In some embodiments, the topical compositions are administered for at least 1 day, 2 days, 3 days, 4, days, 5, days, 6, days, or 7 days after the skin procedure or surgical procedure. In some instances, the topical compositions described herein are administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours after a skin procedure or surgical procedure. Sometimes the topical compositions described herein are administered singly, or over a time course, such as daily, multiple times weekly, weekly, biweekly, monthly or less frequently after a skin procedure or surgical procedure. In some instances, the topical compositions described herein are administered singly, or over a time course, such as daily, multiple times weekly, weekly, biweekly, monthly or more frequently after a skin procedure or surgical procedure. In some embodiments, the topical compositions are topical compositions. In some instances, the topical compositions are administered twice daily for at least or about 1 week, 2 weeks, 3 weeks, 1 month, 2 months, 3 months, 4 months, 5 months, 6 months, or more after a skin procedure or surgical procedure. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more after a skin procedure or surgical procedure. In some embodiments, the topical compositions described herein are administered once daily, twice daily, three times daily, four times daily, five times daily, six times daily, or more than six times daily for at least for at least about 1 week, 2 weeks, 3 weeks, 4 weeks, 5 weeks, or more than 5 weeks after a skin procedure or surgical procedure. In some embodiments, the topical composition is administered for at least 2 weeks after a skin procedure or surgical procedure. In some embodiments, the topical compositions described herein are administered once daily, twice daily, three times daily, four times daily, five times daily, six times daily, or more than six times daily for at least for at least about 2 weeks after the skin procedure or surgical procedure.

In some instances, the topical compositions described herein are administered before a skin procedure or surgical procedure and after a skin procedure or surgical procedure. In additional instances, the topical compositions described herein are administered as a pre-conditioning treatment. In some instances, the topical composition described herein are administered for at least 1 day, 2 days, 3 days, 4 days, 5 days, 6 days, 1 week, 2 weeks, 3 weeks, 1 month, 2 months, 3 months, 4 months, 5 months, 6 months, or more as a pre-conditioning treatment and administered at least 1 day, 2 days, 3 days, 4 days, 5 days, 6 days, 1 week, 2 weeks, 3 weeks, 1 month, 2 months, 3 months, 4 months, 5 months, 6 months, or more after a skin procedure or surgical procedure. In some instances, the topical compositions described herein are administered for at least 2-8 weeks, 2-6 weeks, 2-4 weeks, or 2-3 weeks as a pre-conditioning treatment and administered at least 2-8 weeks, 2-6 weeks, 2-4 weeks, or 2-3 weeks after a skin procedure or surgical procedure. Sometimes the topical compositions described herein are administered singly, or over a time course, such as daily, multiple times weekly, weekly, biweekly, monthly or less frequently before or after a procedure to reduce skin laxity. In some instances, the topical compositions described herein are administered singly, or over a time course, such as daily, multiple times weekly, weekly, biweekly, monthly or more frequently before and after a skin procedure or surgical procedure to reduce skin laxity. In some instances, the topical compositions described herein are administered once per day, twice per day, three times per day or more before and after a skin procedure or surgical procedure. In some instances, the topical compositions described herein are administered twice daily administration, e.g., morning and evening, before and after a skin procedure or surgical procedure.

In some instances, compositions as described herein are applied to a submental region, abdomen, face, flank, back, chest, arm, leg, buttock, or combination thereof before a skin procedure or surgical procedure. In some instances, compositions as described herein are applied to a submental region, abdomen, face, flank, back, chest, arm, leg, buttock, or combination thereof after a skin procedure or surgical procedure. In some instances, compositions as described herein are applied to a submental region, abdomen, face, flank, back, chest, arm, leg, buttock, or combination thereof before a skin procedure and after a skin procedure or surgical procedure. In some instances, compositions as described herein accelerate the healing process, accelerate clearance of products including lipid particles, induce inflammatory and regenerative gene expression, accelerate resolution of inflammation stimulates extracellular matrix remodeling, reduce induration, reduce fibrous banding, reduce pain or discomfort, or combinations thereof when applied to a submental region, abdomen, face, flank, back, chest, arm, leg, buttock, or combination thereof before a skin procedure or surgical procedure. In some instances, compositions as described herein accelerate the healing process, accelerate clearance of products including lipid particles, induce inflammatory and regenerative gene expression, accelerate resolution of inflammation stimulates extracellular matrix remodeling, reduce induration, reduce fibrous banding, reduce pain or discomfort, or combinations thereof when applied to a submental region, abdomen, face, flank, back, chest, arm, leg, buttock, or combination thereof after a skin procedure or surgical procedure. In some instances, compositions as described herein accelerate the healing process, accelerate clearance of products including lipid particles, induce inflammatory and regenerative gene expression, accelerate resolution of inflammation stimulates extracellular matrix remodeling, reduce induration, reduce fibrous banding, reduce pain or discomfort, or combinations thereof when applied to a submental region, abdomen, face, flank, back, chest, arm, leg, buttock, or combination thereof before a skin procedure or surgical procedure and after a skin procedure or surgical procedure.

In some instances, compositions as described herein reduce induration, edema, skin discoloration, ecchymosis, subcutaneous banding, pain, or combinations thereof. In some instances, compositions as described herein induration, edema, skin discoloration, ecchymosis, subcutaneous banding, pain, or combinations thereof by about 10%, about 20%, about 30%, about 40%, about 50%, about 60%, about 70%, about 80%, about 90%, about 95%, or more than about 95%.

In some embodiments, composition as described herein leads to a reduction in induration, edema, skin discoloration, ecchymosis, subcutaneous banding, pain, or combinations thereof post-procedure. In some embodiments, composition as described herein leads to a reduction in induration, edema, skin discoloration, ecchymosis, subcutaneous banding, pain, or combinations thereof post-procedure. In some embodiments, composition as described herein leads to about 10%, about 20%, about 30%, about 40%, about 50%, about 60%, or about 70% reduction in induration, edema, skin discoloration, ecchymosis, subcutaneous banding, pain, or combinations thereof post-procedure. In some embodiments, composition as described herein leads to about a 40% reduction in induration, edema, skin discoloration, ecchymosis, subcutaneous banding, pain, or combinations thereof post-procedure. In some embodiments, composition as described herein leads to a reduction in induration, edema, skin discoloration, ecchymosis, subcutaneous banding, pain, or combinations thereof post-procedure. In some embodiments, composition as described herein leads to about a 40%, about a 50%, about a 60%, about a 70%, about an 80%, about a 90%, or about a 95% reduction in induration, edema, skin discoloration, ecchymosis, subcutaneous banding, pain, or combinations thereof post-procedure. In some embodiments, the reduction is at least or about 1 day, 2 days, 3 days, 4 days, 5 days, 6 days, 1 week, 2 weeks, 3 weeks, 1 month, 2 months, 3 months, or more than 3 months post-procedure. In some embodiments, the procedure is a fat reduction procedure (e.g., liposuction).

In some instances, composition as described herein when applied results in a reduction in inflammation. In some instances the reduction in inflammation occurs at least or about 1 day, 2 days, 3 days, 4 days, 5 days, 6 days, 1 week, 2 weeks, 3 weeks, 1 month, 2 months, 3 months, 4 months, 5 months, 6 months, or more following a skin procedure or surgical procedure. In some instances, the reduction in inflammation occurs when the topical compositions are administered once daily, twice daily, three times daily, four times daily, or more than four times daily for at least or about 1 week, 2 weeks, 3 weeks, 1 month, 2 months, 3 months, 4 months, 5 months, 6 months, or more following a skin procedure or surgical procedure.

In some instances, composition as described herein when applied results in an improvement in wound healing. In some instances the improvement in wound healing occurs at least or about 1 day, 2 days, 3 days, 4 days, 5 days, 6 days, 1 week, 2 weeks, 3 weeks, 1 month, 2 months, 3 months, 4 months, 5 months, 6 months, or more following a body contouring procedure. In some instances, the improvement in wound healing occurs when the topical compositions are administered once daily, twice daily, three times daily, four times daily, or more than four times daily for at least or about 1 day, 2 days, 3 days, 4 days, 5 days, 6 days, 1 week, 2 weeks, 3 weeks, 1 month, 2 months, 3 months, 4 months, 5 months, 6 months, or more following a body contouring procedure.

In making an assessment, the effects of the topical compositions may be compared to a control. In some instances, an individual or subject topically administers compositions as described herein on one part of the body, and the control comprises a control composition administered to a second part of the body.

### Carrier Systems

Liquids and gels containing the peptides and other components as described herein can be prepared using techniques as are known in the art of cosmetics manufacture. See, e.g., Handbook of Cosmetic Science and Technology, Fourth Edition, edited by André O. Barel, Marc Paye, Howard I. Maibach, CRC Press, 2014, the contents of which is hereby incorporated by reference in its entirety. Various formulations are possible. As an example, a clear cosmetic gel stick composition can include 60 to about 90% of an aliphatic polyhydric alcohol (e.g., a C2-6 alcohol containing from 2 to 6 hydroxyl groups); 1-10% of a soap; and 1-10% of a water-soluble emollient, e.g., a polyoxyalkylene ether of a C8-22 fatty alcohol, as the main ingredients, in combination with the peptides of the preferred embodiments. Aqueous extrudable gels are based on water-oil emulsion technologies. To minimize the amount of water introduced into an extrudable gel formula, the concentration of the active solution is adjusted. Ideally, a high concentration active solution (45-50%) of the peptides can be employed. Carrier systems for AP solids are typically based on volatile cyclic siloxanes because they evaporate quickly and do not leave residue on the skin. As an alternative to volatile cyclic siloxanes, alternatives can be used, including isohexadecane or C13-15 isoalkane. Solidification systems are employed to develop solid sticks that do not melt under typical storage or consumer conditions but provide an elegant skin feel and allow for easy transfer. A combination of cyclopentasiloxane and stearyl alcohol with varying degrees of additional waxes such as hydrogenated castor wax, hydrogenated vegetable oils and polyethylene, can be employed.

For liquid formulations (e.g., gel or lotion forms), a silicone, e.g., a cyclosiloxane or linear silicone (e.g., silicone elastomer), can be employed as a carrier. One type of suitable carrier is a dimethicone crosspolymer gel, e.g., dimethicone crosspolymer in cyclopentasiloxane. Other suitable dimethicone crosspolymers include cyclopentasiloxane, dimethicone/vinyldimethicone crosspolymer; dimethicone, dimethicone/vinyl dimethicone crosspolymer; and isodecane dimethicone/vinyl dimethicone crosspolymer.

Typically, the carrier is present in an amount of from about 80 wt. % to about 95 wt. %, or 82 wt. % to 92 wt. %, e.g., in a topical formulation for application to skin or mucous membranes.

### Other Components

Penetration Enhancers

Fatty acids and alcohols can be employed to enhance penetration of the peptides, and to provide a silky feel to formulations, e.g., methanoic acid, ethanoic acid, propanoic acid, butanoic acid, isobutyric acid, pentanoic acid, hexanoic acid, heptanoic acid, octanoic acid, nonanoic acid, decanoic acid, myristoleic acid, isovaleric acid, palmitoleic acid, sapienic acid, oleic acid, elaidic acid, vaccenic acid, linoleic acid, linoelaidic acid, a-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, medium chain fatty acids, e.g., C₆₋₁₂ fatty acids, or the like. Typical amounts when employed in topical compositions are from 1% by weight to 4% by weight. Other components can include anti-inflammatory agents, antioxidants, and solubility enhancers. For example, certain components of the composition tend to be difficult to solubilize in conventional formulations. Phosphatidylserine and oleuropein are known to exhibit solubility issues. In some embodiments, a siloxane polymer, e.g., caprylyl methicone, is used to solubilize phosphatidylserine. In some embodiments, caprylyl methicone is used to solubilize phosphatidylserine in anhydrous formulations. In some embodiments, panthenyl triacetate and naringenin is used to solubilize oleuropein. For topical compositions containing from about 0.05% by weight to about 0.1% by weight phosphatidylserine and/or from about 0.05% by weight to about 0.1% by weight oleuropein, caprylyl methicone in an amount of from about 0.5% by weight to about 1% by weight of caprylyl methicone can solubilize phosphatidylserine in an anhydrous formulation.

Bentonite and Other Clays

Bentonite clays can be employed in conjunction with the peptides to provide impart penetration and adsorption properties to the compositions, and can aid in stabilizing emulsions. Other clays, such as hectorite and magnesium aluminum silicate can also be employed. Bentonite or other clays can be modified to yield an organic modified clay compound. Salts (e.g., quaternary ammonium salts) of fatty acids (e.g., hydrogenated fatty acids) can be reacted with hectorite or other clays. As provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δn, where n indicates the lower numbered carbon of each pair of double-bonded carbon atoms. A shorthand notation specifying total # carbons: # double bonds, Δ_{double bond positrons} can be employed. For example, 20:4Δ_{5,8,11,14} refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-Δ9-octadecenoate) is a monounsaturated fatty acid, linolenate (all-cis-Δ9,12,15-octadecatrienoate) is a polyunsaturated fatty acid. Fatty acids suitable for use can comprise from 5 to 30 carbon atoms, e.g., 5, 6, 7, 8, 9, 10, 11, 12, 13, 14, 15, 16, 17, 18, 19, 20, 21, 22, 23, 24, 25, 26, 27, 28, 29, or 30 carbon atoms. The fatty acid can be fully saturated, or can include as many double bonds as are feasible for the chain length. Fatty acids suitable for functionalizing hectorite or other clays include palmitic acid and stearic acid. Dialkyl quaternary cationic modifiers include dipalmoyldimonium chloride and di stearyldimonium chloride. Amidoamine quaternary cationic modifiers include palmitamidopropyltrimonium chloride cetearyl alcohol and palmitamidopropyltrimonium chloride.

Anti-Irritation Agents

Panthenyl triacetate/naringenin are natural plant extracts that reduce redness and water loss through the skin. Typical amounts for anti-irritation agents when employed in topical compositions are from 1% by weight to 4% by weight.

Anti-Inflammatory Agents

Arnica montana extract includes components such as essential oils, fatty acids, thymol, pseudoguaianolide sesquiterpene lactones and flavanone glycosides. It can exhibit an anti-inflammatory effect. Typical amounts for anti-inflammatory agents when employed in topical compositions are from 1% by weight to 4% by weight.

Antioxidant Agents

Dunaliella salina extract includes components such as beta carotenes. It can exhibit an antioxidant effect. Typical amounts for anti-inflammatory agents when employed in topical compositions are from 0.1% by weight to 2% by weight.

Solubility Enhancers

Certain components of the composition tend to be difficult to solubilize in conventional formulations. For example, phosphatidylserine and oleuropein are known to exhibit solubility issues. It has been found that a siloxane polymer, e.g., caprylyl methicone, is particularly effective at solubilizing these two components in anhydrous formulations. For topical compositions containing from about 0.05% by weight to about 0.1% by weight phosphatidylserine and/or from about 0.05% by weight to about 0.1% by weight oleuropein, caprylyl methicone in an amount of from about 0.5% by weight to about 1% by weight of caprylyl methicone can solubilize these components in an anhydrous formulation.

In some embodiments, the peptides can be in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulations include, without limitation, monoglycerides, diglycerides, sulfatides, lysolecithin, phospholipids, saponin, bile acids, and the like. The presence of such additional components can influence the physical state, solubility, stability, rate of release, rate of clearance, and penetration of active ingredients.

The compositions for topical administration comprise the peptide compositions as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In certain embodiments, a silicone elastomer (e.g., dimethicone crosspolymer) is employed to increase delivery and penetration of the peptides into the skin. An alternative to increasing molecular weight (as with silicone gums) or adding filler (as with silicone compounds) is to partially crosslink siloxane polymers and disperse this material in an appropriate silicone carrier fluid. The resulting dimethicone crosspolymers (also known as silicone elastomers in the personal care industry) differ from basic polydimethylsiloxane (PDMS) because of the cross-linking between the linear polymers. These materials can be employed in peptide formulations, and also offer benefits in scar treatment, periwound protection and enzyme delivery. In skin care applications, the aesthetics of silicone elastomers (including those with functional groups) and their ability to absorb various oils (e.g., with a dimethicone/vinyl dimethicone crosspolymer such as Dow Corning® 9506 Elastomer Powder) are two of the elastomer's desirable properties. Silicone elastomers have a skin feel different from any of the silicone fluids, described as “smooth”, “velvety” and “powdery”. It can be modified by controlling the amount of liquid phase in the formula, and therefore the degree of swelling. Due to their film-forming properties, dimethicone crosspolymers can be used as delivery systems for active ingredients such as the peptides described herein, or other formulation components such as oil-soluble vitamins and sunscreens. Sunscreens such as octyl methoxycinnamate can be more efficiently delivered from a formulation containing a silicone elastomer, producing a higher sun protection factor (SPF). Silicone elastomer blends can be used to enhance SPF in oil-in-water formulations containing organic sunscreens. For example, in testing conducted regarding SPF, the addition of 4% silicone elastomer blend to a suncare formulation containing organic sunscreens increased the SPF from 5.7 to 18. This property of the silicone elastomer allows the effectiveness of sunscreen agents in a formulation to be maximized while reducing the amount needed to achieve a desired SPF. As a result, formulation costs can be reduced along with potential irritation caused by sunscreen actives. Accordingly, a higher SPF can be achieved with the same amount of UV absorber, resulting in enhanced performance with no added formulation cost. Silicone elastomers can be produced from linear silicone polymers by a variety of crosslinking reactions, e.g., by a hydrosilylation reaction in which a vinyl group reacts with a silicon hydride. The general process involves linear silicone polymers with reactive sites along the polymer chain reacting with a cross-linker. The dimethicone crosspolymer can be produced either as a gel made of a suspension of elastomer particles swollen in a carrier fluid (e.g., a mixture of high molecular weight silicone elastomer in cyclopentasiloxane such as Dow Corning® 9040 Silicone Elastomer Blend), or as a spray-dried powder (a dimethicone/vinyl dimethicone crosspolymer such as Dow Corning® 9506 Elastomer Powder). The gel form having desirable attributes is cyclomethicone, but low viscosity dimethicones and organic fluids can also be used. Examples of dimethicone crosspolymers in the suspension or gel form are high molecular weight silicone elastomer (12%) in decamethylcyclopentasiloxane (e.g., Dow Corning® ST-Elastomer 10) and a mixture of high molecular weight silicone elastomer in cyclopentasiloxane (e.g., Dow Corning® 9040 Silicone Elastomer Blend), which typically have an elastomer content ranging from 10 to 20% by weight.

The pharmaceutical excipients used in the topical preparations of the peptide compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Excipients can include a nonaqueous or aqueous carrier, and one or more agents selected from moisturizing agents, pH adjusting agents, deodorants, fragrances, chelating agents, preservatives, emulsifiers, thickeners, solubilizing agents, penetration enhancers, anti-irritants, colorants, surfactants, beneficial agents, pharmaceutical agents, and other components as known in the art for use in connection with topical compositions for treatment of the skin. In some embodiments, the composition is an aqueous composition. In some embodiments, the composition is an anhydrous composition to prevent skin irritation such as water-based irritant contact dermatitis or stinging sensation upon application to damaged skin. In some embodiments, the composition is formulated such that preservatives need not be employed (e.g., a preservative-free composition) so as to avoid skin irritation associated with certain preservatives.

Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for hydrophobic topical formulations include mineral oils, vegetable oils, and silicone oils. If desired, the peptide compositions as described herein may be dissolved or dispersed in a hydrophobic oil phase, and the oil phase may then be emulsified in an aqueous phase comprising water, alone or in combination with lower alcohols, glycerin, and/or glycols. It is generally preferred to employ anhydrous compositions, as the presence of water can result in stinging upon administration to skin tissues subject to laser treatment, chemical peel, dermabrasion, or the like. Anhydrous formulations may also act to prevent the development of water-based irritant contact dermatitis in damaged or sensitive skin, which may produce rashes and skin irritation that may retard wound healing and improvement in skin quality. Tsai, T. F., Maibach, H. I. How irritant is water? An overview. Contact Dermatitis 41(6) (1999): 311-314 (describing contact dermatitis caused by water as an irritant). However, in certain embodiments it may be acceptable to provide water based compositions, or to permit a limited amount of water to be present. For example, water may be present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the formulations as described herein can be advantageously employed where it is desirable to minimize osmotic shock.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical formulations include anionic, cationic, nonionic, and zwitterionic surfactants. Preferred ionic emulsifiers include phospholipids, such as lecithin and derivatives.

Lecithin and other phospholipids may be used to prepare liposomes containing the peptide compositions as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the peptide compositions as described herein.

In some embodiments, liposomes are used to prepare one or more peptides. In some embodiments, the peptide is hexapeptide-11. In some embodiments, the peptide is functionalized with an acetyl group.

In some embodiments, the liposomes comprise propanediol, lecithin, or a combination thereof. In some embodiments, the propanediol is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the propanediol is provided in a range of about 0.001% to about 6%, about 0.002% to about 4%, about 0.01% to about 3%, or about 0.02% to about 2% by weight. In some embodiments, the lecithin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the lecithin is provided in a range of about 0.001% to about 6%, about 0.002% to about 4%, about 0.01% to about 3%, or about 0.02% to about 2% by weight. In some embodiments, the liposomes comprise propanediol and lecithin. In some embodiments, the propanediol and lecithin are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt.) In some embodiments, the propanediol and lecithin are provided in a range of about 0.001% to about 6%, about 0.002% to about 4%, about 0.01% to about 3%, or about 0.02% to about 2% by weight. In some embodiments, the propanediol and lecithin are provided at about 0.90% by weight.

The topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in topical formulations include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the formulation. It is generally observed that the anhydrous formulations of the embodiments exhibit satisfactory stability, such that a preservative can be omitted from the formulation.

Suitable chelating agents for use in topical formulations include ethylene diamine tetraacetic acid, alkali metal salts thereof alkaline earth metal salts thereof, ammonium salts thereof, and tetraalkyl ammonium salts thereof.

The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The peptide compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the formulation, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

When the peptide formulations of the embodiments are administered by subcutaneous injection, it is preferably in the form of a pyrogen-free, parenterally acceptable aqueous solution or oleaginous suspension, emulsion or solution. Suspensions can be formulated according to methods well known in the art using suitable dispersing or wetting agents and suspending agents. The preparation of acceptable aqueous or nonaqueous solutions with suitable properties, e.g., pH, isotonicity, stability, and the like, is within the skill in the art. For example, an isotonic vehicle such as 1,3-butanediol, water, isotonic sodium chloride solution, Ringer's solution, dextrose solution, dextrose and sodium chloride solution, lactated Ringer's solution, or other vehicles as are known in the art can be employed, or a fixed oil can be employed conventionally as a solvent or suspending medium, e.g., synthetic mono or diglycerides, fatty acids, or the like. The peptide formulations can also contain stabilizers, preservatives, buffers, antioxidants, or other additives known to those of skill in the art.

In certain embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In certain embodiments, the addition of emollients, emulsion stabilizers, moisturizers, excipients, and other compounds may be modified to enhance the sensory properties of the topical compositions, including but not limited to: skin feel (silkiness, lightness, creaminess, etc.), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

To facilitate application, the composition may be provided as an ointment, an oil, a lotion, a paste, a powder, a gel, or a cream. The composition may also include additional ingredients such as a protective agent, an emollient, an astringent, a humectant, a sun screening agent, a sun tanning agent, a UV absorbing agent, an antibiotic agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an additional antioxidant agent, a chemotherapeutic agent, an anti-histamine agent, a vitamin or vitamin complex, a hormone, an anti-dandruff agent, an anti-wrinkle agent, an anti-skin atrophy agent, a skin whitening agent, a cleansing agent, additional peptides, additional modified peptides, and combinations thereof. In a further embodiment, the composition may avoid animal or cellular-based materials to avoid skin irritation. The composition can be applied to the dermis, or to mucous membranes.

Some embodiments include administering peptide compositions provided herein in topical compositions; however, other routes of administration are also contemplated (e.g., mucosal, subdermal, oral, or the like). Contemplated routes of administration include but are not limited to topical, mucosal, and subcutaneous. Suitable liquid forms include suspensions, emulsions, solutions, and the like. Unit dosage forms can also be provided, e.g., individual packets with a premeasured amount of the composition, configured for administration to the face or other body part on a predetermined schedule pre-procedure and post-procedure. Unit dosage forms configured for administration twice or three times a day pre-procedure and post-procedure are particularly preferred; however, in certain embodiments it can be desirable to configure the unit dosage form for administration once a day, four times a day, or more.

Compositions and formulations for topical administration can include transdermal patches, ointments, lotions, creams, gels, drops, sprays, liquids, aerosols, and powders. Conventional pharmaceutical carriers, aqueous, powder or oily bases, thickeners and the like may be employed. In certain applications, an ointment, lotion, cream, gel or similar formulation can be provided that can be applied to the skin using the fingers. Such formulations are typically provided in a squeeze tube or bottle or a pot, or in a roll-on, wherein a ball is secured in the top of a container of the formulation, wherein the ball is permitted to roll. By rolling the ball over the skin surface, liquid in the container is transferred to the skin in a controlled manner. An alternative delivery mechanism includes a container with a perforated lid with a mechanism for advancing an extrudable formulation through the lid. In another form, a gel formulation with sufficient structural integrity to maintain its shape is provided, which is advanced up a tube and applied to the skin (e.g., in a stick form). An advantage of the stick form is that only the formulation contacts the skin in the application process, not the fingers or a portion of a container. A liquid or gel can also be placed using an applicator, e.g., a wand, a sponge, a syringe, or other suitable method.

### Stability Testing

Stability testing of the topical formulations can be conducted as follows.

High temperature testing is now commonly used as a predictor of long-term stability. High temperature testing can be conducted at 37° C. (98F) and 45° C. (113° F.). If a product is stored at 45° C. for three months (and exhibits acceptable stability) then it should be stable at room temperature for two years. A good control temperature is 4° C. (39° F.) where most products will exhibit excellent stability. Sometimes, the product is also be subjected to −10° C. (14° F.) for three months.

In one embodiment, the product pass three cycles of temperature testing from −10° C. (14° F.) to 25° C. (77° F.). The product is placed at −10° C. for 24 hours and place it at room temperature (25° C.) for 24 hours. This completes one cycle. If the product passes three cycles then you can have a good degree of confidence in the stability of the product. An even more rigorous test is a −10° C. to 45° C. five-cycle test. This puts emulsions under a tremendous stress and, if it passes the test, indicates that you have a highly stable product.

The dispersed phase (of an oil-in-water emulsion) has a tendency to separate and rise to the top of the emulsion forming a layer of oil droplets. This phenomenon is called creaming. Creaming is one of the first signs of impending emulsion instability. A test method to predict creaming is centrifugation. Heat the emulsion to 50° C. (122° F.) and centrifuge it for thirty minutes at 3000 rpm. Then inspect the resultant product for signs of creaming.

Both formulas and packaging can be sensitive to the UV radiation. The product is placed in glass and the actual package in a light box that has a broad-spectrum output. Another glass jar completely covered with aluminum foil serves as a control. Discoloration of the product may be observed.

For all the above mentioned tests the color, odor / fragrance, viscosity, pH value, and, if available, particle size uniformity and/or particle agglomeration under the microscope can be observed.

Kits for Non-Invasive Use and Use with Invasive Procedures

Some embodiments of the methods and compositions provided herein include kits comprising peptides provided herein. In some embodiments, kits can be provided to an administering physician, other health care professional, a patient, or a caregiver. In some embodiments, a kit comprises a container which contains the peptide compositions in a suitable topical formulation, and instructions for administering the peptide composition to a subject. The kit can optionally also contain one or more additional therapeutic or other agents. For example, a kit containing a peptide composition in topical form can be provided along with other skin care agents, such as, cleansers, occlusive moisturizers, penetrating moisturizers, sunscreens, sunblocks, and the like. The kit may contain the peptide composition in bulk form, or can contain separate doses of the peptide composition for serial or sequential administration. The kit can optionally contain one or more diagnostic tools, administration tools, and/or instructions for use. The kit can contain suitable delivery devices, such as, syringes, pump dispensers, single dose packets, and the like, along with instructions for administering the peptide compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

In some embodiments, the formulation is configured to support the skin before, during and after cosmetic procedures, and also works with the skin's own natural regenerating process and assists in improving the skin's appearance, and skin tightness. The topical formulation can be applied immediately post-procedure for faster recovery, or generally for healthier looking skin. The formulation can increase natural levels of elastin in the skin, improves the quality of existing elastin, stimulates increase in collagen production, and exhibits high antioxidant activity to reduce inflammation, redness and irritation. The topical formulation is suitable for all skin types and post-procedure skin. The topical formulations can be provided to the patient in bulk form, to permit a suitable amount of the peptides to be self-administered by the patient. For example, the patient can apply an amount of the formulation sufficient to provide an even coating over the affected area or as otherwise instructed by the physician. In certain embodiments it can desirable to incorporate additional therapeutic or active agents into the topical formulation. Alternatively, adjunct therapies or agents can be administered separately. For example, a cleanser, a sunblock, a sunscreen, a penetrating moisturizer, and/or an occlusive moisturizer can be provided for administration before or after the topical composition of the embodiments.

In one embodiment, a kit is provided for use in connection with an invasive skin procedure, as described herein. The kit, termed “an invasive kit”, includes a topical peptide composition, an occlusive moisturizer, a gentle cleanser, a penetrating moisturizer, and a broad spectrum SPF 30+ sunscreen.

In another embodiment, a kit is provided for use in connection with improving skin health but not in connection with an invasive skin procedure. The kit, termed “a noninvasive kit,” in some embodiments, includes a topical peptide composition, a gentle cleanser, a penetrating moisturizer, and a broad spectrum SPF 30+ sunscreen.

The various examples of creams, ointments, lotions, solutions, gels, sprays and patches may incorporate the peptide compositions as described herein as the active ingredient, in combination with penetration enhancing agents and other active agents acting synergistically on the skin for the promotion of wound healing or wound closure or the treatment of chronic cutaneous wound.

### NUMBERED EMBODIMENTS

Numbered embodiment 1 comprises a method for improving wound healing after a skin procedure or surgical procedure in a subject comprising: (a) applying to a skin region of the subject a first topical composition comprising a tripeptide-1 and hexapeptide-12 before the skin procedure or the surgical procedure; and (b) applying to the skin region of the subject a second topical composition comprising tripeptide-1, a hexapeptide-12, and a hexapeptide-11, wherein the first topical composition, the second topical composition, or both is administered in an amount sufficient to modulate an expression level of one or more inflammatory or regenerative genes. Numbered embodiment 2 comprises the method of numbered embodiment 1, wherein step (b) further comprises applying the first topical composition. Numbered embodiment 3 comprises the method of numbered embodiments 1-2, wherein the first topical composition is administered for at least one week before the surgical procedure. Numbered embodiment 4 comprises the method of numbered embodiments 1-3, wherein the first topical composition is administered for at least two weeks before the surgical procedure. Numbered embodiment 5 comprises the method of numbered embodiments 1-4, wherein the second topical composition is administered for at least two weeks after the surgical procedure. Numbered embodiment 6 comprises the method of numbered embodiments 1-5, wherein the second topical composition is administered for at least ten weeks after the surgical procedure. Numbered embodiment 7 comprises the method of numbered embodiments 1-6, wherein the first topical composition, the second topical composition, or both is administered one, two, three, four, five, or six times a day. Numbered embodiment 8 comprises the method of numbered embodiments 1-7, wherein the first topical composition is administered at least two times a day for at least two weeks before the skin procedure or the surgical procedure and the second topical composition is administered at least two times a day for at least two weeks after the skin procedure or the surgical procedure. Numbered embodiment 9 comprises the method of numbered embodiments 1-8, wherein the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof. Numbered embodiment 10 comprises the method of numbered embodiments 1-9, wherein the one or more inflammatory or regenerative genes encodes a chemokine receptor, a chemokine ligand, an angiotensin receptor, a complement component, a cholinergic receptor, a clusterin, a cytochrome, a fibroblast growth factor, a hepatocyte growth factor, an interleukin receptor, an interleukin, an integrin, a killer cell like lectin receptor, a leukotriene synthase, a lymphocyte antigen, a nuclear factor of activated T-cell, a Nod-like receptor (NLR), a phospholipase, a serpin, a single Ig IL-1-related receptor, a sialic acid binding Ig like lectin, a tachykinin receptor, a TNF receptor, a tyrosylprotein sulfotransferase, or a fragment or variant thereof. Numbered embodiment 11 comprises the method of numbered embodiments 1-10, wherein the one or more inflammatory or regenerative genes encodes a chemokine-like receptor, a chemokine ligand, a cluster of differentiation ligand, a cholinergic receptor, a fms related tyrosine kinase ligand, a growth differentiation factor, an interleukin, an interleukin receptor, a lymphocyte antigen, a matrix metallopeptidase, a Nod-like receptor (NLR), a phospholipase, a signal peptide, a CUB domain and EGF like domain, a sialophorin, a transglutaminase, or a fragment or variant thereof. Numbered embodiment 12 comprises the method of numbered embodiments 1-11, wherein the one or more inflammatory or regenerative genes comprises ACKR4, AGTR1, C1R, C1S C3, CCL7,CCL20, CCRL1, CCRL2, CFD, CHRNA7, CLU, CYBB, FIGF, HGF, IL21R,IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF I3B, TNFSF15, or TPST1. Numbered embodiment 13 comprises the method of numbered embodiments 1-12, wherein the administration of the topical composition modulates two or more inflammatory or regenerative genes comprising ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF13B, TNFSF15, or TPST1. Numbered embodiment 14 comprises the method of numbered embodiments 1-13, wherein the one or more inflammatory or regenerative genes comprises AGTR1, C1R, C3, CCL20, CCRL2, CLU, CYBB, FIGF, IL21R, ITGB2, KLRG1, LTC4S, LY86, NFAM1, NFATC4, NLRP3, PLCB2, SERPINA1, SIGLEC1, or TNFSF13B. Numbered embodiment 15 comprises the method of numbered embodiments 1-14, wherein the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. Numbered embodiment 16 comprises the method of numbered embodiments 1-15, wherein the administration of the topical composition modulates two or more inflammatory or regenerative genes comprising AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. Numbered embodiment 17 comprises the method of numbered embodiments 1-16, wherein the one or more inflammatory or regenerative genes comprises IL6, PLA2G4C, TNFRSF4, 77VFSFI5, or TPST1. Numbered embodiment 18 comprises the method of numbered embodiments 1-17, wherein the one or more inflammatory or regenerative genes comprises IL6. Numbered embodiment 19 comprises the method of numbered embodiments 1-18, wherein the one or more inflammatory or regenerative genes comprises C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. Numbered embodiment 20 comprises the method of numbered embodiments 1-19, wherein the administration of the topical composition modulates two or more inflammatory or regenerative genes comprising C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. Numbered embodiment 21 comprises the method of numbered embodiments 1-20, wherein the one or more inflammatory or regenerative genes comprises C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. Numbered embodiment 22 comprises the method of numbered embodiments 1-21, wherein the administration of the topical composition modulates two or more inflammatory or regenerative genes comprising C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. Numbered embodiment 23 comprises the method of numbered embodiments 1-22, wherein the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. Numbered embodiment 24 comprises the method of numbered embodiments 1-23, wherein the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. Numbered embodiment 25 comprises the method of numbered embodiments 1-24, wherein the one or more inflammatory or regenerative genes comprises C1R or SCUBEJ. Numbered embodiment 26 comprises the method of numbered embodiments 1-25, wherein the one or more inflammatory or regenerative genes comprises TGM2 or PLCB2. Numbered embodiment 27 comprises the method of numbered embodiments 1-26, wherein the expression level is increased by at least 1.25-fold as compared to control. Numbered embodiment 28 comprises the method of numbered embodiments 1-27, wherein the expression level is increased by at least 1.5-fold as compared to control. Numbered embodiment 29 comprises the method of numbered embodiments 1-28, wherein the expression level is increased by at least 2-fold as compared to control. Numbered embodiment 30 comprises the method of numbered embodiments 1-29, wherein the expression level is increased by at least 3-fold as compared to control. Numbered embodiment 31 comprises the method of numbered embodiments 1-30, wherein the control is a skin region that does not receive the first topical composition, the second topical composition, or both. Numbered embodiment 32 comprises the method of numbered embodiments 1-31, wherein the control is a baseline expression level. Numbered embodiment 33 comprises the method of numbered embodiments 1-32, wherein the expression level is increased after at least about 2 weeks. Numbered embodiment 34 comprises the method of numbered embodiments 1-33, wherein the expression level is increased after at least about 4 weeks. Numbered embodiment 35 comprises the method of numbered embodiments 1-34, further comprising, subsequent to administration of the topical composition, detecting the expression level of the at least one gene by contacting a sample obtained from the treated skin region of the subject with a probe that recognizes the at least one gene and detect binding between the at least one gene and the probe. Numbered embodiment 36 comprises the method of numbered embodiments 1-35, further comprising, before administration of the topical composition, detecting expression level of the one or more inflammatory or regenerative genes comprising ACKR4, AGTR1, C1R, C1S C3, CCL17, CCL19, CCL20, CCL22, CCRL1, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B by contacting a skin sample of the subject with a probe that recognizes the one or more inflammatory or regenerative genes comprising ACKR4, AGTR1, C1R, C1S C3, CCL17, CCL19, CCL20, CCL22, CCRL1, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B and detect binding between the gene and the probe. Numbered embodiment 37 comprises the method of numbered embodiments 1-36, wherein the tripeptide-1 comprises palmitoyl tripeptide-1, myristoyl tripeptide-1, or a combination thereof. Numbered embodiment 38 comprises the method of numbered embodiments 1-37, wherein the tripeptide-1 is present at 1-10 ppm. Numbered embodiment 39 comprises the method of numbered embodiments 1-38, wherein the hexapeptide-11 comprises palmitoyl hexapeptide-11, myristoyl hexapeptide-11, or a combination thereof. Numbered embodiment 40 comprises the method of numbered embodiments 1-39, wherein the hexapeptide-11 is present at 0.001-1 ppm Numbered embodiment 41 comprises the method of numbered embodiments 1-40, wherein the hexapeptide-12 comprises palmitoyl hexapeptide-12, myristoyl hexapeptide-12, or a combination thereof. Numbered embodiment 42 comprises the method of numbered embodiments 1-41, wherein the hexapeptide-12 is present at 0.5-10 ppm. Numbered embodiment 43 comprises the method of numbered embodiments 1-42, wherein the second topical composition further comprises a tetrapeptide. Numbered embodiment 44 comprises the method of numbered embodiments 1-43, wherein the tetrapeptide is tetrapeptide-2. Numbered embodiment 45 comprises the method of numbered embodiments 1-44, wherein the tetrapeptide is present at 1-10 ppm. Numbered embodiment 46 comprises the method of numbered embodiments 1-45, wherein the first topical composition comprises phosphatidylserine and oleuropein. Numbered embodiment 47 comprises the method of numbered embodiments 1-46, wherein the phosphatidylserine is present in a range of about 0.005 weight (wt.) % to about 0.1 wt. %. Numbered embodiment 48 comprises the method of numbered embodiments 1-47, wherein the phosphatidylserine is present at no more than 5.0 wt. %. Numbered embodiment 49 comprises the method of numbered embodiments 1-48, wherein the oleuropein is present at no more than 0.050 wt. %. Numbered embodiment 50 comprises the method of numbered embodiments 1-49, wherein the second topical composition further comprises phosphatidylserine. Numbered embodiment 51 comprises the method of numbered embodiments 1-50, wherein the phosphatidylserine is present in a range of about 0.005 wt. % to about 0.1 wt. %. Numbered embodiment 52 comprises the method of numbered embodiments 1-51, wherein the phosphatidylserine is present at no more than 5.0 wt. %. Numbered embodiment 53 comprises the method of numbered embodiments 1-52, wherein the first topical composition, the second topical composition, or both further comprises ceramide NP, Tremella fuciformis extract, niacinamide, hydrogenated lecithin, C12-16 alcohols, palmitic acid, avocado extract, shea butter, bentonite, phytoene/phytofluene, hydroxymethoxyphenyl decanone, polyholosides, Plantago lanceolata, dill extract, hydrolyzed Candida saitoana extract, Centella asiatica, propanediol, lecithin, Euglena gracilis extract, aqua, caffeine, Glaucium flavum leaf extract, or combinations thereof. Numbered embodiment 54 comprises the method of numbered embodiments 1-53, wherein the first topical composition, the second topical composition, or both is an aqueous topical composition. Numbered embodiment 55 comprises the method of numbered embodiments 1-54, wherein the first topical composition, the second topical composition, or both is an anhydrous topical composition. Numbered embodiment 56 comprises the method of numbered embodiments 1-55, wherein the subject is a human. Numbered embodiment 57 comprises the method of numbered embodiments 1-56, wherein the skin procedure comprises a laser treatment, a chemical peel, microdermabrasion, microneedling, or radiofrequency microneedling. Numbered embodiment 58 comprises the method of numbered embodiments 1-57, wherein the surgical procedure comprise a panniculectomy, liposuction, a lower body lift, brachioplasty, an inner thigh lift, a buttock augmentation, a circumferential body lift, a breast lift, a breast reduction, a breast augmentation, or a labiaplasty. Numbered embodiment 59 comprises the method of numbered embodiments 1-58, wherein the method accelerates the healing process, accelerates clearance of products including lipid particles, accelerates resolution of inflammation stimulates extracellular matrix remodeling, reduces induration, reduces fibrous banding, reduces pain or discomfort, or combinations thereof. Numbered embodiment 60 comprises method for improving wound healing in a subject comprising applying to a skin region of the subject a topical composition comprising a tripeptide-1, a hexapeptide-12, and a hexapeptide-11, wherein the topical composition is administered in an amount sufficient to modulate an expression level of one or more inflammatory or regenerative genes, and wherein the topical composition is administered before a procedure, after a procedure, or both, and wherein the procedure is a surgical skin removal procedure or a procedure to remove fat and skin. Numbered embodiment 61 comprises the method of numbered embodiments 1-60, wherein the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof. Numbered embodiment 62 comprises the method of numbered embodiments 1-61, wherein the one or more inflammatory or regenerative genes encodes a chemokine receptor, a chemokine ligand, an angiotensin receptor, a complement component, a cholinergic receptor, a clusterin, a cytochrome, a fibroblast growth factor, a hepatocyte growth factor, an interleukin receptor, an interleukin, an integrin, a killer cell like lectin receptor, a leukotriene synthase, a lymphocyte antigen, a nuclear factor of activated T-cell, a Nod-like receptor (NLR), a phospholipase, a serpin, a single Ig IL-1-related receptor, a sialic acid binding Ig like lectin, a tachykinin receptor, a TNF receptor, a tyrosylprotein sulfotransferase, or a fragment or variant thereof. Numbered embodiment 63 comprises the method of numbered embodiments 1-62, wherein the one or more inflammatory or regenerative genes encodes a chemokine-like receptor, a chemokine ligand, a cluster of differentiation ligand, a cholinergic receptor, a fms related tyrosine kinase ligand, a growth differentiation factor, an interleukin, an interleukin receptor, a lymphocyte antigen, a matrix metallopeptidase, a Nod-like receptor (NLR), a phospholipase, a signal peptide, a CUB domain and EGF like domain, a sialophorin, a transglutaminase, or a fragment or variant thereof. Numbered embodiment 64 comprises the method of numbered embodiments 1-63, wherein the one or more inflammatory or regenerative genes comprises ACKR4, AGTR1, C1R, C1S C3, CCL17, CCL20, CCRL1, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF I3B, TNFSF15, or TPST1. Numbered embodiment 65 comprises the method of numbered embodiments 1-64, wherein the administration of the topical composition modulates two or more inflammatory or regenerative genes comprising ACKR4, AGTR1, C1R, C1S C3, CCL17, CCL20, CCR19, CCRL2, CFD, CHRNA7, CLU, CYBB, FIGF, HGF, IL21R, IL33, IL3RA, IL6, ITGB2, KLRG1, LTC4S, LY86, NFAM1, NFATC4, NLRP3, PLA2G4C, PLCB2, SERPINA1, SIGIRR, SIGLEC1, TACR1, TNFRSF4, TNFSF I3B, TNFSF15, or TPST1. Numbered embodiment 66 comprises the method of numbered embodiments 1-65, wherein the one or more inflammatory or regenerative genes comprises AGTR1, C1R, C3, CCL20, CCRL2, CLU, CYBB, FIGF, IL21R, ITGB2, KLRG1, LTC4S, LY86, NFAM1, NFATC4, NLRP3, PLCB2, SERPINA1, SIGLEC1, or TNFSF I3B. Numbered embodiment 67 comprises the method of numbered embodiments 1-66, wherein the one or more inflammatory or regenerative genes comprises AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF13B. Numbered embodiment 68 comprises the method of numbered embodiments 1-67, wherein the administration of the topical composition modulates two or more inflammatory or regenerative genes comprising AGTR1, C1R, C3, CCL20, CCRL2, CLU, CYBB, FIGF, IL21R, ITGB2, KLRG1, LY86, NLRP3, PLCB2, SERPINA1, SIGLEC1, or TNFSF3B. Numbered embodiment 69 comprises the method of numbered embodiments 1-68, wherein the one or more inflammatory or regenerative genes comprises IL6, PLA2G4C, TNFRSF4, TNFSF15, or TPST1. Numbered embodiment 70 comprises the method of numbered embodiments 1-69, wherein the one or more inflammatory or regenerative genes comprises IL6. Numbered embodiment 71 comprises the method of numbered embodiments 1-70, wherein the one or more inflammatory or regenerative genes comprises C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. Numbered embodiment 72 comprises the method of numbered embodiments 1-71, wherein the administration of the topical composition modulates two or more inflammatory or regenerative genes comprising C1R, CCL17, CCL19, CCL20, CCL22, CCRL2, CD40LG, CHRNA7, EBI3, FLT3LG, GDF15, IL11, IL21R, IL27RA, IL32, LY86, MMP25, NLRP12, PLCB2, SCUBE1, SPN, or TGM2. Numbered embodiment 73 comprises a method of numbered embodiments 1-72, wherein the one or more inflammatory or regenerative genes comprises C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. Numbered embodiment 74 comprises a method of numbered embodiments 1-73, wherein the administration of the topical composition modulates two or more inflammatory or regenerative genes comprising C1R, CCL17, CCL19, CCL20, CCL22, CCRL2, CD40LG, EBI3, FLT3LG, GDF15, IL21R, IL27RA, IL32, LY86, MMP25, NLRP12, PLCB2, SCUBE1, SPN, or TGM2. Numbered embodiment 75 comprises a method of numbered embodiments 1-74, wherein the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. Numbered embodiment 76 comprises a method of numbered embodiments 1-75, wherein the one or more inflammatory or regenerative genes comprises CCL17, CCL19, CCL20, CCL22, CCRL2, CD40LG, EBI3, FLT3LG, GDF15, IL21R, IL27RA, NLRP12, or SPN. Numbered embodiment 77 comprises a method of numbered embodiments 1-76, wherein the one or more inflammatory or regenerative genes comprises C1R or SCUBE1. Numbered embodiment 78 comprises a method of numbered embodiments 1-77, wherein the one or more inflammatory or regenerative genes comprises TGM2 or PLCB2. Numbered embodiment 79 comprises a method of numbered embodiments 1-78, wherein the expression level is increased by at least 1.25-fold as compared to control. Numbered embodiment 80 comprises a method of numbered embodiments 1-79, wherein the expression level is increased by at least 1.5-fold as compared to control. Numbered embodiment 81 comprises a method of numbered embodiments 1-80, wherein the expression level is increased by at least 2-fold as compared to control. Numbered embodiment 82 comprises a method of numbered embodiments 1-81, wherein the expression level is increased by at least 3-fold as compared to control. Numbered embodiment 83 comprises a method of numbered embodiments 1-82, wherein the control is a skin region that does not receive the topical composition. Numbered embodiment 84 comprises a method of numbered embodiments 1-83, wherein the control is a baseline expression level. Numbered embodiment 85 comprises a method of numbered embodiments 1-84, wherein the expression level is increased after at least about 2 weeks. Numbered embodiment 86 comprises a method of numbered embodiments 1-85, wherein the expression level is increased after at least about 4 weeks. Numbered embodiment 87 comprises a method of numbered embodiments 1-86, further comprising, subsequent to administration of the topical composition, detecting the expression level of the at least one gene by contacting a sample obtained from the treated skin region of the subject with a probe that recognizes the at least one gene and detect binding between the at least one gene and the probe. Numbered embodiment 88 comprises a method of numbered embodiments 1-87, further comprising, before administration of the topical composition, detecting expression level of the one or more inflammatory or regenerative genes comprising ACKR4, AGTR1, C1R, C1S C3, CCL17, CCL19, CCL20, CCL22, CCRL1, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B by contacting a skin sample of the subject with a probe that recognizes the one or more inflammatory or regenerative genes comprising ACKR4, AGTR1, C1R, C1S C3, CCL17, CCL19, CCL20, CCL22, CCR19, CCRL2, CD40LG, CFD, CHRNA7, CLU, CYBB, EBI3, FIGF, FLT3LG, GDF15, HGF, IL11, IL21R, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, or TNFSF13B and detect binding between the gene and the probe. Numbered embodiment 89 comprises a method of numbered embodiments 1-88, wherein the tripeptide-1 comprises palmitoyl tripeptide-1, myristoyl tripeptide-1, or a combination thereof. Numbered embodiment 90 comprises a method of numbered embodiments 1-89, wherein the tripeptide-1 is present at 1-10 ppm. Numbered embodiment 91 comprises a method of numbered embodiments 1-90, wherein the hexapeptide-11 comprises palmitoyl hexapeptide-11, myristoyl hexapeptide-11, or a combination thereof. Numbered embodiment 92 comprises a method of numbered embodiments 1-91, wherein the hexapeptide-11 is present at 0.001-1 ppm Numbered embodiment 93 comprises a method of numbered embodiments 1-92, wherein the hexapeptide-12 comprises palmitoyl hexapeptide-12, myristoyl hexapeptide-12, or a combination thereof. Numbered embodiment 94 comprises a method of numbered embodiments 1-93, wherein the hexapeptide-12 is present at 0.5-10 ppm. Numbered embodiment 95 comprises a method of numbered embodiments 1-94, further comprising a tetrapeptide. Numbered embodiment 96 comprises a method of numbered embodiments 1-95, wherein the tetrapeptide is tetrapeptide-2. Numbered embodiment 97 comprises a method of numbered embodiments 1-96, wherein the tetrapeptide is present at 1-10 ppm. Numbered embodiment 98 comprises a method of numbered embodiments 1-97, wherein the topical composition further comprises phosphatidylserine. Numbered embodiment 99 comprises a method of numbered embodiments 1-98, wherein the phosphatidylserine is present in a range of about 0.005 wt. % to about 0.1 wt. %. Numbered embodiment 100 comprises a method of numbered embodiments 1-99, wherein the phosphatidylserine is present at no more than 5.0 wt. %. Numbered embodiment 101 comprises a method of numbered embodiments 1-100, further comprising ceramide NP, Tremella fuciformis extract, niacinamide, hydrogenated lecithin, C12-16 alcohols, palmitic acid, avocado extract, shea butter, bentonite, phytoene/phytofluene, hydroxymethoxyphenyl decanone, polyholosides, Plantago lanceolata, dill extract, oleuropein, hydrolyzed Candida saitoana extract, Centella asiatica, propanediol, lecithin, Euglena gracilis extract, aqua, caffeine, Glaucium flavum leaf extract, or combinations thereof. Numbered embodiment 102 comprises a method of numbered embodiments 1-101, wherein the topical composition is an aqueous topical composition. Numbered embodiment 103 comprises a method of numbered embodiments 1-102, wherein the topical composition is an anhydrous topical composition. Numbered embodiment 104 comprises a method of numbered embodiments 1-103, wherein the topical composition is administered for at least two weeks before the procedure. Numbered embodiment 105 comprises a method of numbered embodiments 1-104, wherein the topical composition is administered for at least two weeks after the procedure. Numbered embodiment 106 comprises a method of numbered embodiments 1-105, wherein the topical composition is administered one, two three, four, five, or six times a day. Numbered embodiment 107 comprises a method of numbered embodiments 1-106, wherein the topical composition is administered one, two three, four, five, or six times a day for at least two weeks before the procedure and at least two weeks after the procedure. Numbered embodiment 108 comprises a method of numbered embodiments 1-107, wherein the method accelerates the healing process, accelerates clearance of products including lipid particles, accelerates resolution of inflammation stimulates extracellular matrix remodeling, reduces induration, reduces fibrous banding, reduces pain or discomfort, or combinations thereof. Numbered embodiment 109 comprises a method of numbered embodiments 1-108, wherein the subject is a human.

### EXAMPLES

### Example 1

### Formulation

A topical formulation was prepared comprising peptides in combination with excipients. The formulation so prepared was evaluated for suitability for use as a topical formulation, including skin feel and stability. The formulation was prepared as in the following table.

TABLE 1
Exemplary Formula 1 for the regenerating body complex product

| Ingredient | % by wt. |
|---|---|
| Hydrogenated Lecithin, | 1-6% |
| C12-16 Alcohols, Palmitic | |
| Acid | |
| Avocado extract, Shea | 0.25-2% |
| butter, Bentonite | |
| Acetyl Tetrapeptide-2 | 1-4% |
| Phytoene/Phytofluene | 0.2-1% |
| Hydroxymethoxyphenyl | 0.5-2% |
| Decanone- | |
| TriHex -Palmitoyl | 1-6% |
| Tripeptide-1 | |
| Palmitoyl Hexapeptide -12 | 0.25-4% |
| Polyholosides from flax | 2.5-10% |
| seeds | |
| Plantago lanceolata also | 1-4% |
| called “Plantain” | |
| Dill extract | 0.25-4% |
| Phosphatidylserine | 0.025-0.1% |
| Oleuropein | 0.01-0.05% |
| Hexapeptide-11 | 0.005-0.02% |
| Hydrolyzed Candida | 1-6% |
| Saitoana Extract | |
| Centella Asiatica | 0.25-4% |
| Propanediol, Lecithin | 0.25-2% |
| Euglena Gracilis Extract, | 0.05-1% |
| Aqua, Caffeine, Glaucium | |
| Flavum Leaf Extract | |

TABLE 2
Exemplary Formula 2 for the regenerating skin nectar

| Ingredient | % (wt.) |
|---|---|
| Cyclopentasiloxane, Dimethicone Crosspolymer | 80-90% |
| Heptyl Undecylenate | 1-5% |
| Panthenyl Triacetate/Naringenin | 1-5% |
| Arnica Montana Extract | 1-5% |
| Dunaliella Salina Extract | 0.25-2.5% |
| Carrier: | 1-5% |
| Pentaerythrityl tetraisostearate | |
| Caprylic/capric triglyceride | |
| Propylene carbonate | |
| Stearalkonium hectorite | |
| Palmitoyl hexapeptide-12 | |
| Carrier: | 1-5% |
| Pentaerythrityl tetraisostearate | |
| Caprylic/capric triglyceride | |
| Propylene carbonate | |
| Disteardimonium hectorite | |
| Palmitoyl tripeptide-1 | |
| Caprylyl methicone | 0.25-2.5% |
| Phospatidylserine/lecithin | 0.025-.25% |
| Oleuropein | 0.025-.25% |

* Present in formulation at 0.03 wt. %; present in carrier at 100 ppm.
** Present in formulation at 0.03 wt. %; present in carrier at 100 ppm.

### Example 2

### Inflammatory and Regenerative Gene Expression Following Liposuction

A split-body study was performed in order to compare comprehensive gene expression changes in subjects following bilateral extensive medial thigh liposuction with and without the use of a topical treatment.

Material and Methods

Study Population

This was a split-body, randomized, double-blinded study. A total of 6 subjects participated in this study. The eligible subjects included women undergoing extensive liposuction of the medial thighs. The participants agreed to apply the topical products twice daily for 2 weeks before the surgery and post-surgery. Subjects that were not good candidates for the surgery, as determined by the physician, were excluded from participating in the study. Pregnant or lactating subjects were excluded, as well as those planning on becoming pregnant during the study duration.

Liposuction Procedure

The liposuction ports were identical in each case. Two incisions on each leg. The superior incision along the bikini line in the upper medial groin and the second along the medial aspect of the thigh 10 cm superior to the medial condyle of the knee. The liposuction access was always remote from the skin biopsy sites. The liposuction volume was consistent at 250 cc per medial thigh with similar suction techniques.

Topical Application

The subjects were randomized to receive the regenerating skin nectar (RSN; Exemplary Formula 2) and the regenerating body complex product (RBP; Exemplary Formula 1) either on the right or left side of the treatment area. On the opposite side, they received a mild unscented moisturizer (Cetaphil® Lotion, Galderma Laboratories, Fort Worth, TX), which was applied from two separate bottles to unsure that the subjects were blinded to the experimental condition. The kits and patients numbers were randomized in excel, and the investigator was blinded. The subjects were preconditioned with RSN 2 weeks prior to the elected surgery on the designated side. Immediately after the procedure, they applied RSN and then RBP to the designated side, around the incision and procedure area for a up to 10 weeks or longer as determined by the physician. On the opposite side, the subjects applied the moisturizer 2 weeks before surgery and after the surgery for up to 10 weeks. Cetaphil was used as a comparative moisturizer. The Cetaphil was meant to counter the effects of the increased hydration that was caused by the use of the treatment products.

Punch Biopsies

Among the 6 subjects, 5 consented to punch biopsies (3 mm) in the surgical area of both treatment sides. 3 patient biopsies were subjected to gene expression analysis, while the remaining 2 were submitted for histological examination (one patient refused biopsies). The biopsy sites were located along a line drawn from the insertion of the adductor brevis on the inferior ramus of the pubis to the medial condyle of the femur at the knee. The first biopsy was 8 cm inferior to the adductor insertion, the second biopsy was 10 cm and the third was 12 cm inferior. The biopsies were performed at the pre-treatment stage and at weeks 2 and 4 postsurgery. All biopsies were performed by a board-certified plastic surgeon. Upon collection, the biopsies for gene expression were immediately placed in RNAlater (Invitrogen) stabilization solution and stored overnight at room temperature. Then, the samples were stored at −20₀C until they were shipped to Genemarkers (Kalamazoo, Mich.) for RNA processing. A total of 18 clinical skin biopsies were obtained from three patients assigned for gene expression studies.

RNA Isolation

RNA isolation and subsequent RNA analyses described below were performed by Genemarkers (Kalamazoo, Mich.). Briefly, total RNA was isolated from each biopsy using an RNeasy Mini kit (Qiagen, Germantown, Md.) following the manufacturer's instructions for fibrous tissues. RNA concentration and purity were determined using a Nanodrop 2000 spectrophotometer (ThermoFisher, Waltham, Mass.). RNA integrity was assessed using an Agilent Bioanalyzer 2100 (Santa Clara, Calif.). All samples showed high-quality RNA metrics and similar yields were obtained for all samples, with the exception of one sample. This sample had a low RNA yield and required vacuum concentration.

Endogenous Control Gene Selection

An endogenous control gene was selected that was consistently expressed in all of the samples for comparison. The Thermo Fisher (TF) Human Inflammation Panel includes 21 endogenous controls tested across 29 separate assays. Three algorithms (Normfinder algorithm, Minimum Variance Median algorithm (minvarmed), and the coefficient of variability (CV)) were used to calculate the stability scores in order to determine the most consistent endogenous control gene. Lower stability scores represented a more consistent expression between samples in the study. Based on the stability scores and average rankings of the endogenous controls, hypoxanthine phosphoribosyltransferase 1 (HPRT1) was identified as the most stable endogenous control gene.

Reverse Transcription (RT), Product Preparation and Preamplification

The TF Human Inflammation Panel contains 586 validated TaqMan Assays related to human inflammation. The RT and preamplification steps were carried out in an Applied Biosystems 2720 Thermal Cycler (Foster City, Calif.), according to the manufacturer's instructions for Low Sample Input on TaqMan OpenArray Pathway Panels (Life Technologies, Carlsbad, Calif.). Two separate gene-specific RT products were generated from 100 ng of each RNA sample using a SuperScript Vilo Reverse Transcription (RT) Kit (Invitrogen, Carlsbad, Calif.) and two custom Taqman® PreAmp Pools (Applied Biosystems), labeled pool A or pool B (10 minutes at 25° C., 60 minutes at 42° C., and 5 minutes at 85° C.). Each RT product was then preamplified using Taqman® Preamplification Master Mix (Applied Biosystems), the same custom Taqman® PreAmp Pool (A or B), and 14 cycles of preamplification (15 seconds at 95° C., 4 minutes at 60° C.). At the end of the preamplification, the products from pools A and B for each sample were mixed thoroughly and then diluted 1:20 with nuclease-free water.

qPCR Processing and Analysis

The qPCR reactions were performed in an OpenArray format in the Life Technologies QuantStudio 12K Flex instrument. Each gene was assayed in duplicate. The qPCR data quality was assessed and exported from the raw data files using Expression Suite software (Applied Biosystems). The qPCR data was then imported into the “OmicsOffice for qPCR” tool of TIBCO Spotfire Analyst software. Statistical data analysis was performed using the relative quantitation (RQ) method. In the first step of an RQ analysis, the cycle threshold (CT) value of the target gene was normalized to the CT value of an endogenous control gene to generate the delta CT (dCT). The dCT values were calculated to normalize the variability between the samples that might occur during the experimental procedures. (See the Endogenous Control Gene Selection section for more details).

SkinFibroMeter Measurements

The patients were assessed using SkinFibroMeter (Delfin Technologies, Miami, FL.) measurements. The SkinFibroMeter assesses induration in absolute units of stiffness. The subjects underwent a pre-treatment visit, a baseline (Day 0) visit and follow-up visits at every week initially then at 2 weeks and then every 3 weeks until 10 weeks post procedure/surgery. An increased value over the baseline reading was indicative of stiffness/induration. The SkinFibroMeter data are presented for the 1-week and 2-week time points for the 6 patients for both the treated and untreated sides. The readings at 4 weeks and beyond leveled out and are not presented.

Investigator Assessment

To assess induration, the investigators were blinded to the treatments. The level of induration was assessed using a graded scale as follows: 0 is none; 1 is barely perceptible; 2 is slight; 3 is moderate; and 4 is severe. All 6 patients were assessed at the follow-up visits at every week initially then at 2 weeks and then every 3 weeks until 10 weeks post procedure/surgery. The data presented are from the 2-week and 4-week follow ups, which correspond to the biopsy time points.

Statistical Analysis

For the gene expression data, paired t-tests (N=3, p<0.05) were performed using TIBCO Spotfire software (Palo Alto, Calif.). The statistical comparison generated delta CT (ddCT) values (the mean dCT of the treated group — the mean dCT of the control group). The statistical software converted the ddCT values into log and linear RQ values for export [RQ=2-ddCT]. The linear RQ values were converted to linear fold-change values to simplify data interpretation. The linear fold-change data was calculated from the exported linear RQ values using Microsoft Excel as follows: for the RQ values>1.0, the linear fold-change value=RQ value, and for the RQ values<1.0, the linear fold-change value=−1/RQ value. The percent change was also provided and calculated as the RQ value minus 1. For the clinical assessment, Student's t-tests were performed using Microsoft Excel. P<0.05 was considered significant.

Results

Assessing the Overall Gene Expression Changes

The biopsies used for gene expression were all from female patients with a mean age of 36 (range 33-38). The gene expression changes for the 2-week and 4-week biopsies were compared to the pre-treatment biopsies for the treated and the untreated samples separately. The results are presented in Table 3. The genes included in the table have a change in gene expression ≥1.5-fold in at least one comparison. Based on these results, the gene expression results were analyzed using the different groups as described below.

TABLE 3
Gene Expression Changes Compared to the Pre-treatment Biopsies

| | |
|---|---|
| 2W vs Pre | 4W vs Pre |

| Gene | UT | T | UT | T |
|---|---|---|---|---|
| AGTR1 | n.s. | 2.09 | n.s. | n.s. |
| BDKRB2 | n.s. | n.s. | n.s. | −1.54 |
| BMP7 | −1.61 | n.s. | n.s. | n.s. |
| BMP8A | n.s. | −1.50 | −1.04 | n.s. |
| C1R | n.s. | 2.50 | n.s. | 1.39 |
| C1S | 3.22 | n.s. | n.s. | n.s. |
| C3 | n.s. | 1.73 | n.s. | n.s. |
| CCL17 | 2.00 | n.s. | n.s. | 3.30 |
| CCL19 | n.s. | n.s. | n.s. | 3.52 |
| CCL20 | n.s. | 3.85 | n.s. | 3.12 |
| CCL22 | n.s. | n.s. | n.s. | 2.19 |
| CCL27 | −2.33 | −2.86 | n.s. | n.s. |
| CCRL1 | 2.42 | n.s. | n.s. | n.s. |
| CCRL2 | n.s. | 1.88 | n.s. | 1.37 |
| CD40LG | n.s. | n.s. | n.s. | 1.70 |
| CD55 | n.s. | n.s. | n.s. | −1.55 |
| CFD | 1.88 | n.s. | n.s. | n.s. |
| CHRNA7 | 2.83 | 1.41 | 1.81 | n.s. |
| CLCF1 | n.s. | n.s. | n.s. | −1.63 |
| CLU | n.s. | 1.55 | n.s. | n.s. |
| CRLF1 | −1.91 | n.s. | −1.91 | −2.05 |
| CYBB | n.s. | 1.64 | n.s. | n.s. |
| EBI3 | n.s. | n.s. | n.s. | 2.62 |
| FIGF | n.s. | 3.19 | n.s. | n.s. |
| FLT3LG | n.s. | n.s. | n.s. | 1.86 |
| GDF15 | n.s. | n.s. | n.s. | 2.47 |
| HGF | 2.48 | n.s. | n.s. | n.s. |
| IL11 | n.s. | n.s. | 2.06 | n.s. |
| IL1F9 | n.s. | n.s. | −1.66 | −1.79 |
| IL21R | n.s. | 1.57 | n.s. | 1.57 |
| IL27RA | n.s. | n.s. | n.s. | 1.52 |
| IL32 | n.s. | n.s. | n.s. | 1.70 |
| IL33 | 2.53 | n.s. | n.s. | n.s. |
| IL3RA | 1.56 | n.s. | n.s. | n.s. |
| IL6 | 1.94 | 3.34 | n.s. | n.s. |
| IL9R | n.s. | −1.61 | n.s. | n.s. |
| ITGB2 | n.s. | 2.84 | n.s. | n.s. |
| KLRG1 | n.s. | 2.18 | n.s. | n.s. |
| LEFTY1 | −1.87 | n.s. | n.s. | n.s. |
| LEPR | n.s. | −2.13 | n.s. | n.s. |
| LTBP4 | n.s. | n.s. | n.s. | −1.52 |
| LTC4S | n.s. | 1.55 | n.s. | n.s. |
| LY86 | n.s. | 2.03 | n.s. | 1.66 |
| MMP25 | n.s. | n.s. | n.s. | 1.52 |
| NFAM1 | n.s. | 1.80 | n.s. | n.s. |
| NFATC4 | n.s. | 1.95 | n.s. | n.s. |
| NLRP12 | n.s. | n.s. | n.s. | 2.29 |
| NLRP3 | n.s. | 3.15 | n.s. | n.s. |
| PLA2G4C | 2.19 | 1.73 | n.s. | n.s. |
| PLCB2 | n.s. | 3.30 | n.s. | 2.21 |
| SCUBE1 | n.s. | n.s. | n.s. | 2.57 |
| SELE | n.s. | n.s. | −2.06 | n.s. |
| SERPINA1 | n.s. | 2.96 | n.s. | n.s. |
| SIGIRR | 1.64 | n.s. | n.s. | n.s. |
| SIGLEC1 | n.s. | 2.39 | n.s. | n.s. |
| SLPI | −1.51 | n.s. | n.s. | n.s. |
| SPN | n.s. | n.s. | n.s. | 1.54 |
| TACR1 | 1.78 | n.s. | n.s. | n.s. |
| TGM2 | n.s. | n.s. | n.s. | 4.26 |
| TNFRSF4 | 2.09 | 1.94 | n.s. | n.s. |
| TNFSF13B | n.s. | 2.33 | n.s. | n.s. |
| TNFSF15 | 1.73 | 2.06 | n.s. | n.s. |
| TPST1 | 2.06 | 1.88 | n.s. | n.s. |

Comparison of the Biopsies Collected at 2 Weeks from the Untreated and Treated Groups

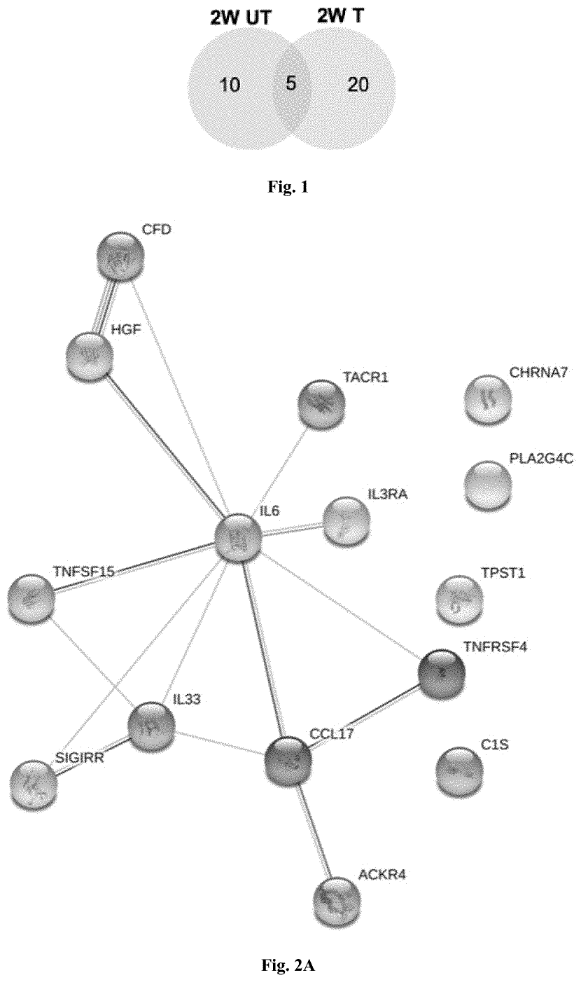

The gene expression analysis of the biopsies collected at 2 weeks (2W) from untreated (UT) and treated (T) groups revealed that 15 genes were significantly upregulated and 5 were significantly downregulated in the untreated group compared to the pre-treatment group. On the other hand, 25 genes were significantly upregulated and 3 were significantly downregulated in the treated group compared to the pre-treatment group. Among the significantly upregulated genes in each group, 5 genes were shared between the untreated and treated groups (FIG. 1). A closer examination of the fold change differences between the 5 genes for the treated and untreated group revealed that the biggest fold change difference between the groups was for the gene interleukin (IL)-6. The difference was 1.94-fold for the untreated, and it was 3.34-fold for the treated group (Table 4).

TABLE 4
Common Genes Between the 2W Untreated and Treated
Groups. All of the values are the fold change
relative to the pre-treatment biopsy samples.

| Gene | 2W UT | 2W T |
|---|---|---|
| IL6 | 1.94 | 3.34 |
| PLA2G4C | 2.19 | 1.73 |
| TNFRSF4 | 2.09 | 1.94 |
| TNFSF15 | 1.73 | 2.06 |
| TPST1 | 2.06 | 1.88 |

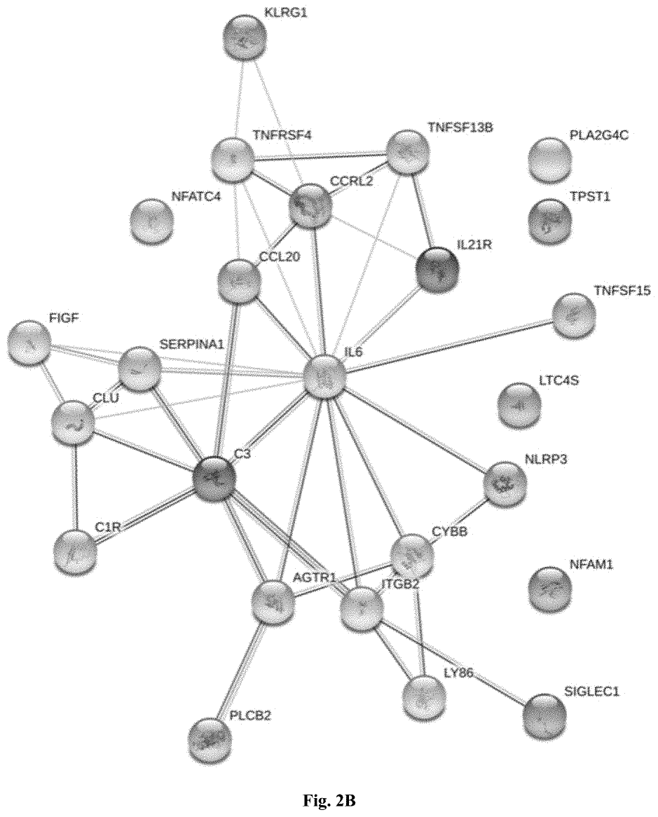

Next, the upregulated genes from the 2 groups were assessed using the String database (https://string-db.org/). The upregulated genes in the untreated and treated groups from the 2-week biopsy samples both centered around IL-6 (FIGS. 2A-2B). Gene Ontology (GO) analysis revealed that each group had a discrete enrichment of GO terms. However, because the 2 groups only shared 5 upregulated genes in common, the details of the inflammatory responses in the 2 groups are unique. For example, for the untreated group, there were 124 biological process GO terms that were significantly enriched, while there were 181 in the treated group. Among these terms, there were 63 in common between the 2 groups. With regard to the unique biological process terms, the top 10 are listed in Table 5. These terms revealed that the untreated group showed a strong pro-inflammatory response, which is expected as a reaction to the procedure. However, for the treated group, this strong inflammatory reaction was altered. No longer was it solely a pro-inflammatory response, the GO terms revealed the activation of the adaptive immune system as well as the effector response, indicating the activation of clearance.

With respect to molecular function, there were 6 significantly enriched GO terms for the untreated group and 15 for the treated group. Among the 6 terms found in the untreated group only 1 (cytokine receptor activity) was unique compared to the treated group. Thus, there were 10 unique terms for the treated group that were indicative of immune modulation (Table 5). For the cellular component GO terms, the untreated group showed no enrichment for these terms, whereas for the treated group there were 19 enriched terms (Table 5). Among these terms, there was strong enrichment for terms related to the plasma membrane, the extracellular space and secretory vessels. Overall, the GO analysis revealed that the untreated groups displays an immune response that was typical after a procedure, while the treatment appeared to have altered the immune response in manner that was promoting clearance and healing.

TABLE 5
Gene Ontology Results from the 2-Week Untreated vs. Treated Comparison

| | |
|---|---|
| 2W UT | 2W T |

Biological Process

| | | | |
|---|---|---|---|
| GO: 0071345 | cellular response to cytokine | GO: 0032940 | secretion by cell |
| | stimulus | | |
| GO: 0001818 | negative regulation of cytokine | GO: 0002443 | leukocyte mediated |
| | production | | immunity |
| GO: 0032675 | regulation of interleukin-6 | GO: 0051716 | cellular response to stimulus |
| | production | | |
| GO: 0045079 | negative regulation of chemokine | GO: 0045087 | innate immune response |
| | biosynthetic process | | |
| GO: 0002526 | acute inflammatory response | GO: 0032103 | positive regulation of |
| | | | response to external stimulus |
| GO: 0032642 | regulation of chemokine production | GO: 0002699 | positive regulation of |
| | | | immune effector process |
| GO: 0045778 | positive regulation of ossification | GO: 0002824 | positive regulation of |
| | | | adaptive immune response |
| GO: 0001976 | neurological system process | GO: 0070887 | cellular response to chemical |
| | involved in regulation of systemic | | stimulus |
| | arterial blood pressure | | |
| GO: 0002830 | positive regulation of type 2 immune | GO: 0002705 | positive regulation of |
| | response | | leukocyte mediated |
| | | | immunity |
| GO: 0033135 | regulation of peptidyl-serine | GO: 0006958 | complement activation, |
| | phosphorylation | | classical pathway |

Molecular Function

| | | | |
|---|---|---|---|
| GO: 0004896 | cytokine receptor activity | GO: 0005102 | signaling receptor binding |
| | | GO: 0001664 | G protein-coupled receptor |
| | | | binding |
| — | | GO: 0005164 | tumor necrosis factor |
| | | | receptor binding |
| | | GO: 0048020 | CCR chemokine receptor |
| | | | binding |
| | | GO: 0005515 | protein binding |
| | | GO: 0038023 | signaling receptor activity |
| — | | GO: 0001540 | amyloid-beta binding |
| | | GO: 0005488 | binding |
| | | GO: 0046982 | protein heterodimerization |
| | | | activity |
| | | GO: 0046983 | protein dimerization activity |

Cellular Component

| | | | |
|---|---|---|---|
| | None | GO: 0005615 | extracellular space |
| | | GO: 0005576 | extracellular region |
| | | GO: 0030141 | secretory granule |
| — | | GO: 0031093 | platelet alpha granule lumen |
| | | GO: 0016020 | membrane |
| | | GO: 0071944 | cell periphery |
| | | GO: 0034774 | secretory granule lumen |
| — | | GO: 0005783 | endoplasmic reticulum |
| | | GO: 0005886 | plasma membrane |
| | | GO: 0016021 | integral component of |
| | | | membrane |
| | | GO: 0044433 | cytoplasmic vesicle part |
| — | | GO: 0005887 | integral component of |
| | | | plasma membrane |
| | | GO: 0012505 | endomembrane system |
| | | GO: 0070821 | tertiary granule membrane |
| | | GO: 0044432 | endoplasmic reticulum part |
| — | | GO: 0044425 | membrane part |
| | | GO: 0035579 | specific granule membrane |
| | | GO: 0005788 | endoplasmic reticulum |
| | | | lumen |
| | | GO: 0030667 | secretory granule membrane |

Finally, the Reactome Pathways (https://reactome.org/) were examined that were enriched in the groups. There were 10 and 19 pathways enriched for the untreated and treat groups, respectively. Among these pathways, there were 6 in common. The 4 unique pathways in the untreated group were related to mitogen and interleukin stimulation as well as PI3K/Akt activation (Table 6). Together these pathways are considered pro-inflammatory regulators. In contrast, the unique pathways enriched in the treated group triggered anti-inflammatory, immunomodulatory and clearance responses. Taken together, the biopsies collected at 2 weeks from the treated and untreated sides showed strikingly different patterns of gene expression. The untreated side revealed typical inflammatory signaling that would be expected in response to the given treatment. In contrast, the treated side revealed that the inflammatory response was more pro-clearance and preparation for healing.

TABLE 6
Reactome Pathway Results from the 2-Week Untreated vs. Treated Comparison

| 2W UT | | 2W T | |
|---|---|---|---|
| | Reactome Pathway | | Reactome Pathway |
| HSA-449147 | Signaling by Interleukins | HSA-168249 | Innate Immune System |
| HSA- | MAPK1/MAPK3 signaling | HSA-198933 | Immunoregulatory |
| 5684996 | | | interactions between a |
| | | | Lymphoid and a non- |
| | | | Lymphoid cell |
| HSA- | PI5P, PP2A and IER3 Regulate | HSA-977606 | Regulation of Complement |
| 6811558 | PI3K/AKT Signaling | | cascade |
| HSA-446652 | Interleukin-1 family signaling | HSA-375276 | Peptide ligand-binding |
| | | | receptors |
| | | HSA-6798695 | Neutrophil degranulation |
| | | HSA-8957275 | Post-translational protein |
| | | | phosphorylation |
| | | HSA-381426 | Regulation of Insulin-like |
| | | | Growth Factor (IGF) |
| | | | transport and uptake by |
| | | | Insulin-like Growth Factor |
| | | | Binding Proteins (IGFBPs) |
| | | HSA-6783783 | Interleukin-10 signaling |
| | | HSA-380108 | Chemokine receptors bind |
| | | | chemokines |
| | | HSA-1280218 | Adaptive Immune System |
| | | HSA-449147 | Signaling by Interleukins |
| | | HSA-109582 | Hemostasis |
| | | HSA-194138 | Signaling by VEGF |

Comparison of the Biopsies Collected at 2 Weeks and 4 Weeks from the Treated Group

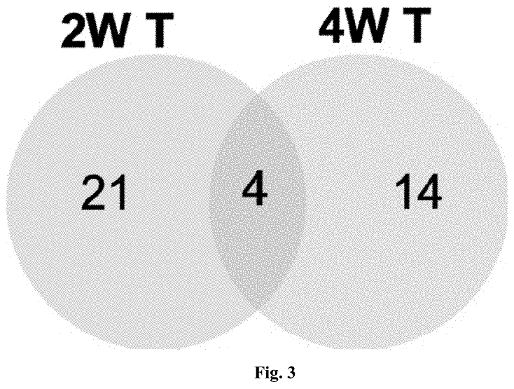

The gene expression data from the biopsy samples collect from the 4-week untreated (UT) group revealed that only 2 genes (CHRNA7 and IL-11) were differentially upregulated in comparison to the pre-treatment sample. These results indicated that by 4 weeks the untreated group had essentially returned to baseline. However, for the 4-week treated group there were 18 significantly upregulated genes compared to the pre-treatment group. Given this observation, the next comparison was between the 2-week and 4-week treated group, with the goal of understanding more about what the treatment was doing to further the activation of the healing patterns that were displayed at the 2 weeks. Among the 25 and 18 significantly upregulated genes in the 2 week and 4 week treated group (Table 3), respectively, there were only 4 in common, including CCL20, IL21R, LY86 and PCLB2 (FIG. 3). All of these showed a pattern of decreased expression between 2 week and 4 weeks, with the exception of IL21R which stayed the same (Table 3).

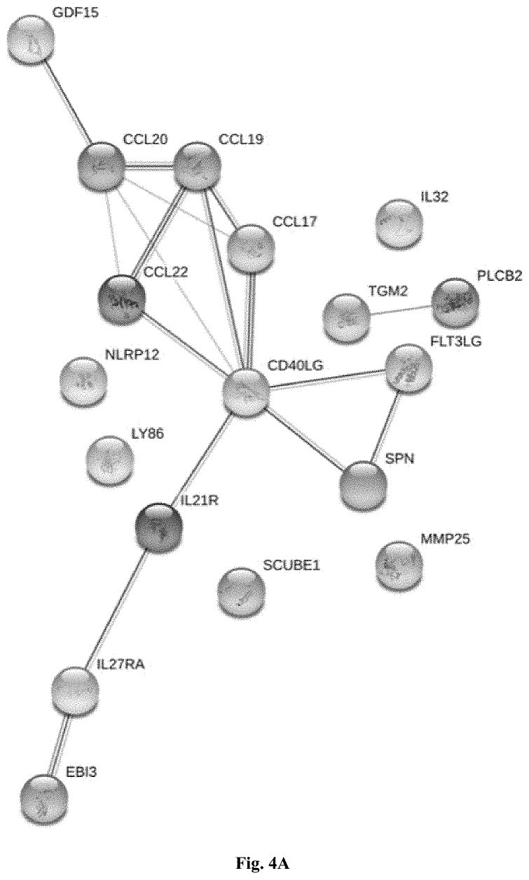

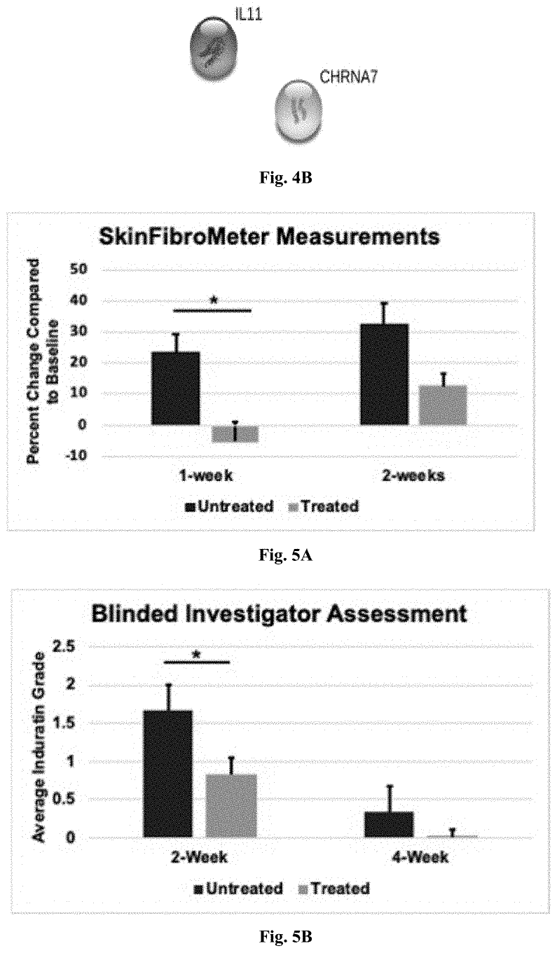

The String Database analysis of the 4-week treated biopsy samples revealed that the protein-protein interactions centered around CD40LG (FIGS. 4A-4B). Furthermore, there were 181 and 115 GO terms for biological process that were significantly enriched for the 2-week and 4-week treated groups, respectively. Among these, there were 57 terms in common, leaving a high level of uniqueness between the groups. With regard to the unique biological process terms, the top 10 from each group are provided in Table 7. As mentioned above, the 2-week treated group showed signs of immunomodulatory action based on the unique biological process terms. This was also true for the 4-week treated group, but in addition, there was an enrichment for terms related to rebuilding and remodeling after injury. For molecular function, the 2-week treated group had 15 enriched terms and there were 7 for the 4-week treated group. Among these terms, 5 were in common, revealing that the molecular functions were similar and were decreasing as time when on with the treatment. For the cellular component, the 2-week treated group had 19 and the 4-week treated group had 4 enriched terms. There was only one term in common, but again the decrease in the number of terms over time went along with the decrease in molecular function terms, indicating more stability in the tissue.

TABLE 7
Gene Ontology Results from the 2-Week Treated vs. the 4-Week Treated Comparison

| 2W T | | 4W T | |
|---|---|---|---|
| Identification | Biological process | Identification | Biological process |
| Go: 0006950 | Response to stress | Go: 0071345 | Cellular response to cytokine stimulus |
| Go: 0002673 | Regulation of acute | Go: 0048247 | Lymphocyte chemotaxis |
| | inflammatory response | | |
| Go: 0032940 | Secretion by cell | Go: 0048583 | Regulation of response to stimulus |
| Go: 0032101 | Regulation of response to | Go: 0050900 | Leukocyte migration |
| | external stimulus | | |
| Go: 0050727 | Regulation of | Go: 0050670 | Regulation of lymphocyte proliferation |
| | inflammatory response | | |
| Go: 0050778 | Positive regulation of | Go: 1902533 | Positive regulation of intracellular signal |
| | immune response | | transduction |
| Go: 0002443 | Leukocyte mediated | Go: 0031640 | Killing of cells of other organism |
| | immunity | | |
| Go: 0051716 | Cellular response to | Go: 0043408 | Regulation of MAPK cascade |
| | stimulus | | |
| Go: 0002252 | Immune effector process | Go: 0061844 | Antimicrobial humoral immune |
| | | | response mediated by antimicrobial |
| | | | peptide |
| Go: 0045765 | Regulation of angiogenesis | Go: 0070372 | Regulation of erk1 and erk2 cascade |
| | Molecular function | | Molecular function |
| Go: 0005102 | Signaling receptor binding | Go: 0008009 | Chemokine activity |
| Go: 0048018 | Receptor ligand activity | Go: 0004896 | Cytokine receptor activity |
| Go: 0001664 | G protein-coupled receptor | | |
| | binding | | |
| Go: 0005164 | Tumor necrosis factor | | |
| | receptor binding | | |
| Go: 0038023 | Signaling receptor activity | | |
| Go: 0001540 | Amyloid-beta binding | | |
| Go: 0005488 | Binding | | |
| Go: 0004888 | Transmembrane signaling | | |
| | receptor activity | | |
| Go: 0046982 | Protein heterodimerization | | |
| | activity | | |
| Go: 0046983 | Protein dimerization | | |
| | activity | | |
| | Cellular component | | Cellular component |
| Go: 0005576 | Extracellular region | Go: 0044421 | Extracellular region part |
| Go: 0030141 | Secretory granule | Go: 0009897 | External side of plasma membrane |
| Go: 0031093 | Platelet alpha granule | Go: 0009986 | Cell surface |
| | lumen | | |
| Go: 0016020 | Membrane | | |
| Go: 0071944 | Cell periphery | | |
| Go: 0034774 | Secretory granule lumen | | |
| Go: 0005783 | Endoplasmic reticulum | | |
| Go: 0005886 | Plasma membrane | | |
| Go: 0016021 | Integral component of | | |
| | membrane | | |
| Go: 0044433 | Cytoplasmic vesicle part | | |
| Go: 0005887 | Integral component of | | |
| | plasma membrane | | |
| Go: 0012505 | Endomembrane system | | |
| Go: 0070821 | Tertiary granule membrane | | |
| Go: 0044432 | Endoplasmic reticulum | | |
| | part | | |
| Go: 0044425 | Membrane part | | |
| Go: 0035579 | Specific granule | | |
| | membrane | | |
| Go: 0005788 | Endoplasmic reticulum | | |
| | lumen | | |
| Go: 0030667 | Secretory granule | | |
| | membrane | | |

Finally, the Reactome Pathways were examined that were enriched in the 2 groups. There were 19 and 9 pathways enriched in the 2-week and 4-week treatment groups, respectively, and 5 of there were shared between the groups (Table 8). The unique pathways in the 2-week group showed evidence of the activation of anti-inflammatory, immunomodulatory and clearance signaling. In contrast, the 4 unique pathways enriched in the 4-week group were related to not only anti-inflammatory signaling but also to macrophage regulation and extracellular matrix (ECM) remodeling. Together, these data provide evidence that the treatment initially activated the immune system to stimulate clearance and protect the tissue from harm. As time went on, by 4 weeks, the treatment abolished the pro-inflammatory signature and stabilized an anti-inflammatory environment for the promotion of new ECM and further healing.

TABLE 8
Reactome Pathway Results from the 2-Week Treated vs. the 4-Week Treated Comparison

| 2W T | | 4W T | |
|---|---|---|---|
| | Reactome Pathways | | Reactome Pathways |
| HSA-168249 | Innate Immune System | HSA-8984722 | Interleukin-35 |
| | | | Signaling |
| HSA-5669034 | TNFs bind their physiological receptors | HSA-9020956 | Interleukin-27 |
| | | | signaling |
| HSA-198933 | Immunoregulatory interactions between | HSA-418594 | G alpha (i) signaling |
| | a Lymphoid and a non-Lymphoid cell | | events |
| HSA-977606 | Regulation of Complement cascade | HSA-1474228 | Degradation of the |
| | | | extracellular matrix |
| HSA-375276 | Peptide ligand-binding receptors | | |
| HSA-6798695 | Neutrophil degranulation | | |
| HSA-166663 | Initial triggering of complement | | |
| HSA-8957275 | Post-translational protein | | |
| | phosphorylation | | |
| HSA-114608 | Platelet degranulation | | |
| HSA-381426 | Regulation of Insulin-like Growth Factor | | |
| | (IGF) transport and uptake by Insulin- | | |
| | like Growth Factor Binding Proteins | | |
| | (IGFBPs) | | |
| HSA-1280218 | Adaptive Immune System | | |
| HSA-109582 | Hemostasis | | |
| HSA-194138 | Signaling by VEGF | | |
| HSA-6785807 | Interleukin-4 and Interleukin-13 | | |
| | signaling | | |

Induration Assessment

To assess induration, first, the SkinFibroMeter readings at 1-week and 2-weeks after the procedure for the treated and untreated sides were compared to the baseline (before the procedure) reading. Compared to the baseline reading, the 1-week untreated group showed a 23% increase, which was a sign of more induration; whereas the 1-week treated group decreased by 5% compared to the baseline, indicating a lower level of induration. These differences were statistically significant (p<0.05) (FIG. 5A). Two weeks after the procedure, the untreated group showed a 32% increase, while the treated group increased by only 12%. These differences were not statistically significant. Taken together, the treated group showed less induration compared to the untreated group.

To further evaluate induration, blinded investigator assessments were used. The data from the 2 and the 4 weeks groups were compared between the treatment groups. At 2 weeks, there was a statistically significant lower induration grade for the treated group. By 4 weeks, the grade was lower for both groups, but was approximately 0 for the treated group (FIG. 5B). This time point was not statistically significant. These data support that the treatment results in a lower level of induration after the procedure.

Conclusion

For subjects undergoing body procedures, a topical treatment of regenerating skin nectar (RSN) and regenerating body complex product (RBP) induces an accelerated healing response that involves the clearance of ‘waste’ products and the induction of anti-inflammatory genes. Furthermore, this topical treatment stimulates ECM remodeling, which ultimately improves the long-term results of the healing process, leading to less induration.

### Example 3

### Ultrasound Imaging and Investigator Assessments Following Liposuction

As in Example 2, a split-body study was performed in order to compare post procedural recovery, histological and gene expression changes of medial thigh liposuction in participants using RSN and RBP on one leg in comparison with a bland moisturizer on the other.

Material and Methods

Study Population

Six female participants, ages 18-58, mean age were enrolled in this MB approved (IntegReview, Austin, Tex.), split leg, randomized, double-blind clinical study. Participants eligible to enroll were consented and assigned a blinded kit that contained RSN and Cetaphil® Lotion (Galderma, Fort Worth, Tex.)-bland moisturizer. In each kit there were 2 blinded bottles exactly alike that were labeled right or left leg. At visit one the participant was instructed to use the assigned product to the medial thigh twice daily, for approximately 2-3 weeks, prior to visit 2-liposuction procedure. Procedures at visit 1 consisted of ultrasound scans, photography, SkinFibrometer measurements and biopsies, for participants that elected. At visit 2, participants had ultrasound scans, photography and SkinFibrometer measurements prior to the liposuction procedure. After the procedure they were given additional blinded products (RSN, RBP and two bland moisturizer bottles). Participants were instructed to continue to apply the two topicals given at visit 1 along with the additional products to each designated leg twice daily for the remainder of the 10-week follow-up. Post procedure visits included weeks 1, 2, 4, 7 and 10. At each follow-up visit, procedures consisted of photography, ultrasound scans, SkinFibrometer measurements, blinded investigator assessments and participant assessments.

Liposuction Procedure

The liposuction ports were identical in each case. Two incisions on each leg. The superior incision along the bikini line in the upper medial groin and the second along the medial aspect of the thigh 10 cm superior to the medial condyle of the knee. The liposuction volume was consistent at 250 cc per medial thigh with similar suction techniques. All participants received compression garments and were instructed to remove twice daily to apply topicals to the designated leg, allowing for the products to absorb and then re-apply garment.

SkinFibrometer

The SkinFibrometer (Delfin Technologies, USA, Miami, Fla.) consists of a 1.25 mm length indenter and two force sensors. The device is briefly pressed against the skin and the contact force is registered. The indenter imposes a constant deformation when the instrument is in full contact with the skin. The skin and the underlying upper subcutis resist the deformation and the induration value in Newtons (N) is determined. Measurements were taken at every visit and pre-procedure at Visit 2. The SkinFibrometer was placed at 8, 10 and 12 cm from the top of the medial thigh on each leg, remote from the biopsy sites and three measurements were recorded at each location

Biopsies

Of the 6 participants enrolled in the study, 5 consented to having punch biopsies, ages 18-43, mean age 34. A 3mm punch biopsy was performed at 3 visits on the right and left leg (visit 1-pre topical application, 2- and 4-weeks post liposuction). All punch biopsies were completed in the same location, with sites remote from the liposuction ports. The first biopsy was 8 cm inferior to the adductor insertion, the second biopsy was 10 cm and the third was 12 cm inferior, along a line drawn from the insertion of the adductor brevis on the inferior ramus of the pubis to the medial condyle of the femur at the knee. The first three participant biopsy sets were sent to Genemarkers (Kalamazoo, Mich.) for gene expression analysis. Paired t-tests (N=3, p<0.05) were performed using TIBCO Spotfire software for the gene expression analyses. The other two participants biopsies were sent to Laboratory and Pathology Diagnostics (Naperville, Ill.) for dermatopathology analyses.

Ultrasound Imaging

Using the Episcan 1-200 with a 20 MHz probe (Longport, Inc., USA, Chadds Ford, Pa.), ultrasound images were taken on each leg at three measured locations (8, 10 and 12 cm from the top of the medial thigh), remote from the biopsy sites. Images were captured at 5, 10, 15 and 20 mm depths at baseline, pre-topical application, and pre-procedure at visit 2 and at every follow-up visit. To measure the transition/difference from the dermal to subcutaneous tissue, the difference between the dermal and subcutaneous intensity analysis was calculated. RDIS is relative dispersion, or a measure of relative variation. A higher value denotes more echogenic or a brighter image, which in turn indicates denser tissue. Induration is represented by disruption of the subcutaneous dermal interface with infiltration of fluid from subcutaneous region to dermal segment. In such cases, the compact nature of the dermis is disrupted, resulting in a less dense dermis.

Investigator Assessments

At weeks 1, 2, 4, 7 and 10 post liposuction to the medial thighs, the blinded investigator (PI), assessed each leg for induration, edema, skin discoloration, ecchymosis, subcutaneous banding and pain. Each assessment was made using a 0-4—point scale (0=none, 1=barely perceptible, 2=slight, 3=moderate and 4=severe). The VAS pain scale was also used to assess pain (0-10 scale).

Participant Assessments

At each visit following the liposuction procedure each participant was given a questionnaire to assess each leg on a 0-3-point scale (0=none, 1=mild, 2=moderate and 3=severe). Questions included: Do you have sensitivity to the touch? Do you have swelling in the area? Does the area feel numb? Do you have bruising/discoloration? Do you have pain/discomfort? Do you have redness?

Results

Investigator Assessments

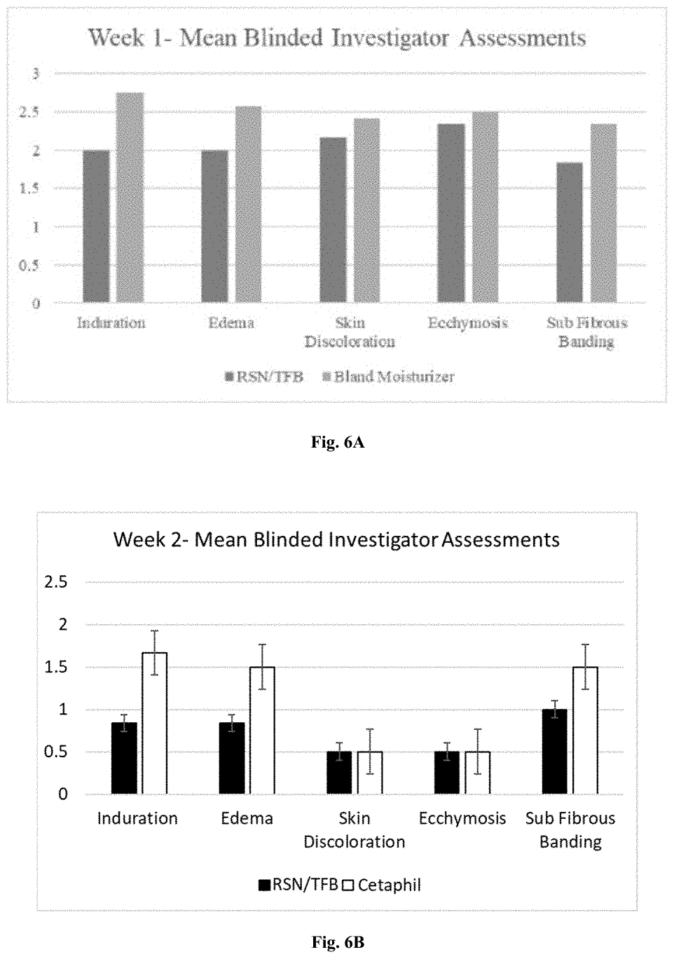

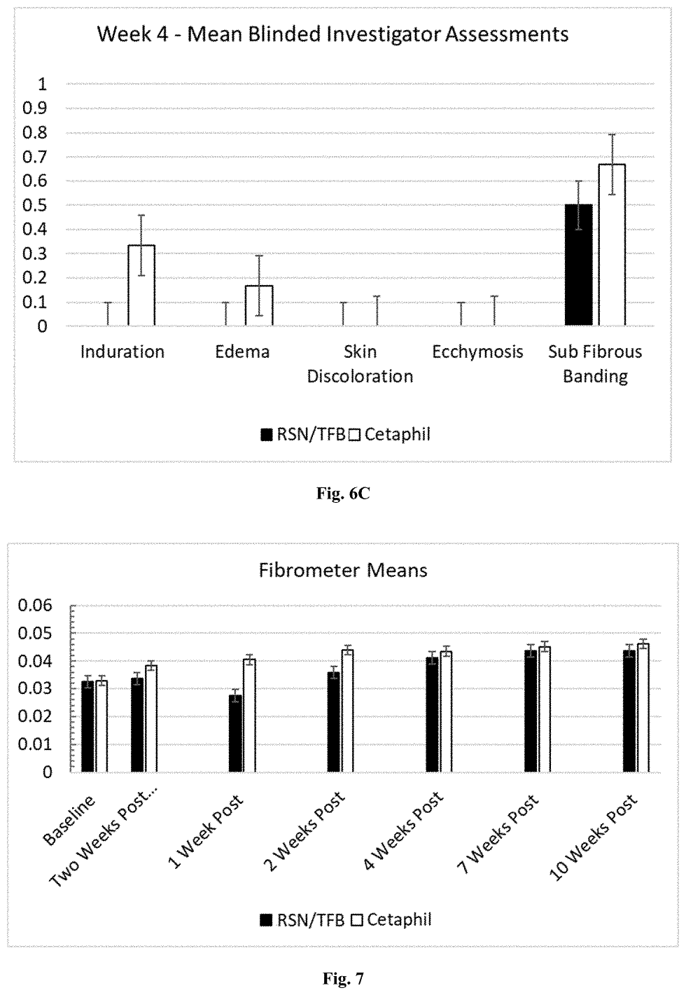

Post-liposuction, the mean blinded investigator assessments of induration, edema and subcutaneous fibrous banding had less severity at weeks 1, 2 and 4 on the leg using the RSN and RBP (topical treatment). At week 1, skin discoloration, ecchymosis and pain on the VAS were less on the side using the topical treatment (FIG. 6A). At week 2, there was a statistically significant difference in induration between each leg, 0.833 for the topical treatment leg and 1.66 on the bland moisturizer leg (p=0.05) (FIG. 6B). Edema was also on trend with 0.833 for the topical treatment leg and 1.5 for the bland moisturizer leg. Subcutaneous banding was 1.0 for the topical treatment side and 1.5 for the bland moisturizer leg. At week 4, only mild subcutaneous banding was present on the topical treatment side (0.5), while on the bland moisturizer side, induration (0.33), edema (0.16) and subcutaneous banding (0.7) were present (FIG. 6C). At week 7, induration persisted on some of the participants leg using bland moisturizer. Edema increased slightly on both sides, which is typically seen clinically post liposuction, due to lifestyle. By week 10, all post procedural healing assessments had resolved on the side using the topical treatments, while induration and subcutaneous banding were still present in some of the participants side using bland moisturizer.

SkinFibrometer Measurements

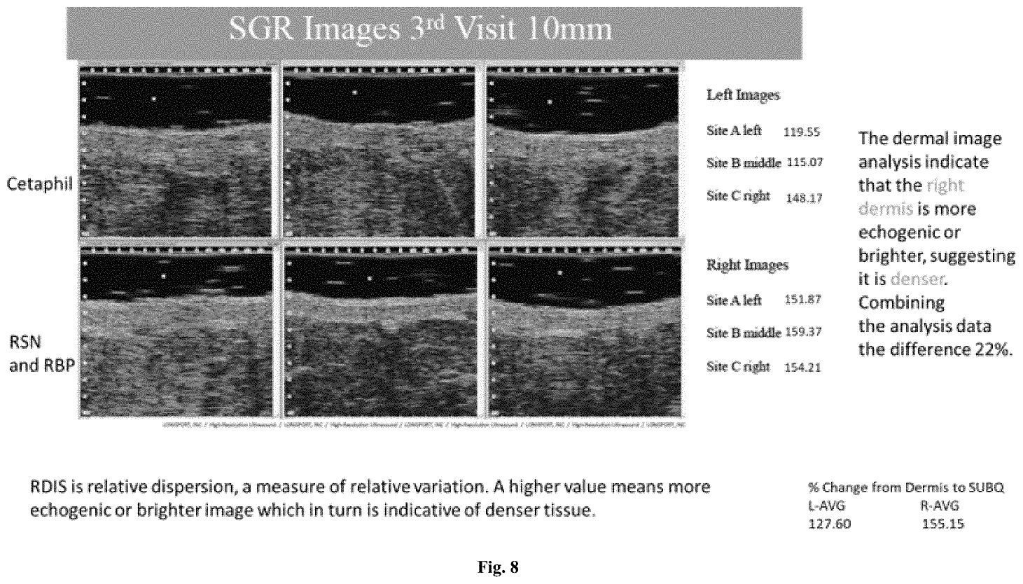

The mean change was calculated for each leg at every visit. Again, week 1 and 2 post procedure showed the greatest difference in results with the side having the experimental topical treatment of RSN and RBP showing the least change from baseline whereas the leg using Cetaphil had a greater increase from baseline indicating an increase in induration (FIG. 7). Although not statistically significant, this trend is maintained throughout the first 4 weeks.

Ultrasound Analysis

Ultrasounds were analyzed by a blinded independent evaluator from Longport, Inc. Clinically the major differences in induration were noted at 2 weeks, therefore ultrasound analysis was focused at this time period. To measure the transition from the dermal to subcutaneous tissue the difference between the dermal and subcutaneous intensity analysis was calculated. RDIS is relative dispersion, a measure of relative variation. A higher value means more echogenic or a brighter image which in turn is indicative of denser tissue. Induration is represented by disruption of the subcutaneous dermal interface with infiltration of fluid from subcutaneous region to dermal segment, thus disrupting the compact nature of the dermis resulting in a less dense dermis.

5 out 6 patients showed increased average density over 3 areas (less fluid infiltration, edema, induration) on the RSN and RBP side over the comparator at the 2-week time point (FIG. 8). Even in the 1 patient that did not show overall advantage on the RSN/RBP side, the ultrasound in fact showed less induration in the middle image on the RSN/RBP side. Put another way, out of the 18 comparison points, the leg using RSN and RBP proved to have less induration in 15/18 images (83.3%). In addition, taking into account the middle area of the analyzed tissue, which is representative of the most consistent area for liposuction, all 6 patients showed improved results on the side using RSN and RBP.

Histological Analysis

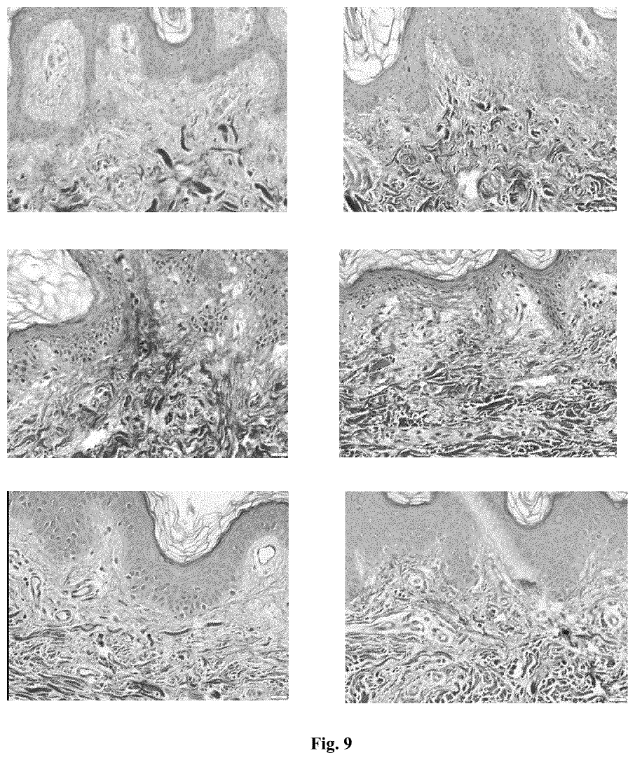

The Herovici stain has been particularly useful to demonstrate new collagen formation by staining newly laid down mucopolysaccharide components in the papillary dermis denoting early collagen regeneration. This serves to establish the regenerative changes in the ECM that would be sought with products initiating anti-aging changes in the skin. Analysis of the biopsies from the dermatopathologist show a marked increase in neocollagenesis (Herovici stain) particularly on the RSN/RBP side (FIG. 9). In addition, increased fibrillin (elastin component was consistently elevated at 4 weeks in both patients on the RSN/RBP side.)

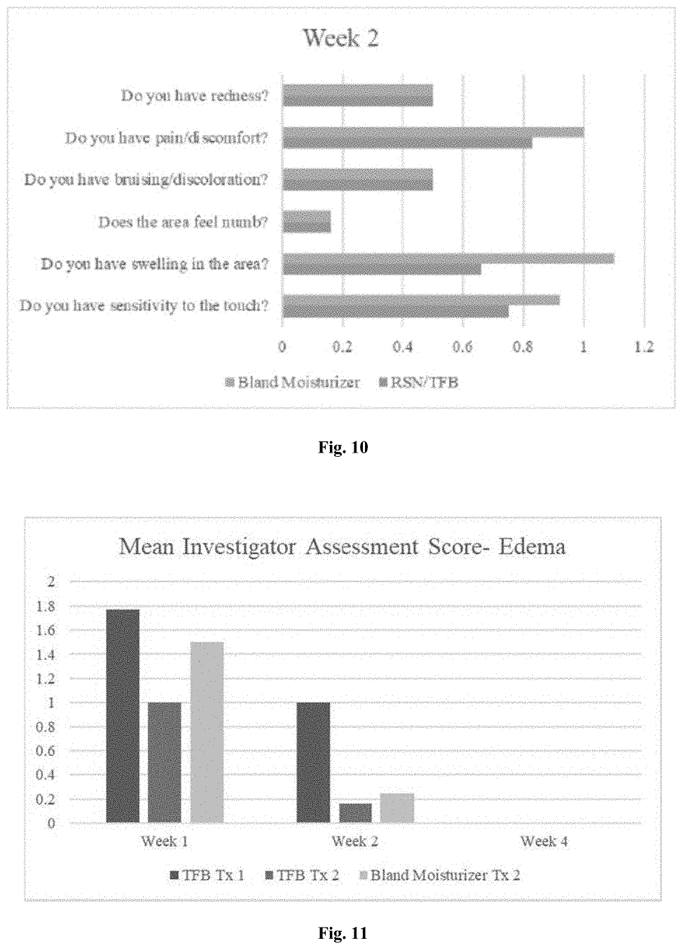

As far as the participant assessments are concerned, in keeping with signs and symptoms being maximal within the first 2 weeks, swelling on the RBP side was assessed as less severe by the majority of participants at week 2, mean average score for swelling on the RBP side 0.66 and 1.1 on the Cetaphil leg. It is also of note, that 5 of the 6 participants selected to use the topical treatments with their next body procedure (FIG. 10).

Conclusion

Using RSN to prepare the skin for surgical procedures combined with RSN and RBP post procedure has been correlated through clinical study outcomes, histological evidence and finally gene analysis demonstrating remodeling of the extracellular matrix, accelerating healing and initiation of anti-inflammatory genes. The investigator assessments, participant assessments and induration measurements align with less severe post procedural events and match up with the genes being expressed within the 4 weeks post procedure period.

### Example 4

### Assessments of Post-Procedural Healing with DCA Injections

This study evaluated post-procedural healing and outcomes of submental deoxycholic acid (DCA) injections in conjunction with RBP post-treatment.

Materials and Methods

Study Population

This single center, randomized, crossover trial evaluated the use of RBP post submental DCA injections. Twelve participants, 3 males and 11 females, ages 25-63 (mean age 42), with clearly visible submental subcutaneous fat, with soft, pliable tissue of sufficient volume for treatment, were included. Those with previous fat reduction procedures or implants in or near the treatment area, scars, excessive laxity, previous surgery in the treatment region and/or any contra-indications to DCA usage, as determined by the physician, were excluded from participating in the study. Women pregnant or lactating or planning on becoming pregnant during the study duration were also excluded. Participants were consented prior to any study procedures and the study was conducted under applicable regulations in accordance with the Declaration of Helsinki and the International Conference on Harmonisation Tripartite Guidelines for Good Clinical Practice.

Administration of DCA Injections

At Visit 1- Baseline (BL), all eligible participants were consented, standardized photography and assessments were completed, and submental DCA injections were performed. The average volume injected was 8 cc. Post procedure, all participants were given RBP in a blinded bottled to use twice daily to the treatment area.

Follow-Up Treatments

Participants returned at weeks 1, 2 and 4 post treatment for follow-up and assessments. At the week 4 follow-up visit, participants returned the RBP and washed out for 30 days prior to treatment two. At treatment two, participants were randomized to receive either RBP or Cetaphil® Lotion (Galderma, Fort Worth, Tex.), in a blinded bottle-identical, to apply twice daily, post DCA injections, and return to the office for follow-up at weeks 1, 2 and 4. Follow-up procedures post treatment two were the same as post treatment one. Average volume injected at treatment two was 7.7 cc.

Investigator Assessments

At weeks 1, 2 and 4 post both DCA treatments and prior to the second DCA treatment, the blinded investigator, (PI), assessed the submental treatment area for induration, edema, erythema, bruising and pain. Each assessment was graded using a (0-4) point scale. The VAS, a (0-10) point scale, was used to score pain. Submental fullness (CR-SFRS)7was graded at every visit using the submental fat rating scale, (0-4) point scale.

SkinFibrometer

These objective measurements were taken at every visit at three locations (right, middle and left) over the submental treatment area to measure induration. The SkinFibrometer (Delfin Technologies, USA, Miami, Fla.) consists of a 1.25 mm length indenter and two force sensors. The device is briefly pressed against the skin and the contact force is registered. The indenter imposes a constant deformation when the instrument is in full contact with the skin. The skin and the underlying upper subcutis resist the deformation and the induration value in Newtons (N) is determined.

Statistical Analysis

These objective measurements were taken at every visit at three locations (right, middle and left) over the submental treatment area to measure induration. The SkinFibrometer consists of a 1.25 mm length indenter and two force sensors. The device is briefly pressed against the skin and the contact force is registered. The indenter imposes a constant deformation when the instrument is in full contact with the skin. The skin and the underlying upper subcutis resist the deformation and the induration value in Newtons (N) is determined.

Results

Treatment 1: One-Sample T-Test

Since all patients were given RBP in the first treatment of deoxycholic acid, a one-sample t-test was used to detect significant changes in the outcomes from baseline to week 4. The mean score of CR-SFRS, a measure for fat reduction, was decreased by −0.8 units from baseline to week 4. This decrease was statistically significant (p=0.0020). These results were anticipated and served as baseline comparators for Treatment 2.

Treatment 2: Investigator Assessments

After treatment 2, less edema and induration on the RBP side were noted by the blinded investigator assessment. In fact, at 1-week post treatment 2, the degree of edema was equivalent to that of 2 weeks post treatment 1.

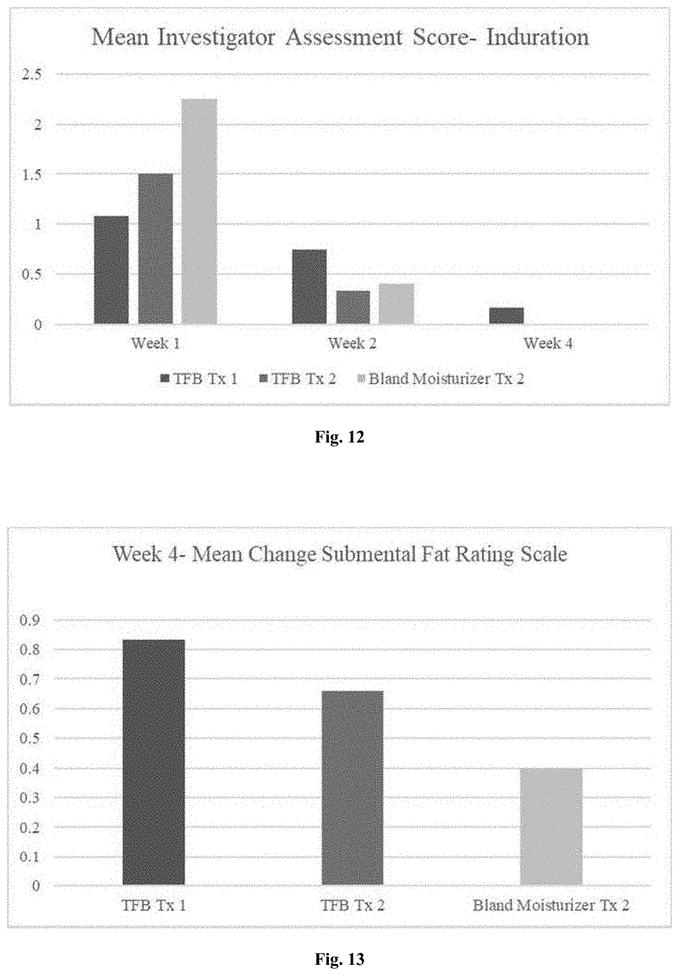

At week 1 post treatment two, participants using RBP had 44% less edema than post treatment one compared to 15% less edema in the participants using the bland moisturizer (FIG. 11). Investigator assessment of induration tended to follow a similar pattern (FIG. 12). It is of note, that due to the second treatment being so close to the first treatment (approximately 60 days), there may have been some residual induration at the post treatment 2. As far as the fat rating scale assessments, CR-SFRS, at 4 weeks, post treatment one, participants had a statistically significant decreased score on the submental fat scale which trended through post treatment two for the participants using RBP (FIG. 13).

Erythema, bruising, and pain were not observed post treatment two in either group and post treatment one there was one incidence of pain and two incidents of mild bruising.

Treatment 2: SkinFibrometer Measurements

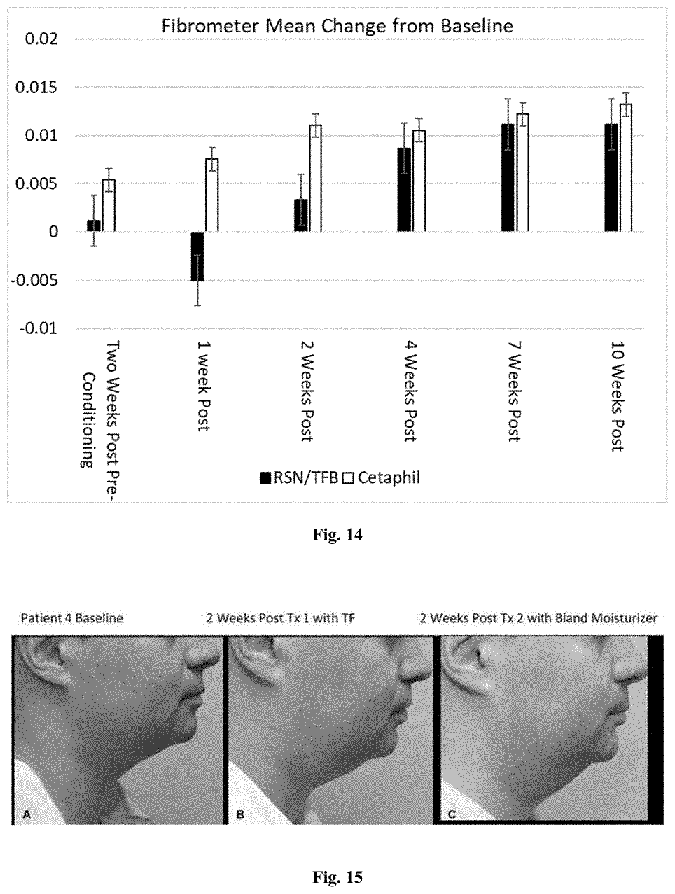

After the second treatment, participants using RBP had statistically significant less induration than participants using bland moisturizer in all three areas measured submentally at week 2 (FIG. 14). The mean scores of SkinFibrometer Right Lateral (RL) at week 2 after treatment 2 were 0.00033 among the six RBP recipients and 0.0068 among the five Bland moisturizer recipients. This difference was statistically significant (p=0.0319). The mean scores of SkinFibrometer Midline (MID) at week 2 after treatment 2 were 0.0031 among the six RBP recipients and 0.014 among the five Bland moisturizer recipients. This difference was statistically significant (p=0.0033). The mean scores of SkinFibrometer Left Lateral (LL) at week 2 after treatment 2 were -0.0045 among the six RBP recipients and 0.0047 among the five Bland moisturizer recipients. This difference was statistically significant (p=0.0182).

Lastly, when comparing treatments 1 and 2 with SkinFibrometer readings at 2 weeks post treatment using midline score averages (area most affected), it was very apparent that the second treatment—which should have less reaction than the first—does so with the RBP group (RBP lsttreatment average 0.006625 vs 0.0031). However, when comparing this score with the bland moisturizer at the same time, a statistically significant difference (RBP 1s^{t} treatment average 0.006625; RBP 2^{nd} treatment 0.0031; bland moisturizer 0.014) was observed (FIG. 14).

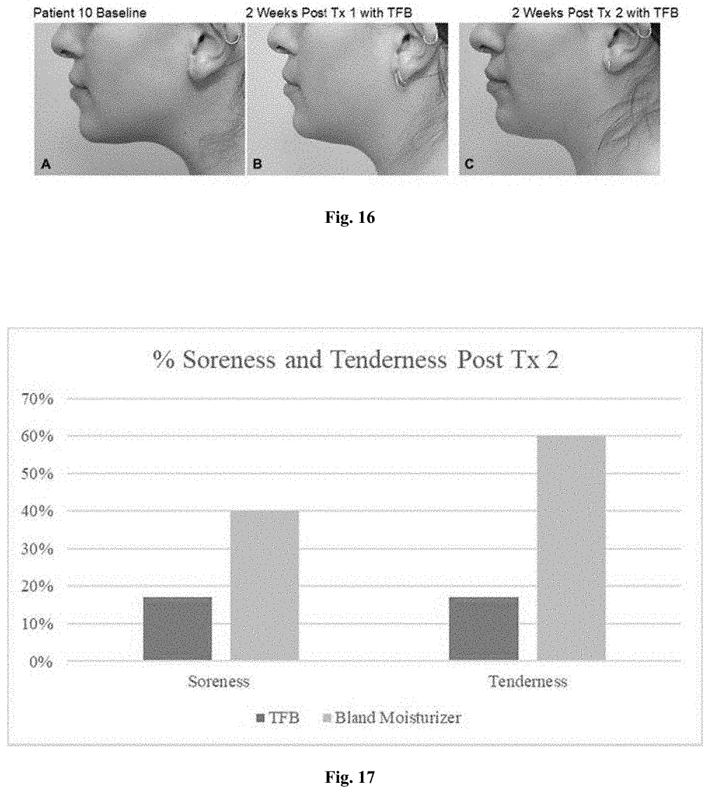

Typical participant photos are shown in FIGS. 15 and 16.

Of the patient reported adverse reactions, post treatment two, the greatest difference was in tenderness and soreness with 17% reported in the RBP compared to the Bland moisturizer group with 60% tenderness and 40% soreness (FIG. 17).

Conclusion

Investigator assessments and objective SkinFibrometer analyses have demonstrated that the use of RBP post DCA injections may reduce induration, edema and discomfort associated with this procedure. Improvement in patient experience and outcome is a welcome adjunct to any procedure, particularly those needing repetitive sessions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for improving wound healing after a skin procedure or surgical procedure in a subject, the method comprising:
(a) applying to a skin region of the subject a first topical composition comprising a tripeptide-1 and hexapeptide-12 before the skin procedure or the surgical procedure; and
(b) applying to the skin region of the subject a second topical composition comprising tripeptide-1, a hexapeptide-12, and a hexapeptide-11,
wherein the first topical composition, the second topical composition, or both, is administered in an amount sufficient to modulate an expression level of one or more inflammatory or regenerative genes.

2. The method of claim 1, wherein step (b) further comprises applying the first topical composition.

3. The method of claim 1, wherein the first topical composition is administered for at least one week before the surgical procedure.

4. The method of claim 1, wherein the second topical composition is administered for at least two weeks after the surgical procedure.

5. The method of claim 1, wherein the first topical composition, the second topical composition, or both is administered one, two, three, four, five, or six times a day.

6. The method of claim 1, wherein the first topical composition is administered at least two times a day for at least two weeks before the skin procedure or the surgical procedure and the second topical composition is administered at least two times a day for at least two weeks after the skin procedure or the surgical procedure.

7. The method of claim 1, wherein the one or more inflammatory or regenerative genes encodes a chemokine receptor, a chemokine-like receptor, a chemokine ligand, an angiotensin receptor, a complement component, a cholinergic receptor, a clusterin, a cytochrome, a fibroblast growth factor, a growth differentiation factor, a hepatocyte growth factor, an interleukin receptor, an interleukin, an integrin, a killer cell like lectin receptor, a leukotriene synthase, a lymphocyte antigen, a matrix metallopeptidase, a nuclear factor of activated T-cell, a Nod-like receptor (NLR), a phospholipase, a serpin, a single Ig IL-1-related receptor, a sialic acid binding Ig like lectin, a signal peptide, a CUB domain and EGF like domain, a sialophorin, a tachykinin receptor, a TNF receptor, a transglutaminase, a tyrosylprotein sulfotransferase, or a fragment or variant thereof.

8. The method of claim 1, wherein the one or more inflammatory or regenerative genes comprises ACKR4, AGTR1, C1R, C1S C3, CCL17, CCL19, CCL20, CCL22, CCR19, CCRL2, CD40LG, CFD, CHRNA7, CLU, CYBB, EB13, FIGF, FLT3LG, GDF15, HGF, IL11, IL21R, IL27RA, IL32, IL33, IL3RA, IL6, ITGB2, KLRG1, LTC4S, LY86, MMP25, NFAM1, NFATC4, NLRP12, NLRP3, PLA2G4C, PLCB2, SCUBE1, SERPINA1, SIGIRR, SIGLEC1, SPN, TACR1, TGM2, TNFRSF4, TNFSF13B, TNFSF15, or TPST1.

9. The method of claim 8, wherein the one or more inflammatory or regenerative genes comprises IL6, C1R, SCUBE1, TGM2, or PLCB2.

10. The method of claim 1, wherein the expression level is increased by at least 1.25-fold as compared to control, wherein the control is a baseline expression level or the control is a skin region that does not receive the first topical composition, the second topical composition, or both.

11. The method of claim 1, wherein the tripeptide-1 is present at 1-10 ppm.

12. The method of claim 1, wherein the hexapeptide-11 is present at 0.001-1 ppm

13. The method of claim 1, wherein the hexapeptide-12 is present at 0.5-10 ppm.

14. The method of claim 1, wherein the second topical composition further comprises tetrapeptide-2, wherein the tetrapeptide-2 is present at 1-10 ppm.

15. The method of claim 1, wherein the first topical composition comprises phosphatidylserine, wherein the phosphatidylserine is present at no more than 5.0 wt. %.

16. The method of claim 1, wherein the first topical composition comprises oleuropein, wherein the oleuropein is present at no more than 0.050 wt. %.

17. The method of claim 1, wherein the second topical composition further comprises phosphatidylserine, wherein the phosphatidylserine is present at no more than 5.0 wt. %.

18. The method of claim 1, wherein the first topical composition, the second topical composition, or both, further comprises ceramide NP, Tremella fuciformis extract, niacinamide, hydrogenated lecithin, C12-16 alcohols, palmitic acid, avocado extract, shea butter, bentonite, phytoene/phytofluene, hydroxymethoxyphenyl decanone, polyholosides, Plantago lanceolata, dill extract, hydrolyzed Candida saitoana extract, Centella asiatica, propanediol, lecithin, Euglena gracilis extract, water, caffeine, Glaucium flavum leaf extract, or combinations thereof.

19. The method of claim 1, wherein:
the skin procedure comprises a laser treatment, a chemical peel, microdermabrasion, microneedling, or radiofrequency microneedling; or
the surgical procedure comprise a panniculectomy, liposuction, a lower body lift, brachioplasty, an inner thigh lift, a buttock augmentation, a circumferential body lift, a breast lift, a breast reduction, a breast augmentation, or a labiaplasty.

20. The method of claim 1, wherein the method accelerates healing, accelerates clearance of products including lipid particles, accelerates resolution of inflammation stimulates extracellular matrix remodeling, reduces induration, reduces fibrous banding, reduces pain or discomfort, or combinations thereof.
